(19) United States
(12) Patent Application Publication
(10) Pub. No.: US 2025/0000913 A1
(43) Pub. Date: Jan. 2, 2025

(54) SALMONELLA ENGINEERED FOR NONTOXIC COLONIZATION OF TUMORS

(71) Applicant: Regents of the University of Minnesota, Minneapolis, MN (US)

(72) Inventors: Daniel Alan Saltzman, Mendota Heights, MN (US); Janet Louise Schottel, Roseville, MN (US); Lance Bischoff Augustin, Hudson, WI (US)

(21) Appl. No.: 18/690,594

(22) PCT Filed: Sep. 9, 2022
(86) PCT No.: PCT/US2022/043112

§ 371 Date: Mar. 8, 2024

Related U.S. Application Data
(60) Provisional application No. 63/242,975, filed on Sep. 10, 2021.

(51) Int. Cl. A61K 35/74; A61K 38/20; A61P 35/00; C12N 1/36; C12N 15/74

(52) U.S. Cl. CPC A61K 35/74; A61K 38/204; A61P 35/00; C12N 1/36; C12N 15/74; C12N 2800/101

(57) ABSTRACT

Provided herein is the ability for intravenous delivery of bacteria without the septic side-effects and for delivery of immune modulating proteins that are systemically toxic to be delivered directly to the tumor micro-environment without any systemic toxicity.

### PRIORITY APPLICATION(S)

This application claims the benefit of priority to U.S. Provisional Patent Application Ser. No. 63/242,975, filed on Sep. 10, 2021, the disclosure of which is incorporated by reference herein in its entirety.

### BACKGROUND OF THE INVENTION

Much of the bacterial anticancer therapy being developed relies on the ability of bacteria to specifically colonize tumors. Initial attempts to translate Salmonella enterica Typhimurium (S. Typhimurium) preclinical results to the clinical setting failed, primarily due to lack of tumor colonization and the significant toxicities from systemically administered Gram-negative bacteria.

### SUMMARY OF THE INVENTION

Provided herein is the reduction of toxicity due to interaction of bacterial surface molecules with receptors on mammalian cells. This is accomplished by editing the bacterial genome to eliminate flagellin, fimbriae, O-antigen and lipopolysaccharide proteins that bind to toll-like receptors on mammalian cells. In addition, expression of an outer membrane protease that inhibits complement activation was increased. Further, toxicity is reduced/eliminated due to systemic administration of immunomodulator proteins. This is accomplished by bacterial strains containing plasmids with genetic cassettes for expression and secretion of proteins into the tumor microenvironment, resulting in an anticancer immune response with reduced systemic toxicity. Thus, provided herein is the ability for intravenous delivery of bacteria without the septic side-effects and for delivery of immune modulating proteins that are systemically toxic to be delivered directly to the tumor micro-environment without any systemic toxicity.

One aspect provides an attenuated Salmonella cell comprising: a) a mutation or deletion in one or more of the Salmonella genes coding for cell surface proteins that induce IL-6 secretion, so as to result in reduced or no expression of said one or more proteins; and optionally b) an increase in expression, as compared to a control cell, of one or more outer membrane proteases that inhibit complement activation and/or a decrease in expression of cell surface lipopolysaccharide protein (LPS). In one aspect, the cell is a S. Typhimurium cell. In one aspect, the one or more Salmonella genes code for flagellin, fimbriae, O-antigen and/or lipopolysaccharide protein (LPS). In another aspect, the one or more Salmonella genes are fliC, fljB, fimH and/or rfaL. In one aspect, the one or more outer membrane proteases is PgtE.

One aspect further comprises a deletion of the enterobacterial common antigen locus (eca). Another aspect further comprises a deletion of the rpoS gene and/or the addition of a viaB locus. Another aspect further comprises a deletion of one or more of fljA, rflP, flgKL and/or motAB genes. In some aspects, the endogenous flhDP promoter of the Salmonella is replaced with a tumor-specific expression promoter. In one embodiment, the tumor-specific expression promoter is FF+20* promoter.

In one aspect the cell comprises one or more exogenous immunomodulator genes which express an exogenous immunomodulator protein. In some aspects, the immunomodulator genes code for/express IL-12, IL-18, IL-15, CXCL9-10, αCTLA-4 single-chain fragment variable (scFv), αPD-L1 scFv, αCTLA-4 single-domain antibody (sdAb), αPD-L1 sdAb and/or αCD47 sdAb protein. In one aspect, the immunomodulator protein is secreted from the cell. In some aspects, the one or more exogenous immunomodulator genes are under the control of a tumor-specific expression promoter. In one aspect, the tumor-specific expression promoter is FF+20*.

One aspect provides for a composition comprising a population of cells described herein or a combination thereof and a pharmaceutically acceptable carrier.

One aspect provides a method treat cancer comprising administering to subject in need thereof an effective amount of a population of the cells as described herein, a combination thereof or the composition described herein so as to treat said cancer.

One aspect provides a method of inhibiting tumor growth/proliferation or reducing the volume/size of a tumor comprising administering to subject in need thereof an effective amount of a population of the cells described herein, a combination thereof or the composition described herein so as to suppress tumor growth or reduce the volume of the tumor.

Another aspect provides a method to treat, reduce formation/number or inhibit spread of metastases comprising administering to subject in need thereof an effective amount of a population of the cells described herein, a combination thereof or the composition described herein, so as to treat, reduce formation/number or inhibit spread of metastases.

In one aspect, the tumor, cancer, or metastases are a lung, liver, kidney, breast, prostate, pancreatic, colon, head and neck, ovarian and/or gastroenterological tumor, cancer or metastases.

In another aspect, the cells or composition is administered systemically. In one aspect, the cells are administered more than once. Another aspect further comprises administering a vascular disrupting agent (VDA) and/or cannabidiol (CBD). In one aspect, the VDA and/or CBD are administered prior to and/or during treatment (after at least one administration of the cells). In aspect the VDA and/or CBD are administered more than once. In one aspect, the VDA is VDA combretastatin A4 phosphate and or VDA CKD-516. In one aspect, anti-interleukin-6 (IL-6) is administered.

One aspect is a method to reduce toxicity of Salmonella comprising a) deleting one or more of the Salmonella genes coding for cell surface proteins that induce IL-6 secretion, so as to result in reduced or no expression of said one or more proteins; and optionally b) increasing expression, as compared to a control cell, of one or more outer membrane proteases that inhibit complement activation and/or decreasing expression of cell surface lipopolysaccharide protein (LPS). In one embodiment, the Salmonella is a S. Typhimurium cell. In one aspect, the one or more Salmonella genes code for flagellin, fimbriae, O-antigen and/or lipopolysaccharide protein (LPS). In another aspect, the one or more Salmonella genes are fliC, fljB, fimH and/or rfaL. In one aspect, the one or more outer membrane proteases is PgtE. One aspect further comprises a deletion of the enterobacterial common antigen locus (eca). Another aspect further comprises a deletion of the rpoS gene and/or the addition of a viaB locus. A further aspect comprises a deletion of one or more of fljA, rflP, flgKL and/or motAB genes. In one aspect, the flhDP promoter is replaced with a tumor-specific expression promoter. In another aspect, the tumor-specific expression promoter is FF+20* promoter.

### BRIEF DESCRIPTION OF THE DRAWINGS

FIG. 1. Anti-IL-6 antibodies reduce toxicity of systemic S. Typhimurium plus a VDA. BALB-neuT female mice bearing 400 mm³ tumors were treated IV with either 5×10⁵ or 1×10⁶ cfu of strain BCT1(pNG). Bacterial cfu numbers indicate total cfu delivered on day 0 with 50% of the bacteria given in two injections three hours apart. All mice received IV CA4P VDA by two injections of 0.4 mg per injection separated by three hours, administered on days −2 and −1. Where indicated, 1 mg anti-IL-6 mAb was administered IP immediately following the first S. Typhimurium injection. Mice were monitored for weight loss and survival after treatment for seven days. The surviving mice/total mice treated in each group is depicted as a fraction below each column. The y-axis values represent the average percent weight loss of the surviving mice in each group. Error bars indicate standard deviation from the mean.

FIG. 2. Construction of strain BCT2. S. Typhimurium strain BCT2 was constructed by introducing the ΔfliC, ΔfljB, ΔfimH, pgtEp(NC_016863.1: g.2506794G>A), and ΔrfaL mutations into strain χ11091 (contains the lipid A mutations (23)) to modify surface molecules to avoid systemic induction of CRS. The genes are listed in the last column, the molecules they code for are in the middle column, and the corresponding immune response is listed in the first column.

FIG. 3. S. Typhimurium BCT2 toxicity. Weight change following bacterial administration to non-tumor burdened BALB/c mice with 1×10⁷ cfu (closed symbols) or 1×10⁶ cfu (open symbols) of strains VNP20009 or BCT2(pPflEPLux) was followed for 7 days post bacterial injection. Error bars (shown in a single direction for clarity) represent standard deviation of measurements from 4 mice.

FIG. 4. S. Typhimurium BCT2 toxicity and tumor colonization. Weight change was followed in tumor-burdened BALB-neuT mice treated with strain BCT2(pFF+20*Lux) and a VDA. The CKD-516 VDA was administered as a single 0.1 mg IP injection on days −4, −3, −2 and −1, prior to IV administration of two 1.5×10⁶ cfu BCT2(pFF+20*Lux) doses separated by three hours on day 0, followed an hour later with a 0.05 mg IP injection of CKD-516. Error bars represent standard deviation of measurements from 3 mice. Insert: Bioluminescence from tumors in the three BALB-neuT mice treated with BCT2(pFF+20*Lux) and the VDA. Four views of each mouse are shown.

FIG. 5. Efficacy of engineered bacterial strains in Balb-neuT tumor-burdened mice. This data demonstrates the anticancer therapeutic utility of strains BCT2, BCT5 and BCT14. All mice received 100 microliter injections of the indicated strains. Treatments (A) through (D) were previously described (11). All treated mice received 4 mg/kg VDA (vascular disrupting agent)+50 mg/kg CBD IP (cannabidiol) on Day −2. In treatment (E) on Day 0 mice received IV injections of 2×10⁶ cfu BCT2(pFF+20*-Quad)-2 hr-2×10⁶ cfu BCT2(pFF+20*-Quad), followed by 5×10⁶ cfu BCT5(pFF+20*-Quad)-2 hr-5×10⁶ cfu BCT5(pFF+20*-Quad) on Day 14. In treatment (F) mice received 2.5×10⁶ cfu BCT14-PL-Lux(pFliC-P)-2 hr-2.5×10⁶ cfu BCT14-PL-Lux(pFliC-P) on Day 0. Mice in cohorts (E) and (F) received 2 mg/kg VDA+50 mg/kg CBD IP two hours after the second IV injection of bacteria on Day 0. Statistics for tumor volume differences (Panel A) and mean survival time differences (Panel B) are provided only for those comparisons with p values<0.05.

FIG. 6. Plasmids that express and secrete immunomodulator proteins. Both the pFF+20*IL15Hly plasmid (A) and scFv secretion plasmids, represented by pFF+20*αCTLA4Hly (B) contain sequence from plasmid pYA292 including: the E. coli rrnB locus transcription terminator sequence, the p15A origin of replication, and cDNA coding for S. Typhimurium aspartate semi-aldehyde dehydrogenase. They also contain an FF+20* promoted operon consisting of an immunomodulator protein cDNA fused to the C-terminal 60 amino acids of the hemolysin secretion signal sequence (HlyA) and cDNAs coding for E. coli (HlyB) and (HlyD) hemolysin translocator proteins. The IL15 immunomodulator cDNA consists of sequence encoding the sushi domain of the mouse IL-15 receptor alpha subunit (IL15Ra), followed by a gly/ser flexible linker (L1), followed by sequence encoding mouse IL-15 (IL15), followed by sequence encoding a second gly/ser flexible linker (L2). The scFv immunomodulator cDNAs consist of sequence encoding the variable light (V1) and variable heavy (Vh) chain antibody sequences separated by a flexible gly/ser linker (L), followed by 6×Histidine and hemagglutinin tags (T). The strategy for constructing pFF+20*αPDL1 (not shown) was identical to pFF+20*αCTLA4.

FIG. 7. Western analysis of immunomodulator protein secretion. Soluble protein sampled from the media (M) and sonicated bacteria (B) from cultures of strain χ4550 harboring immunomodulator secretion plasmids were subjected to gel electrophoresis and transferred to PVDF membranes. All three membranes were probed with anti-DnaK antibody as a control for non-secreted protein. In panel A, a secondary antibody fused to a red fluorophore was used to identify DnaK, whereas DnaK was detected as a green fluorescent band in panels B and C. Proteins from the gel in panel A were probed with an anti-mouse IL-15 antibody, and the proteins from the gels in panels B and C were probed with an antibody to identify the six consecutive histidine residues located at the C-terminus of the anti-CTLA-4 or anti-PD-L1 scFv. The molecular weights of the protein ladder (Li-Cor cat. 928-40000) included in the gel in panel A (lane M.W.) are indicated.

FIG. 8. Treatment efficacy. BALB-neuT female mice with their largest tumor measuring <50 mm³ were either untreated or administered VDA, CBD and bacteria as described in Materials and Methods. Tumor sizes were measured for each mouse over the time course of the experiment, and the total tumor mass was compared to the tumor mass at the beginning of the experiment to calculate the fold change. Only the positive error bars representing one standard deviation are shown for clarity. Comparisons of Day 28 mean tumor burden using two-sample, two-tailed, unequal variance Student's t-tests resulted in the indicated P values. A vs B and B vs C were not considered significantly different (nd) because their P values were greater than 0.05.

FIG. 9. Kaplan-Meier survival analysis. The survival of mice from the efficacy experiment described in FIG. 8 was followed for 9 weeks post treatment. The mice were euthanized when one of their tumors exceeded 2 cm³. The mean survival time differences are reported with log-rank P values calculated using SAS JMP software version 15.1.0. A vs B and A vs C were not considered significantly different (nd) because their P values were greater than 0.05.

FIG. 10. Treatment toxicity. Changes in mouse weights from Day 0 are plotted to show the amount of toxicity by weight that mice experienced as a result of treatment. The maximum tolerated dose doxorubicin data (MTD DOXORUBICIN) were imported from a previously published study in tumor-burdened BALB-neuT mice [27]. Those mice were injected with 5 mg/kg doxorubicin, which is comparable to the maximum tolerated dose in humans by body surface area conversion.

### DETAILED DESCRIPTION OF THE INVENTION

Salmonella have a unique propensity to colonize solid tumors. Intravenous delivery of a significant amount of Salmonella yields a significantly higher tumor colonization rate yet the toxicity of the gram-negative bacteria precludes injection of a high number of bacteria. Provided herein are attenuated Salmonella genetically altered to become further attenuated to diminish the toxic side effects yet maintain their effectiveness to colonize tumors. In addition, provided herein are multiple genetically engineered Salmonella constructs that express and secrete various immunomodulating proteins that have significant anti-tumor effect. When given in therapeutic doses, these immunomodulating proteins can be toxic and the instant method of delivery reduces, and even eliminates, such toxicities.

Diminution of toxicity to levels allowing administration of the number of bacteria necessary for robust therapeutic colonization of autochthonous tumors has not been reported. The combination of flagellin, fimbriae, O-antigen, LPS and outer membrane protease mutations introduced into the bacterial strains allows nontoxic administration of sufficient numbers of bacteria to accomplish comprehensive robust colonization of the autochthonous tumors. Further, the systemic toxicity that limits the anticancer efficacy of administered immunomodulatory proteins is addressed by allowing simultaneous continuous secretion of multiple immunomodulator proteins directly into the tumor microenvironment, without the efficacy-limiting systemic toxicity of current cancer immunotherapy.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, several embodiments with regards to methods and materials are described herein. As used herein, each of the following terms has the meaning associated with it in this section.

For the purposes of clarity and a concise description, features can be described herein as part of the same or separate embodiments; however, it will be appreciated that the scope of the invention may include embodiments having combinations of all or some of the features described.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

As used herein, the indefinite articles “a”, “an” and “the” should be understood to include plural reference unless the context clearly indicates otherwise.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

As used herein, the term “about” means plus or minus 10% of the indicated value. For example, about 100 means from 90 to 110. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

The terms “individual,” “subject,” and “patient,” are used interchangeably herein and refer to any subject for whom diagnosis, treatment, or therapy is desired, including a mammal. Mammals include, but are not limited to, humans, farm animals, sport animals and pets. A “subject” is a vertebrate, such as a mammal, including a human. Mammals include, but are not limited to, humans, farm animals, sport animals and companion animals. Included in the term “animal” is dog, cat, fish, gerbil, guinea pig, hamster, horse, rabbit, swine, mouse, monkey (e.g., ape, gorilla, chimpanzee, orangutan) rat, sheep, goat, cow and bird.

The terms “treatment”, “treating” and the like are used herein to generally mean obtaining a desired pharmacologic and/or physiologic effect, such as arresting or inhibiting, or attempting to arrest or inhibit, the development or progression of a disorder and/or causing, or attempting to cause, the reduction, suppression, regression, or remission of a disorder and/or a symptom thereof. The effect may be prophylactic in terms of completely or partially preventing a disease or symptom thereof and/or may be therapeutic in terms of a partial or complete cure for a disease and/or adverse effect attributable to the disease. As would be understood by those skilled in the art, various clinical and scientific methodologies and assays may be used to assess the development or progression of a disorder, and similarly, various clinical and scientific methodologies and assays may be used to assess the reduction, regression, or remission of a disorder or its symptoms. Additionally, treatment can be applied to a subject or to a cell culture (in vivo or in vitro).

The terms “inhibit”, “inhibiting”, and “inhibition” refer to the slowing, halting, or reversing the growth or progression of a disease, infection, condition, group of cells, protein or its expression. The inhibition can be greater than about 20%, 40%, 60%, 80%, 90%, 95%, or 99%, for example, compared to the growth or progression that occurs in the absence of the treatment or contacting.

“Expression” refers to the production of RNA from DNA and/or the production of protein directed by genetic material (e.g., RNA (mRNA)). Inducible expression, as opposed to constitutive expression (expressed all the time), is expression which only occurs under certain conditions, such as in the presence of specific molecule (e.g., arabinose) or an environmental que.

The term “exogenous” as used herein with reference to a nucleic acid (or a protein) and a host refers to a nucleic acid that does not occur in (and cannot be obtained from) a cell of that particular type as it is found in nature or a protein encoded by such a nucleic acid. Thus, a nonnaturally-occurring nucleic acid is considered to be exogenous to a host once in the host. It is important to note that non-naturally occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally occurring nucleic acid, and thus is exogenous to a host cell once introduced into the host, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally occurring nucleic acid. It follows that genomic DNA fragments produced by PCR or restriction endonuclease treatment as well as cDNAs are considered to be non-naturally occurring nucleic acid since they exist as separate molecules not found in nature. An exogenous sequence may therefore be integrated into the genome of the host. It also follows that any nucleic acid containing a promoter sequence and polypeptide-encoding sequence (e.g., cDNA or genomic DNA) in an arrangement not found in nature is non-naturally occurring nucleic acid. A nucleic acid that is naturally occurring can be exogenous to a particular host microorganism. For example, an entire chromosome isolated from a cell of yeast x is an exogenous nucleic acid with respect to a cell of yeast y once that chromosome is introduced into a cell of yeast y.

In contrast, the term “endogenous” as used herein with reference to a nucleic acid (e.g., a gene) (or a protein) and a host refers to a nucleic acid (or protein) that does occur in (and can be obtained from) that particular host as it is found in nature. Moreover, a cell “endogenously expressing” a nucleic acid (or protein) expresses that nucleic acid (or protein) as does a host of the same particular type as it is found in nature. Moreover, a host “endogenously producing” or that “endogenously produces” a nucleic acid, protein, or other compound produces that nucleic acid, protein, or compound as does a host of the same particular type as it is found in nature.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” is an amount sufficient to effect beneficial or desired result, such as a preclinical or clinical result. An effective amount can be administered in one or more administrations. The term “effective amount,” as applied to the compound(s), biologics and pharmaceutical compositions described herein, means the quantity necessary to render the desired therapeutic result. For example, an effective amount is a level effective to treat, cure, or alleviate the symptoms of a disorder and/or disease for which the therapeutic compound, biologic or composition is being administered. Amounts effective for the particular therapeutic goal sought will depend upon a variety of factors including the disorder being treated and its severity and/or stage of development/progression; the bioavailability, and activity of the specific compound, biologic or pharmaceutical composition used; the route or method of administration and introduction site on the subject; the rate of clearance of the specific compound or biologic and other pharmacokinetic properties; the duration of treatment; inoculation regimen; drugs used in combination or coincident with the specific compound, biologic or composition; the age, body weight, sex, diet, physiology and general health of the subject being treated; and like factors well known to one of skill in the relevant scientific art. Some variation in dosage can occur depending upon the condition of the subject being treated, and the physician or other individual administering treatment will, in any event, determine the appropriate dose for an individual patient.

As used herein, “disorder” refers to a disorder, disease or condition, or other departure from healthy or normal biological activity, and the terms can be used interchangeably. The terms would refer to any condition that impairs normal function. The condition may be caused by sporadic or heritable genetic abnormalities. The condition may also be caused by non-genetic abnormalities. The condition may also be caused by injuries to a subject from environmental factors, such as, but not limited to, cutting, crushing, burning, piercing, stretching, shearing, injecting, or otherwise modifying a subject's cell(s), tissue(s), organ(s), system(s), or the like.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC5′ share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double stranded DNA and cDNA. Furthermore, the terms, “nucleic acid,” “DNA,” “RNA” and similar terms also include nucleic acid analogs, i.e., analogs having other than a phosphodiester backbone. For example, the so called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence, e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2× standard saline citrate (SSC), 0.1% SDS at 50° C.; preferably in 7% (SDS), 0.5 M NaPO4, 1 mM EDTA at 50° C. with washing in 1×SSC, 0.1% SDS at 50° C.; preferably 7% SDS, 0.5 M NaPO4, 1 mM EDTA at 50° C. with washing in 0.5×SSC, 0.1% SDS at 50° C.; and more preferably in 7% SDS, 0.5 M NaPO4, 1 mM EDTA at 50° C. with washing in 0.1×SSC, 0.1% SDS at 65° C. Suitable computer algorithms to determine substantial similarity between two nucleic acid sequences include GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, the term “pharmaceutically acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

The term “regulate” refers to either stimulating or inhibiting a function or activity of interest.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22: 1859-1862, 1981, and Matteucci et al., J. Am. Chem. Soc. 103:3185, 1981.

As used herein, the terms “including”, “includes”, “having”, “has”, “with”, or variants thereof, are intended to be inclusive similar to the term “comprising.”

The terms “comprises,” “comprising,” and the like can have the meaning ascribed to them in U.S. Patent Law and can mean “includes,” “including” and the like. As used herein, “including” or “includes” or the like means including, without limitation.

### Bacteria

Bacteria useful in the invention include, but are not limited to, Salmonella.

Examples of Salmonella strains which can be employed in the present invention include Salmonella typhi (ATCC No. 7251) and S. typhimurium (ATCC No. 13311). Attenuated Salmonella strains include S. typhi-aroC-aroD (Hone et al. Vacc. 9:810 (1991), S. typhimurium-aroA mutant (Mastroeni et al. Micro. Pathol. 13:477 (1992)) and Salmonella typhimurium 7207. Additional attenuated Salmonella strains that can be used in the invention include one or more other attenuating mutations such as (i) auxotrophic mutations, such as aro (Hoiseth et al. Nature, 291:238-239 (1981)), gua (McFarland et al Microbiol. Path., 3:129-141 (1987)), nad (Park et al. J. Bact, 170:3725-3730 (1988), thy (Nnalue et al. Infect. Immun., 55:955-962 (1987)), and asd (Curtiss, supra) mutations; (ii) mutations that inactivate global regulatory functions, such as cya (Curtiss et al. Infect. Immun., 55:3035-3043 (1987)), crp (Curtiss et al (1987), supra), phoP/phoQ (Groisman et al. Proc. Natl. Acad. Sci., USA, 86:7077-7081 (1989); and Miller et al. Proc. Natl. Acad. Sci., USA, 86:5054-5058 (1989)), phop.sup.c (Miller et al. J. Bact, 172:2485-2490 (1990)) or ompR (Dorman et al. Infect. Immun., 57:2136-2140 (1989)) mutations; (iii) mutations that modify the stress response, such as recA (Buchmeier et al. MoI. Micro., 7:933-936 (1993)), htrA (Johnson et al. MoI. Micro., 5:401-407 (1991)), htpR (Neidhardt et al. Biochem. Biophys. Res. Com., 100:894-900 (1981)), hsp (Neidhardt et al. Ann. Rev. Genet, 18:295-329 (1984)) and groEL (Buchmeier et al. Sci., 248:730-732 (1990)) mutations; mutations in specific virulence factors, such as IsyA (Libby et al. Proc. Natl. Acad. Sci., USA, 91:489-493 (1994)), pag or prg (Miller et al (1990), supra; and Miller et al (1989), supra), iscA or virG (d'Hauteville et al. MoI. Micro., 6:833-841 (1992)), plcA (Mengaud et al. Mol. Microbiol., 5:367-72 (1991); Camilli et al. J. Exp. Med, 173:751-754 (1991)), and act (Brundage et al. Proc. Natl. Acad. Sci., USA, 90:11890-11894 (1993)) mutations; (v) mutations that affect DNA topology, such as topA (Galan et al. Infect. Immun., 58: 1879-1885 (1990)); (vi) mutations that disrupt or modify the cell cycle, such as min (de Boer et al. Cell, 56:641-649 (1989)); (vii) introduction of a gene encoding a suicide system, such as sacB (Recorbet et al. App. Environ. Micro., 59:1361-1366 (1993); Quandt et al. Gene, 127:15-21 (1993)), nuc (Ahrenholtz et al. App. Environ. Micro., 60:3746-3751 (1994)), hok, gef, kil, or phlA (Molin et al. Ann. Rev. Microbiol., 47:139-166 (1993)); (viii) mutations that alter the biogenesis of lipopolysaccharide and/or lipid A, such as rFb (Raetz in Escherichia coli and Salmonella typhimurium, Neidhardt et al, Ed., ASM Press, Washington D.C. pp 1035-1063 (1996)), galE (Hone et al. J. Infect. Dis., 156:164-167 (1987)) and htrB (Raetz, supra), msbB (Reatz, supra; and U.S. Pat. No. 7,514,089); and (ix) introduction of a bacteriophage lysis system, such as lysogens encoded by P22 (Rennell et al. Virol, 143:280-289 (1985)), lamda murein transglycosylase (Bienkowska-Szewczyk et al. Mol. Gen. Genet., 184:111-114 (1981)) or S-gene (Reader et al. Virol, 43:623-628 (1971)).

The attenuating mutations can be either constitutively expressed or under the control of inducible promoters, such as the temperature sensitive heat shock family of promoters (Neidhardt et al. supra), or the anaerobically induced nirB promoter (Harbome et al. Mol. Micro., 6:2805-2813 (1992)) or repressible promoters, such as uapA (Gorfinkiel et al. J. Biol. Chem., 268:23376-23381 (1993)) or gcv (Stauffer et al. J. Bact, 176:6159-6164 (1994)).

In one embodiment, the strain of bacteria is VNP20009, a derivative strain of Salmonella typhimurium. Deletion of two of its genes—msbB and purI-resulted in its complete attenuation (by preventing toxic shock in animal hosts) and dependence on external sources of purine for survival. This dependence renders the organism incapable of replicating in normal tissue such as the liver or spleen, but still capable of growing in tumors where purine is available. In another embodiment, the strain of bacteria is SL3261 or χ11091.

### Vectors/Plasmids

In the present compositions and/or methods, DNA, RNA and/or protein may be produced by recombinant methods. The nucleic acid is inserted into a replicable vector for expression. Many such vectors are available. The vector components generally include, but are not limited to, one or more of the following: an origin of replication, one or more marker genes, an enhancer element, a promoter, and a transcription termination sequence and coding sequence. In some embodiments, for example in Salmonella, the gene and/or promoter (a sequence of interest) may be integrated into the host cell chromosome or may be presented on, for example, a plasmid/vector.

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors can contain a promoter that is recognized by the host organism and is operably linked to the nucleic acid sequence, such as a nucleic acid sequence coding for an open reading frame. Promoters are untranslated sequences located upstream (5′) to the start codon of a structural gene (generally within about 100 to 1000 bp) that control the transcription of particular nucleic acid sequence to which they are operably linked. In bacterial cells, the region controlling overall regulation can be referred to as the operator. Promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Promoters suitable for use with prokaryotic hosts include the β-lactamase and lactose promoter systems, alkaline phosphatase, a tryptophan (trp) promoter system, hybrid promoters such as the tac promoter, and starvation promoters (Matin, A. (1994) Recombinant DNA Technology II, Annals of New York Academy of Sciences, 722:277-291). However, other known bacterial promoters are also suitable. Such nucleotide sequences have been published, thereby enabling a skilled worker to operably ligate them to a DNA coding sequence. Promoters for use in bacterial systems also can contain a Shine-Dalgarno (S.D.) sequence operably linked to the coding sequence.

Construction of suitable vectors containing one or more of the above-listed components employs standard ligation techniques. Isolated plasmids or DNA fragments are cleaved, tailored, and re-ligated in the form desired to generate the plasmids required.

In some embodiments of the invention, the expression vector is a plasmid or bacteriophage vector suitable for use in Salmonella, and the DNA, RNA and/or protein is provided to a subject through expression by an engineered Salmonella (in one aspect attenuated) administered to the patient. The term “plasmid” as used herein refers to any nucleic acid encoding an expressible gene and includes linear or circular nucleic acids and double or single stranded nucleic acids. The nucleic acid can be DNA or RNA and may comprise modified nucleotides or ribonucleotides and may be chemically modified by such means as methylation or the inclusion of protecting groups or cap- or tail structures.

### Cancer Treatment

Bacteria such as Salmonella have a natural tropism for cancers, such as solid tumors. Types of cancer that can be treated using the methods of the invention include, but are not limited to, solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

In some aspects, the subject is treated with radiation, surgery and/or chemotherapy before, after or during administration of the bacterial cells described herein.

### Administration

The invention includes administration of the attenuated Salmonella strains described herein and methods for preparing pharmaceutical compositions and administering such as well. Such methods comprise formulating a pharmaceutically acceptable carrier with one or more of the attenuated Salmonella strains described herein.

A pharmaceutical composition of the invention is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

For intravenous administration, suitable carriers include physiological saline, bacteriostatic water, Cremophor EL™ (BASF; Parsippany, N.J.) or phosphate buffered saline (PBS). It must be stable under the conditions of manufacture and storage and must be preserved against the contaminating action of other (undesired) microorganisms. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Injectable solutions can be prepared by incorporating the active compound in the required amount in an appropriate solvent with one or a combination of ingredients discussed above. Generally, dispersions are prepared by incorporating the active compound into a vehicle which contains a basic dispersion medium and various other ingredients discussed above. In the case of powders for the preparation of injectable solutions, the preferred methods of preparation are vacuum drying and freeze-drying which yields a powder of the active ingredient plus any additional desired ingredient from a previously.

Oral compositions generally include an inert diluent or an edible carrier. For example, they can be enclosed in gelatin capsules. For the purpose of oral therapeutic administration, the active compound can be incorporated with excipients and used in the form of tablets, troches, or capsules.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the bacteria are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bacteria are formulated into ointments, salves, gels, or creams as generally known in the art.

It is especially advantageous to formulate compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention is dictated by and directly dependent on the unique characteristics of the active compound and the particular therapeutic effect to be achieved, and the limitations inherent in the art of compounding such an active compound for the treatment of individuals.

When administered to a patient the attenuated Salmonella can be used alone or may be combined with any physiological carrier. In general, the dosage ranges from about 1.0 c.f.u./kg to about 1×10¹² c.f.u./kg; optionally from about 1.0 c.f.u./kg to about 1×10¹⁰ c.f.u./kg; optionally from about 1.0 c.f.u./kg to about 1×10⁸ c.f.u./kg; optionally from about 1×10² c.f.u./kg to about 1×10⁸ c.f.u./kg; optionally from about 1×10⁴ c.f.u./kg to about 1×10⁸ c.f.u./kg; optionally from about 1×10⁵ c.f.u./kg to about 1×10¹² c.f.u./kg; optionally from about 1×10⁵ c.f.u./kg to about 1×10¹⁰ c.f.u./kg; optionally from about 1×10⁵ c.f.u./kg to about 1×10⁸ c.f.u./kg.

### EXAMPLES

The following examples are provided in order to demonstrate and further illustrate certain embodiments and aspects of the present invention and are not to be construed as limiting the scope thereof.

### Example I

Salmonella Enterica Typhimurium Engineered for Nontoxic Systemic Colonization of Autochthonous Tumors

### Introduction

In preclinical studies, strains of virulence-attenuated Salmonella enterica serovar Typhimurium (S. Typhimurium), genetically engineered to express numerous anticancer mechanisms, demonstrated efficacy (1). The preclinical studies have relied mostly on mouse models in which tumors develop from transplantation of preformed cancer tissue or injection of cancer cell lines. These tumors develop rapidly in rodents, requiring euthanasia within weeks of tumor development. Colonization of these tumors is robust, often reaching more than 1×10⁹ colony forming units (cfu) of bacteria per gram of tumor tissue (2).

Unfortunately, initial clinical trials of intravenous S. Typhimurium cancer therapy were disappointing, in part due to poor bacterial colonization of autochthonous (spontaneous) human tumors (3-5). In contrast to preclinical transplant tumor models, spontaneous tumors develop over months to years with more mature vasculature and limited necrotic space (6,7), which may account for the relatively poor colonization by bacteria seen in clinical trials. Although many attenuated strains of Salmonella have been used in preclinical bacterial cancer therapy research, none of them have been optimized for colonization of autochthonous tumors by systemic delivery. Almost all published studies of Salmonella cancer therapy have been in animal models involving tumors developed from transplanted cancer tissue or a bolus of cancer cells. Recent examples include: VNP20009 (8,9), A1-R (10,11), SL3261 (12,13) and v4550 (14,15). Also, many of these studies are necessarily carried out in immunodeficient animals. For example, experiments using Salmonella Typhimurium strain A1-R in patient-derived orthotopic xenograft mouse models (16) do not mimic colonization of autochthonous tumors and may not give an accurate assessment of the immune response in patients with an intact immune system.

Previous studies have shown that robust bacterial colonization of autochthonous tumors in genetically engineered mouse models (GEMM) can be improved by preconditioning the tumors with vasculature disrupting agents (VDA) to establish increased necrotic space (17). However, attempts to achieve consistent comprehensive colonization of VDA-conditioned autochthonous tumors by increased dosing of bacteria were limited by toxicity. To overcome this limitation, herein is provided an engineered strain of S. Typhimurium to be less immunogenic. Herein is described report the development of a strain of S. Typhimurium that has reduced systemic toxicity and is capable of robust colonization of autochthonous tumors conditioned with VDA.

### Materials and Methods

The characteristics of the plasmids are listed in Table 1. Plasmid pNG was constructed by EcoRI/HindIII digestion of plasmid pYA292 (18) and recircularization by ligation of the filled-in ends to remove expression of the LacZ-alpha peptide. Plasmid pLux was constructed by ligation of a PCR-generated fragment from pYA292 containing the LacUV5 promoter sequence, using primers LacFwd and LacRev, to the luxCDABE operon that was amplified by PCR from plasmid pAKlux2 (19) using primers LuxFwd and LuxRev. Plasmids pPflEPLux and pFF+20*Lux were constructed by replacing the LacUV5 promoter in plasmid pLux with tumor specific promoters PflEP (20) and FF+20*(21). gBlocks with PflEP and FF+20*sequences were digested with DraIII and BlpI. The resulting fragments were ligated to pLux that had been digested with DraIII and BlpI and treated with alkaline phosphatase. Restriction enzyme digestions, ligations and alkaline phosphatase treatments followed the manufacturer's guidelines (New England Biolabs, Ipswich, MA). Primers and gBlocks were obtained from Integrated DNA Technologies (Coralville, IA). See Table 2 for DNA sequences of all synthetic molecules used in plasmid construction.

TABLE 1
Bacterial strains and plasmids

| Strain or | Relevant | | Source or |
|---|---|---|---|
| plasmid | genotype | Origin | [reference] |
| Strain | | | |
| [symbol] | [symbol] | | [41] |
| [symbol] | [symbol] | | [42] |
| [symbol] | [symbol] | [symbol] | This study |
| [symbol] | [symbol] | | [24] |
| | [symbol] | | |
| BCT2 | [symbol] | [symbol] | This study |
| | [symbol] | | |
| | [symbol] | | |
| Plasmids | | | |
| [symbol] | [symbol] | | [symbol] |
| [symbol] | [symbol] | [symbol] | This study |
| [symbol] | [symbol] | | [symbol] |
| [symbol] | [symbol] | [symbol] | This study |
| | [symbol] | | |
| [symbol] | [symbol] | [symbol] | This study |
| [symbol] | [symbol] | [symbol] | This study |
| | [symbol] | | |

[symbol]

indicates data missing or illegible when filed

TABLE 2
Synthetic DNA molecules used for plasmid and strain construction.

| Molecule | Sequence |
|---|---|
| Cloning primers: | |
| LuxFwd | ATGACTAAAAAAATTTCATTCATTATTAACGGC |
| LuxRev | TTATTCAACTATCAAACGCTTCGGTTAAG |
| LacFwd | TTATTGGGCTGTTTTGGCGGAT |
| LacRev | TTATTGTTCCTCCTATAGTGAAATTGTTATCC |
| [symbol] | |
| [symbol] | CAACCTGTATCCGTCCGGCGTCATAAAAGGAAAAATAGAGGGAGGCATAATGATC |
| [symbol] | CGATAGCGATGAAAACGCGCCGAAGGATCATTATGCCTCCCTCTATTTTTCCTTT |
| [symbol] | CGCCGCCTAAGAAAAAGATCGGGGGAAGTGAAAAATTTTCTCCGGCGATTGATTC |
| [symbol] | CCTTGATTGTGTACCACGTGTCGGTGAATCAATCGCCGGAGAAAATTTTTCACTT |
| [symbol] | GAGGATTGCTTTATCAAAAACCTTCCAAAAGGAAAATTTTTTTATTTCGTTTTAT |
| [symbol] | AAAAGCCCCGAATTCACGGGGCTGAATAAAACGAAATAAAAAAATTTTCCTTTTG |
| [symbol] | AAGAGACTCTGTCTCATCCCAAACCTATTGTGGAGAAAAGAAAAACGCGCTGATA |
| [symbol] | GAAAACGCGCTGATACCGTAATAAGTATCAGCGCGTTTTTCTTTTCTCCACAATA |
| [symbol] | ATTCAGATGTAATCTGGAGAGGTGATGAAGTTTTCATCTT |
| [symbol] | TTCTCTGGCGTAATAAATGTACTCTTGTCCGACGATTTGATAAGATGAAAACTTCATCACCTCTCC |
| and detection primers by | |
| λ Red recombination: | |
| DSfwd | CGGCCTACAGAACCACACGCAGGCCCGATAAGCGCTGCAAATTGTGTAGGCTGGAGCTG |
| DSrev | CACAGGATACTGGCGCGCATACACAGCACATCTCTTTGCAGTCCATATGAATATCCTCCTTAGTTC |
| gBlocks: | |
| FF + 20° Lux gBlock | GGACGACGCAGATAGGATGCACAGTGTGCTGCGCCGTTCAGGTCAAAGAAGTGTCACTACCTGATG |
| | TT |
| | GAAGGATCAGGTAAATTTGATGTACATCAAATGGATCCAGATCGATGTTTAAAGATCCCCCCTCAC |
| | TCC |
| | TGCCATCATTCTGATATTGAAATAAGAGAGGAGGAACATAAATGACTAAAAAAATTTCATTCATTA |
| | TTAACGGCCAGGTTGAAATCTTTCC |
| | CGAAAGTGATGATTTAGTGCAATCCATTAATTTTG |
| | GTGATAATAGTGTTTACCTGCCAATATTGAATGACTCTCATGTAAAAAACATTATTGATATTGTAA |
| | TG |
| | GAAATAACGAATTACGGTTGCATAACATTGTCAATTTTC |
| | TCTATACGGTAGGGCAAAGATGGAAAAATGAAGAATACTCAAGACGCAGGACATACATTCGTGACT |
| | TA |
| | AAAAAATATATGGGATATTCAGAAGAAATGGCTAAGCTAGAGGCCAATTGGATATCT |
| PBEPLux gBlock | GGACGACGCAGATAGGATGCACAGTGTGCTGCGCCGTTCAGGTCAAAGAAGTGTCACTACCTGATG |
| | TT |
| | GAATTGGATAACGCCTCTTTGTCAGAACCTCTCCATTCGTTGACGCACATCAAGATAGCTTTCATT |
| | CGAA |
| | AGTAATTTAATCTTTATATGAAATAAGAGAGGAGGAACATAAATGACTAAAAAAATTTCATTCATT |
| | ATTA |
| | ACGGCCAGGTTGAAATCTTTCCCGAAAGTGATGATTTAGTGCAATCCATTAATTTTGGTGATAATA |
| | GTG |
| | TTTACCTGCCAATATTGAATGACTCTCATGTAAAAAACATTATATGATTGTAATGGAAATAACGAA |
| | TTACG |
| | GTTGCATAACATTGTCAATTTTCTCTATACGGTAGGGCAAAGATGAAAAATGAAGAATACTCAAGA |
| | CG |
| | CAGGACATACATTCGTGACTTAAAAAAATATATGGGATATTCAGAAGAAATGGCTAAGCTAGAGGC |
| | CAATTGGATATCT |

[symbol]

[symbol]

indicates data missing or illegible when filed

### Bacterial Strains

Bacterial strains, including relevant genotypes and sources are listed in Table 1. S. Typhimurium strain VNP20009 was obtained from the American Type Culture Collection (ATCC #202165). StCain L3261 was obtained from the Salmonella Genetic Stock Centre (SGSC #439). Stain BCT1 was constructed by deletion ofthe aspartate-semialdehyde dehydrogenase (asd) gene from the chromosome of SL3261 using the phage k Red recombinase method (22) and two PCR primers DSfwd and DSrev (Table 2). When measuring tumor colonization, the bacteria were transformed with plasmids that contain the lux operon (23) for bioluminescence. S. Typhimurium strain BCT2 was constructed by deleting the fliC, fljB, fimH, and rfaL genes from χ11091 (24), and introducing a single nucleotide change into the pgtE promoter in the χ11091 chromosome. Oligonucleotide primers used to introduce these mutations into χ11091 by the DIRex method (25) are listed in Table 2.

### Bacterial Growth Conditions

For experiments involving BCT1, the strain was transformed with plasmid pNG and bacterial injections were prepared from fresh mid-log phase lysogeny broth-Miller (LB) cultures. The cultures were harvested by centrifugation at 3,500×g for 5 min at 4° C. Cell pellets were resuspended in chilled phosphate buffered saline (PBS), pelleted and resuspended again at required concentrations. For experiments involving strains BCT2 or VNP20009, bacterial injections were prepared by growing cultures as indicated for strain BCT1(pNG), but the final cell resuspension was in chilled 20% glycerol/PBS (volume/volume) and cell samples were stored at −80° C. The frozen glycerol stocks were thawed and diluted into PBS to the desired concentration before use. After animal injection, the colony forming units (cfu) per ml of the injected bacteria was verified by dilution plating on LB agar at 37° C.

### Animal Experiments

BALB-neuT colony maintenance and husbandry was performed as previously described (26). The number of mice used for each data point is provided in figure captions. Tumors were measured by external caliper and tumor volumes were calculated as 0.5(length×width²). All agents were administered to mice parenterally by 100 microliter injections in PBS, at concentrations and dosing schedules indicated in individual experiments. Bacteria were injected into tail veins (IV). Vascular disruption agents (VDA) were injected either IV or intraperitoneally (IP), as indicated. VDA combretastatin A4 phosphate (CA4P; SF204, Selleck) was administered IV, and VDA CKD-516 (A07.020.548, Aurora Fine Chemicals) was administered IP. Anti-Interleukin-6 (IL-6) monoclonal antibodies (MP5-20F3, BioXCell) were injected IP. To measure the radiance of tumors in mice treated with luminescent bacteria, mice were anaesthetized by isoflurane inhalation at 3% in oxygen and imaged using an IVIS Spectrum in vivo imaging system with Living Image software (PerkinElmer). Total flux (luminescence) was acquired for 60 s and recorded as radiance (photons/sec/cm²/sr) for the tumors of interest.

### Abbreviations

S. Typhimurium: Salmonella enterica serovar Typhimurium; GEMM: genetically engineered mouse model; VDA: vascular disrupting agent; LB: lysogeny broth-Miller; PBS: phosphate-buffered saline; cfu: colony-forming units; IV: intravenous; IP: intraperitoneal; CA4P: combretastatin A-4 phosphate; IL-6: interleukin-6; CRS: cytokine release syndrome; PAMP: pathogen-associated molecular pattern.

### Results

### Removal of Intrinsic S. Typhimurium Toxicity

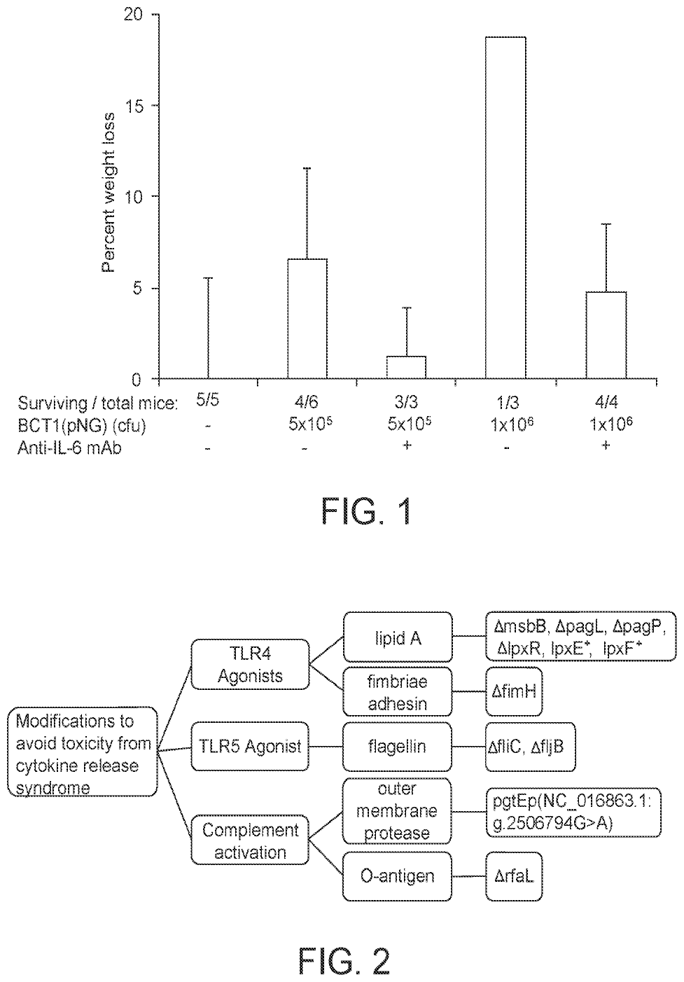

For estimation of maximum tolerated dose in human clinical trials, toxicology literature defines the upper limit for acute weight loss in short term (7 day) dosing studies of pharmaceuticals in rodents to be 10% (27). Suspecting that the toxicity observed upon IV administration of S. Typhimurium strain BCT1(pNG) with VDA might be due to cytokine release syndrome (CRS), a remedy reported to relieve toxicity due to CRS was applied that involved administration of antibodies antagonistic to interleukin-6 (IL-6) receptor activation (28). Mice treated with anti-IL-6 antibodies tolerated IV administration of 1×10⁶ cfu S. Typhimurium strain BCT1(pNG) plus a VDA with benign toxicity (<10% weight loss) compared to the unacceptable 18% weight loss and death of 2 of the 3 mice without anti-IL-6 antibodies (FIG. 1, columns 4 and 5). While administration of 5×10⁵ cfu bacteria was less toxic, a similar difference in percent weight loss was seen when comparing mice treated with or without anti-IL-6 antibodies (FIG. 1, columns 2 and 3).

When reduced toxicity due to blockage of IL-6 signaling was observed, the genes encoding S. Typhimurium surface molecules, known to induce IL-6 secretion from immune cells, were mutated to allow administration of increased amounts of bacteria with benign toxicity and avoid CRS. Beginning with S. Typhimurium χ11091 (24), a strain virulence-attenuated by folate auxotrophy and engineered for reduced lipopolysaccharide activation of toll-like receptor 4, genes encoding additional surface molecules responsible for induction of IL-6 secretion were deleted. Flagellin genes fliC and fljB were deleted (29), as was fimH, encoding the maltose receptor-binding adhesin subunit of fimbriae (30). In addition, 0-antigen was eliminated by deletion of rfaL (31,32). Finally, the transcriptional promoter sequence for pgtE was altered to increase expression of the PgtE outer membrane protease that inhibits complement activation (33). The resulting strain was named BCT2 (FIG. 2).

### Toxicity of Strain BCT2

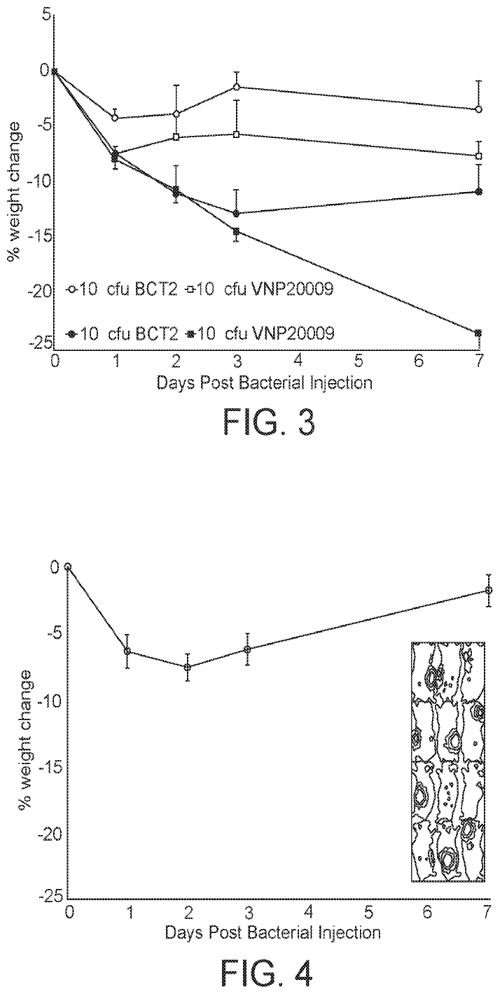

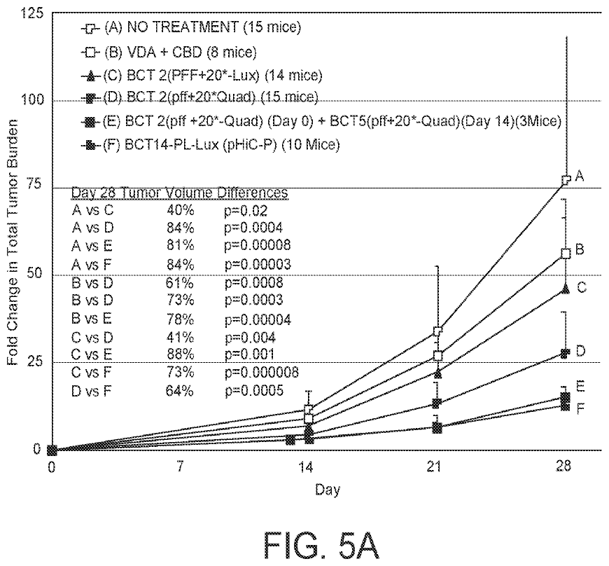

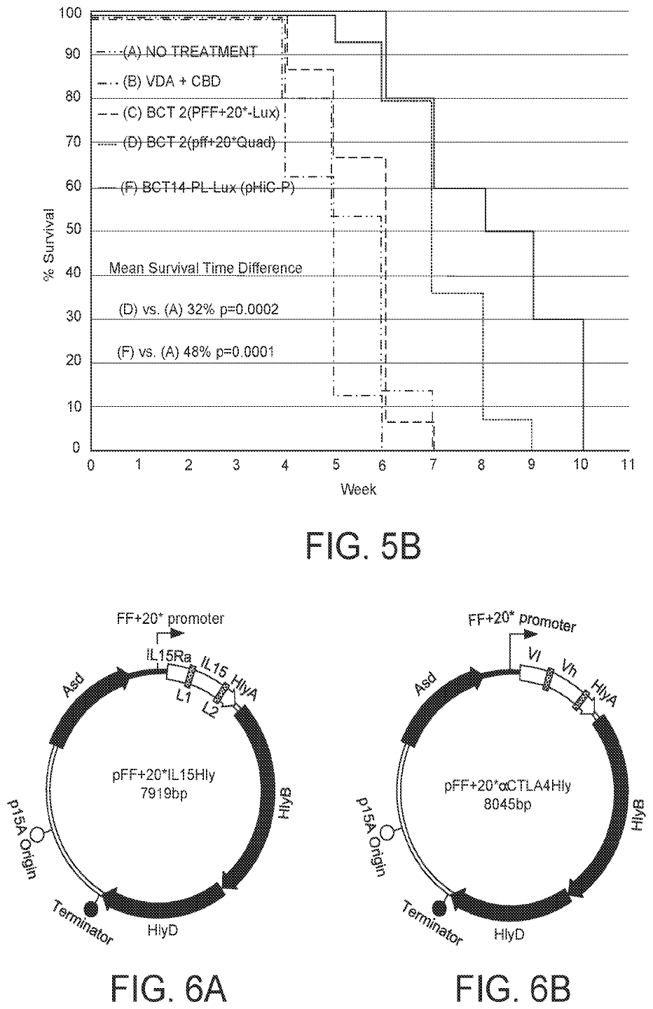

Non-tumor burdened mice were used to test the toxicity of strain BCT2 compared to strain VNP20009, a strain used in many preclinical studies and several clinical trials of S. Typhimurium cancer therapy (1,3-5). Treating non-tumor burdened mice with 1×10⁶ cfu of these strains resulted in less than 5% weight loss with BCT2(pPflEPLux) and less than 10% weight loss with VNP20009 (FIG. 3). The pPflEPLux plasmid was used in this experiment as a source of the asd gene to complement the chromosomal asd mutation, resulting in nonantibiotic balanced lethal plasmid maintenance (34). Treatment with 1×10⁷ cfu BCT2(pPflEPLux) resulted in about 12% weight loss after 3 days but recovery to about 10% weight loss after 7 days. Identical administration of 1×10⁷ cfu of strain VNP20009 resulted in 15% weight loss after 3 days and nearly 25% weight loss after 7 days (FIG. 3). These results indicate that strain BCT2 is significantly less toxic than strain VNP20009.

### Conditioning of Tumor Vasculature for Bacterial Colonization

When tumor-burdened mice were pre-treated with a VDA to increase the hypoxic necrotic space within tumors for colonization by facultative anaerobes such as S. Typhimurium, administration of 3×10⁶ cfu of strain BCT2(pFF+20*Lux) did not result in colonization of at least one tumor in every treated mouse (data not shown). The pFF+20*Lux plasmid contains the asd gene to complement the chromosomal asd mutation and an expressed lux operon to track the bacteria by whole body bioluminescence. Therefore, in addition to a VDA pre-treatment, post-bacteria administration of a VDA was added to ‘trap’ bacteria in the tumors. The toxicity and colonization of 3×10⁶ cfu of strain BCT2(pFF+20*Lux) administered on day 0 after a VDA given on days −4, −3, −2 and −1 was tested along with IP administration of a VDA one hour after pulsed injection of 3×10⁶ cfu bacteria. This protocol resulted in only benign toxicity and tumor colonization in all mice (FIG. 4).

### DISCUSSION

Bacteria have been employed for a number of medical applications but delivering bacteria to targeted tissues and avoiding the toxicity that results from an anti-bacterial immune response has been challenging. One application of bacterial therapy has been the development of Salmonella based strains as vaccine strains for treatment of salmonellosis in humans and other animals (35). In these cases, toxicity is a serious issue when the bacteria are administered systemically. Attempts to reduce the toxicity have focused on attenuating the immunogenicity of lipopolysaccharide endotoxin or flagella (35-37). By mutating the genes that code for lipid A synthesis as in strain χ11091 (FIG. 2), the toxicity of S. Typhimurium was reduced substantially.

In addition to vaccine strain development, another application of bacteria has been in the treatment of solid tumors (38). Specific to cancer therapy, the ‘holy grail’ of microbial-based cancer therapy is systemic delivery with robust colonization of solid tumors in a non-toxic manner (39). Bacteria used in these experiments include S. Typhimurium, Escherichia coli, and Clostridium perfringens, which can grow in the hypoxic, necrotic spaces of tumors. Preclinical investigation of bacterial anti-cancer potential has focused mostly on the use of S. Typhimurium in transplant tumor models in mice (38). In these experiments, tumor colonization is robust thus requiring fewer bacteria for treatment and consequently little to no toxicity. Very promising results with regard to efficacy were reported with several different types of solid tumors in these preclinical studies.

In an initial clinical trial of S. Typhimurium cancer therapy, a strain virulence-attenuated by amino acid auxotrophy and reduced lipopolysaccharide toxicity, VNP20009, was intravenously administered (4). The investigators reported a lack of efficacy and suggested that an increased bacterial dose would be needed to increase colonization of tumors, but toxicity of the bacteria would have to be overcome. The dose-limiting toxicity observed in that trial may have been due to overstimulation of a systemic immune response to the organism since most of its highly immunogenic Pathogen Associated Molecular Pattern (PAMP) molecules such as flagella and fimbriae remained intact (40). In spite of the successful application of the VNP20009 strain in preclinical trials using transplanted tumors, tumor colonization and toxicity were limiting in the clinical trials, which illustrates the difficulty in translating preclinical results to human trials.

In contrast to the transplant tumor models used in the preclinical studies, human tumors develop differently with regard to time, vasculature and necrotic space, which may account for the lack of translation from the preclinical mouse studies to the human trials. The use of an autochthonous tumor model, such as BALB-neuT mice that are immunocompetent, for preclinical studies is more relevant since it better resembles tumors that develop in humans. This is precisely why we have used an autochthonous model in our studies. However, when colonization of autochthonous tumors was used for comparison to transplant tumors in mice, several orders of magnitude less colonization was observed unless a VDA was used to increase the necrotic space of the tumors (17). Therefore, to achieve robust colonization of autochthonous tumors, the number of bacteria used for treatment and the amount of necrotic space within tumors have to be optimized and the resulting toxicities have to be addressed.

If S. Typhimurium is to be used to systemically target autochthonous tumors, induction of a toxic overstimulated systemic antibacterial immune response must be avoided. PAMP molecules on the surface of the bacteria will effectively stimulate an antitumor immune response in the tumor microenvironment but also will elicit a systemic immune response that results in unacceptable toxicity. Modifying the bacteria by disruption of genes that code for immune-stimulating PAMPs will allow their use as biological factories to secrete therapeutic molecules into the tumor microenvironment with minimal toxicity.

Addressing the toxicity caused by IV administered S. Typhimurium led to the construction of a genetically engineered organism, BCT2, with disruption of several genes involved in the expression of PAMPs (29-31) and a mutation to reduce activation of complement (33). When combined with a vascular disruption agent, this strain facilitates robust non-toxic colonization of autochthonous tumors in preclinical studies.

### BIBLIOGRAPHY

(1) Broadway K M, Scharf B E. Salmonella Typhimurium as an anticancer therapy: recent advances and perspectives. Curr Clin Micro Rpt. 2019; 6(4):225-239.
(2) Pawelek J M, Low K B, Bermudes D. Tumor-targeted Salmonella as a novel anticancer vector (Research Support, Non-U.S. Gov't). Cancer Res. 1997; 57(20):4537-4544.
(3) Nemunaitis J, Cunningham C, Senzer N, et al. Pilot trial of genetically modified, attenuated Salmonella expressing the E. coli cytosine deaminase gene in refractory cancer patients (Clinical Trial). Cancer Gene Ther. 2003; 10(10): 737-744.
(4) Toso J F, Gill V J, Hwu P, et al. Phase I study of the intravenous administration of attenuated Salmonella Typhimurium to patients with metastatic melanoma (Clinical Trial Clinical Trial, Phase I). J Cin Oncol. 2002; 20(1): 142-152.
(5) Heimann D M, Rosenberg S A. Continuous intravenous administration of live genetically modified Salmonella Typhimurium in patients with metastatic melanoma. J Immunother. 2003; 26(2):179-180.
(6) Falk P. Differences in vascular pattern between the spontaneous and the transplanted C3H mouse mammary carcinoma. Eur J Cancer Clin Oncol. 1982; 18(2):155-165.
(7) Lee J C, Kim D C, Gee M S, et al. Interleukin-12 inhibits angiogenesis and growth of transplanted but not in situ mouse mammary tumor virus-induced mammary carcinomas. Cancer Res. 2002; 62(3):747-755.
(8) Wang H, Chen T, Wan L, et al. Attenuated Salmonella engineered with an apoptosis-inducing factor (AIF) eukaryotic expressing system enhances its anti-tumor effect in melanoma in vitro and in vivo. Appl Microbiol Biotechnol. 2020; 104(8):3517-3528.
(9) Chen T, Zhao X, Ren Y, et al. Triptolide modulates tumour colonisation and anti-tumour effect of attenuated Salmonella encoding DNase I. Appl Microbiol Biotechnol. 2019; 103(2):929-939.
(10) Igarashi K, Kawaguchi K, Zhao M, et al. Recombinant methioninase combined with tumor-targeting Salmonella Typhimurium A1-R induced regression in a PDOX mouse model of doxorubicin-resistant dedifferentiated liposarcoma. Anticancer Res. 2020; 40(5):2515-2523.
(11) Igarashi K, Kawaguchi K, Zhao M, et al. Exquisite tumor targeting by salmonella A1-R in combination with caffeine and valproic acid regresses an adult pleomorphic rhabdomyosarcoma patient-derived orthotopic xenograft mouse model. Transl Oncol. 2020; 13(2):393-400.
(12) Mateos-Chavez A A, Munoz-Lopez P, Becerra-Baez E I, et al. Live attenuated Salmonella enterica expressing and releasing cell-permeable bax BH3 peptide through the MisL autotransporter system elicits antitumor activity in a murine xenograft model of human B non-hodgkin's lymphoma. Front Immunol. 2019; 10:2562.
(13) Jin C, Duan X, Liu Y, et al. T cell immunity induced by a bivalent Salmonella-based CEACAM6 and 4-1BBL vaccines in a rat colorectal cancer model. Oncol Lett. 2017; 13(5): 3753-3759.
(14) Sorenson B, Banton K, Frykman N, et al. Attenuated Salmonella Typhimurium with interleukin 2 gene prevents the establishment of pulmonary metastases in a model of osteosarcoma. J Pediatr Surg. 2008; 43(6):1153-1158.
(15) Sorenson B, Leonard Saltzman, et al. Antioxidant oils and Salmonella enterica Typhimurium reduce tumor in an experimental model of hepatic metastasis. Onco Targets Ther. 2011; 4:59-69.
(16) Murakami T, Hiroshima Y, Miyake K, et al. Efficacy of tumor targeting Salmonella Typhimurium A1-R against malignancies in patient-derived orthotopic xenograft (PDOX) murine models. Cells. 2019; 8(6):599.
(17) Drees J J, Mertensotto M J, Augustin L B, et al. Vasculature disruption enhances bacterial targeting of autochthonous tumors. J Cancer. 2015; 6(9):843-848.
(18) Galan J E, Nakayama K, Curtiss R, et al. Cloning and characterization of the asd gene of Salmonella Typhimurium: use in stable maintenance of recombinant plasmids in Salmonella vaccine strains. Gene. 1990; 94(1):29-35.
(19) Karsi A, Lawrence M L. Broad host range fluorescence and bioluminescence expression vectors for Gram-negative bacteria. Plasmid. 2007; 57(3):286-295.
(20) Leschner S, Deyneko I V, Lienenklaus S, et al. Identification of tumor-specific Salmonella Typhimurium promoters and their regulatory logic. Nucleic Acids Res. 2012; 40(7): 2984-2994.
(21) Ryan R M, Green J, Williams P J, et al. Bacterial delivery of a novel cytolysin to hypoxic areas of solid tumors (Evaluation Studies Research Support, Non-U.S. Gov't). Gene Ther. 2009; 16(3):329-339.
(22) Datsenko K A, Wanner B L. One-step inactivation of chromosomal genes in Escherichia coli K-12 using PCR products. Proc Natl Acad Sci USA. 2000; 97(12):6640-6645.
(23) Karsi A, Menanteau-Ledouble S, Lawrence M L. Development of bioluminescent Edwardsiella ictaluri for noninvasive disease monitoring. FEMS Microbiol Lett. 2006; 260(2):216-223.
(24) Kong Q, Six D A, Liu Q, et al. Phosphate groups of lipid A are essential for Salmonella enterica serovar Typhimurium virulence and affect innate and adaptive immunity. Infect Immun. 2012; 80(9):3215-3224.
(25) Nasvall J. Direct and Inverted Repeat stimulated excision (DIRex): simple, single-step, and scar-free mutagenesis of bacterial genes. PLoS One. 2017; 12(8):e0184126.
(26) Drees J, Mertensotto M, Liu G, et al. Attenuated Salmonella enterica Typhimurium reduces tumor burden in an autochthonous breast cancer model. Anticancer Res. 2015; 35(2): 843-849.
(27) Chapman K, Sewell F, Allais L, et al. A global pharmaceutical company initiative: an evidence-based approach to define the upper limit of body weight loss in short term toxicity studies. Regul Toxicol Pharmacol. 2013; 67(1):27-38.
(28) Maude S L, Barrett D, Teachey D T, et al. Managing cytokine release syndrome associated with novel T cell-engaging therapies. Cancer J. 2014; 20(2):119-122.
(29) Rolli J, Loukili N, Levrand S, et al. Bacterial flagellin elicits widespread innate immune defense mechanisms, apoptotic signaling, and a sepsis-like systemic inflammatory response in mice. Crit Care. 2010; 14(4): R160.
(30) Uchiya K I, Kamimura Y, Jusakon A, et al. Salmonella fimbrial protein FimH is involved in expression of proinflammatory cytokines in a toll-like receptor 4-dependent manner. Infect Immun. 2019; 87(3): e00881-18.
(31) Grossman N, Leive L. Complement activation via the alternative pathway by purified Salmonella lipopolysaccharide is affected by its structure but not its O-antigen length. J Immunol. 1984; 132(1):376-385.
(32) Kong Q, Yang J, Liu Q, et al. Effect of deletion of genes involved in lipopolysaccharide core and O-antigen synthesis on virulence and immunogenicity of Salmonella enterica serovar Typhimurium. Infect Immun. 2011; 79(10): 4227-4239.
(33) Hammarlof D L, Kroger C, Owen S V, et al. Role of a single noncoding nucleotide in the evolution of an epidemic African clade of Salmonella. Proc Natl Acad Sci USA. 2018; 115(11): E2614-E2623.
(34) Nakayama K, Kelly S M, Curtiss R. Construction of an Asd+ expression vector: stable maintenance and high expression of cloned genes in a Salmonella vaccine strain. Nat Biotechnol. 1988; 6(6):693-697.
(35) Galen J E, Buskirk A D, Tennant S M, et al. Live attenuated human salmonella vaccine candidates: tracking the pathogen in natural infection and stimulation of host immunity. EcoSal Plus. 2016; 7(1). DOI:10.1128/ecosalplus.ESP-0010-2016
(36) Low K B, Ittensohn M, Le T, et al. Lipid A mutant Salmonella with suppressed virulence and TNFalpha induction retain tumor-targeting in vivo. Nat Biotechnol. 1999; 17(1):37-41.
(37) Kong Q, Six D A, Liu Q, et al. Palmitoylation state impacts induction of innate and acquired immunity by the Salmonella enterica serovar Typhimurium msbB mutant. Infect Immun. 2011; 79(12):5027-5038.
(38) Mi Z, Feng Z C, Li C, et al. Salmonella-mediated cancer therapy: an innovative therapeutic strategy. J Cancer. 2019; 10(20):4765-4776.
(39) Forbes N S, Coffin R S, Deng L, et al. White paper on microbial anti-cancer therapy and prevention. J Immunother Cancer. 2018; 6; 6(1):78.
(40) de Jong H K, Parry C M, van der Poll T, et al. Host-pathogen interaction in invasive Salmonellosis. PLoS Pathog. 2012; 8(10): e1002933.
(41) Low K B, Ittensohn M, Luo X, et al. Construction of VNP20009: a novel, genetically stable antibiotic-sensitive strain of tumor-targeting Salmonella for parenteral administration in humans (Research Support, Non-U.S. Gov't). Methods Mol Med. 2004; 90:47-60.
(42) Hoiseth S K, Stocker B A. Aromatic-dependent Salmonella Typhimurium are non-virulent and effective as live vaccines. Nature. 1981; 291(5812):238-239.

### Example II

Virulence-Attenuated Salmonella Engineered to Secrete Immunomodulators Reduce Tumor Growth and Increase Survival in an Autochthonous Mouse Model of Breast Cancer

### Introduction

Until recently, the mainstays for cancer treatment included surgery, radiation and chemotherapy. A fourth treatment strategy, immunotherapy, is rapidly emerging and becoming widely adopted. However, stimulating the immune system to attack and guard against recurrence of cancer has been hindered by dangerous overactivation of a systemic immune response, which can result in unacceptable destruction of healthy tissue and in significant systemic toxicity [1]. Targeting molecules that modulate the immune system to the tumor microenvironment may avoid systemic toxicity and allow increased potential of anticancer immunotherapy to be achieved [2]. Herein is provided a virulence-attenuated strain of Salmonella enterica serovar Typhimurium (S. Typhimurium) for nontoxic colonization of tumors [118] and delivery of immunomodulators.

To demonstrate the therapeutic potential of this approach, provided herein is the combined delivery of a cytokine and two immune checkpoint inhibitors. The interleukin-15 superagonist IL-15Ra-IL-15 (RLI) [3] was chosen because of its toxicity-limited ability to provide strong anticancer efficacy [4], similar to the combination of anti-CTLA4 and anti-PD-L1 immune checkpoint inhibitors [5]. Furthermore, this therapy was tested in a model of autochthonous breast cancer rather than a transplantation cancer model to more stringently test its potential for successful clinical translation. Autochthonous tumor development allows more mature vascularization resulting in less necrotic space. Therefore, a vasculature disrupting agent (VDA) was included to increase necrotic space to allow administered bacteria to colonize [6]. To extend and maintain the VDA-induced necrotic space and reduce any acute bacterially induced systemic inflammatory response, cannabidiol (CBD) was included for its anti-angiogenic and anti-inflammatory properties [7, 8].

Disclosed herein is a report the anticancer efficacy of avirulent S. Typhimurium, which were engineered for targeted secretion of immunomodulators directly into the tumor microenvironment. These bacteria do not elicit the toxic responses inherent in cancer immunotherapy that result from systemic administration of immune stimulating molecules.

### Material and Methods

### Plasmids

The plasmids used in this study were designed to express and secrete various immunomodulators that could be tested for anti-cancer therapy (Table 1). The plasmids were constructed by replacing the Trc promoter and LacZalpha sequence in plasmid pYA292 [9] with an FF+20* [10] promoted operon. The FF+20* promoter was used for tumor-specific gene expression. The operon consisted of the FF+20* promoter, an immunomodulator cDNA sequence in frame with a C-terminal 60 amino acid E. coli HlyA secretion signal [111], which was followed by cDNA sequences coding for the E. coli hemolysin secretion proteins HlyB and HlyD (FIG. 6). Plasmids pFF+20*IL15Hly, pFF+20*αCTLA4Hly and pFF+20*αPDL1Hly, including their complete DNA sequences, were deposited at Addgene. The IL-15 sequence in pFF+20*IL15Hly is modeled from RLI [3]. The murine IL-15Ra sushi domain, isolated by PCR from plasmid pORF9-mIL15RAa (InvivoGen cat. porf-mil15raa), was joined through the 20 amino acid RLI flexible linker to cDNA coding for the murine IL-15 gene, which was isolated by PCR from plasmid pORF9-mIL15 (InvivoGen cat. porf-mil15). A second 18 amino acid glycine/serine-rich flexible linker, GQSSRSSGGGGSSGGGGS [12], was used to join the C-terminal amino acid of IL-15 to 60 amino acids at the C-terminus of the HlyA signal sequence. The anti-CTLA-4 and anti-PD-L1 scFv cDNA sequences, including C-terminal 6×Histidine and hemagglutinin tags, were isolated from immunized chicken antibody libraries as described previously [13]. They were joined at their C-terminal amino acid directly to the C-terminal 60 amino acid HlyA signal sequence. The E. coli hemolysin operon sequence in each of the immunomodulator plasmids was isolated by PCR from plasmid pNirB-PAop-hlyAs [14] using forward and reverse primers TTAGCCTATGGAAGTCAGGGTAATC and TTAACGCTCATGTAAACTTTCTGTTAC, respectively. For western analysis of immunomodulator expression and secretion, the FF+20* promoter and sequence immediately upstream from the consensus AGGAGG Shine-Dalgarno sequence were replaced by sequence containing the LacUV5 promoter [15] in pLacUV5IL15Hly AGATCTTCCGGAAGACCTTCCATTCTGAAATGAGCTGTTTACACTTTATGCTTCCGGCTCGTATAATGTGTGGAA TTGTGAGCGGATAACAATTTCACAAT, as well as the Trc promoter [16] in pTrcaCTLA4Hly and pTrcaPDL1Hly AGATCTTCCGGAAGACCTTCCATTCTGAAATGAGCTGTTGACAATTAATCATCCGGCTCGTATAATGTGTGGAAT TGTGAGCGGATAACAATTTCACAAT.

All plasmids contained the asd gene to complement the Dasd mutation in strains χ4550 and BCT2.

TABLE 1
Bacterial strains and plasmids.

| | | Source or |
|---|---|---|
| Strain or plasmid | Relevant genotype | reference |
| Strains | | |
| S. Typhimurium | | |
| _{χ}4550 | Δcrp-1, Δcya-1, ΔasdA1 | [17, 18] |
| BCT2 | ΔpabA1516, ΔpabB232 ΔasdA16, Δmsb848, | [19] |
| | ΔpagL7, ΔpagP81::P_{Ipp}IpxE, | |
| | ΔlpxR93::P_{Ipp}IpxF, ΔfimH, ΔfliC, ΔfljB, ΔrfaL, | |
| | pg(Ep(NC_016863.1:g.2506794G > A) | |
| Plasmids | | |
| pYA292 | asd | [9] |
| pFF + 20*Lux | pYA292 derivative containing an FF + 20* | [19] |
| | promoted lux operon | |
| pFF + 20*IL15Hly | pYA292 derivative containing an FF + 20* | This study |
| | promoted mouse IL15Rα-IL 15 gene with a | |
| | C-terminal hemolysin secretion signal and genes | |
| | for HlyB and HlyD. | |
| pFF + 20*αCTLA4Hly | pFF + 20*IL15Hly derivative with the IL15Rα-IL15 | This study |
| | sequence replaced by an anti-CTLA4 scFv | |
| | sequence. | |
| pFF+20*αPDL1Hly | pFF + 20*IL15Hly derivative with the IL15Rα-IL15 | This study |
| | sequence replaced by an anti-PDL1 scFv | |
| | sequence. | |
| pLacUV5ILI5Hly | pFF + 20*IL15Hly derivative with the FF + 20* | This study |
| | promoter and upstream untranslated sequence | |
| | replaced by sequence containing the LacUV5 | |
| | promoter. | |
| pTrcαCTLA4Hly | pFF + 20*αCTLA4Hly derivative with the FF + 20* | This study |
| | promoter and upstream unfr sequence replaced | |
| | by sequence containing the Trc promoter. | |
| pTrcαPDL1Hly | pFF + 20*αPDL 1Hly derivative with the FF + 20* | This study |
| | promoter and upstream untranslated sequence | |
| | replaced by sequence containing the Trc | |
| | promoter, | |

### Bacteria Strains

Bacterial strains, including relevant genotypes and sources, are listed in Table 1. Three strains were established to study expression and secretion of the three immunomodulator proteins by transforming strain χ4550 with pLacUV5IL15Hly, pTrcαCTLA4Hly or pTrcαPDL1Hly. Four strains were established for efficacy and toxicity experiments by transformation of strain BCT2 with plasmid pFF+20*Lux, pFF+20*IL15Hly, pFF+20*αCTLA4Hly or pFF+20*αPDL1Hly. Experiments were performed with either the single pFF+20*Lux transformed strain (BCT2pLux), or all four strains combined (BCT2pQuad), which included BCT2pLux to monitor tumor colonization.

### Analysis of Protein Expression

A single colony of strain χ4550 transformed with the immunomodulator plasmids was used to inoculate 50 ml of lysogeny broth-Miller (LB). The culture was grown with aeration at 37° C. for ˜17 hours to an optical density at 600 nm of ˜6.0. Twenty ml of the culture were harvested at 4000×g for 20 minutes at 4° C., and the culture medium was saved. The bacterial pellet was resuspended in phosphate buffered saline (PBS), recentrifuged, and resuspended in 2 ml PBS plus 1× Halt Protease Inhibitors (Thermo Fisher cat. 78430). The culture medium was vacuum filtered through a 0.2 μm filter, concentrated to about 400 μl by centrifugation through a 10 kDa cutoff Millipore Amicon centrifugal filter (Millipore, UFC901024) and then diluted to a final volume of 2 ml with PBS. The resuspended bacterial cells (0.5 ml of the original 2 ml) were sonicated on ice six times for 15 seconds at 40% power, using a Sonic Dismembrator (Dynatech Laboratories, Model 300), and centrifuged at 21,000×g for 12 minutes. Ten μl of 6× loading buffer (Boston Bioproducts cat. BP-111R) were added to 50 μl aliquots of the sonicate supernatant (representing soluble cellular proteins) and the concentrated culture medium, and the samples were incubated in a boiling water bath for 10 minutes. Each boiled sample (25 μl) was analyzed on a Tris-Tricine 10% to 20% gradient polyacrylamide gel. Proteins were electrophoretically transferred to a PVDF membrane and subjected to western analysis. The primary antibodies used in these experiments were: mouse anti-DnaK (Enzo Life Sciences cat. ADI-SPA-880), rat anti-mouse-IL15 (R&D Systems cat. MAB447) or mouse anti-His Tag (BioLegend cat. 65201). Secondary antibodies were goat anti-mouse (LI-COR IRDye 680RD cat. 926-68070) or goat anti-rat (LI-COR IRDye 800CW cat. 926-32219).

### Animal Experiments

For animal experiments, the bacterial cultures used for injections were prepared from fresh mid-log phase LB cultures of strain BCT2 containing either pFF+20*Lux, pFF+20*IL15Hly, pFF+20*αCTLA4Hly or pFF+20*αPDL1Hly. The cultures were harvested by centrifugation at 3,500×g for 5 minutes at 4° C. Cell pellets were resuspended in chilled phosphate buffered saline (PBS), pelleted, and then resuspended in chilled 20% glycerol/PBS (volume/volume). The cell samples were stored at −80° C. The frozen glycerol stocks were thawed and diluted into PBS to the desired concentration before use. Before storage at −80° C. and after animal injection, the colony forming units (cfu) per ml of the injected bacteria were verified by dilution plating on LB agar at 37° C.

BALB-neuT colony maintenance and husbandry were performed as previously described [20]. For determination of toxicity, mice were weighed and observed for reduced mobility and ruffled fur. Tumors were measured by external caliper, and tumor volumes were calculated as 0.5(length×width2). Mice were euthanized whenever a tumor exceeded 2 cm³. All agents were administered to mice parenterally by 100 μl injections in PBS, at concentrations and dosing schedules indicated in individual experiments. On day −2, mice were administered 4 mg/kg VDA (CKD-516, Aurora Fine Chemicals cat. A07.020.548) and 50 mg/kg CBD (99% pure crystal CBD, Endoca USA) in a single intraperitoneal injection. On day 0, mice were injected in a lateral tail vein (IV) with 1.5×10⁶ cfu bacteria, which was followed three hours later with another IV injection of 1.5×10⁶ cfu bacteria. This sequence was followed 1 hour later with an IP injection of 2 mg/kg VDA+50 mg/kg CBD. Mice were treated with BCT2pLux alone as a control or with BCT2pQuad. When BCT2pQuad was used, each of the four bacterial strains were mixed in equal amounts to achieve the 1.5×10⁶ cfu injection concentration.

To measure the radiance of tumors in mice treated with luminescent bacteria, mice were anesthetized by isoflurane inhalation at 3% in oxygen and imaged using an IVIS Spectrum in vivo imaging system with Living Image software (PerkinElmer). Total flux (luminescence) was acquired for 60 seconds and recorded as radiance (photons/sec/cm²/sr) for the tumors of interest.

### Abbreviations

S. Typhimurium: Salmonella enterica serovar Typhimurium; VDA: vascular disrupting agent; LB: lysogeny broth-Miller; PBS: phosphate-buffered saline; cfu: colony-forming units; IV: intravenous; IP: intraperitoneal; IL-15: interleukin-15; CBD: cannabidiol; MTD: maximum tolerated dose.

### Results

### Immunomodulator Secretion.

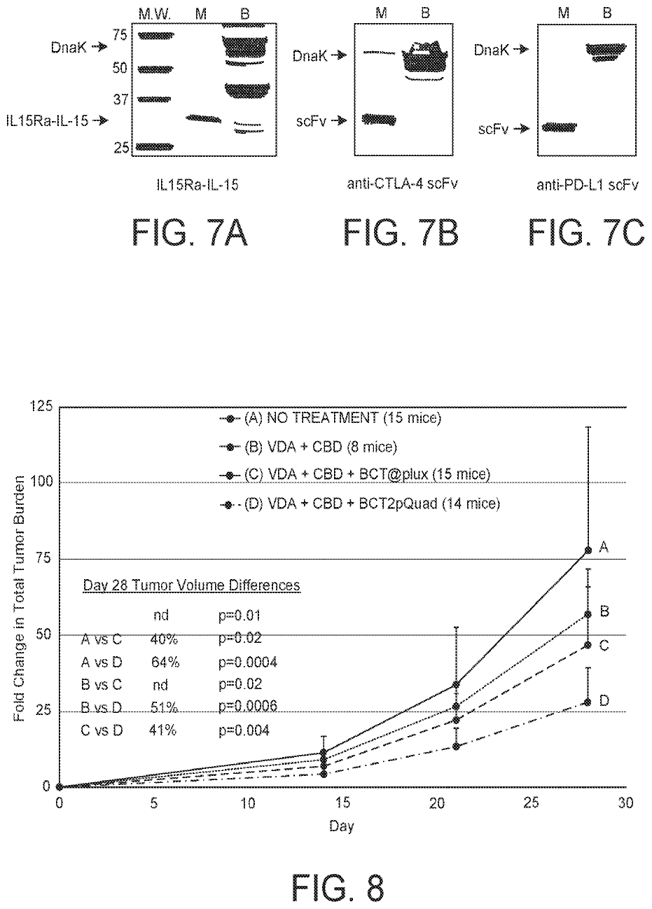

Secretion of immunomodulators through the hemolysin Type I secretion apparatus was tested. Western analysis of immunomodulator proteins expressed in S. Typhimurium strain χ4550 transformed with plasmids pLacUV5IL15Hly or pTrcαCTLA4Hly or pTrcαPD-L1Hly confirmed secretion of all three immunomodulators (FIG. 7). The presence of the intracellular DnaK protein in the bacteria sonicate (B) but not in the media (M) indicated that the immunomodulator protein bands observed in the media (M) were due to secretion rather than bacterial lysis. Furthermore, it was confirmed that these secreted immunomodulators were biologically active (data not shown).

Previously, it was observed that replacing the LacUV5 promoter, which constitutively promoted transcription of the lux operon, with the tumor-specific FF+20* promoter [10] resulted in tumor-specific expression of the lux operon [19]. Therefore, following confirmation of secretion of the immunomodulator proteins in vitro, the LacUV5 and Trc promoters in these plasmids were replaced with the FF+20* promoter. Confining expression of the immunomodulators to the tumor microenvironment was employed to avoid toxicity due the inflammatory stimulation of systemic immune cells, which may result in a cytokine release syndrome that often limits the achievable efficacy of cancer immunotherapy [21].

### Efficacy.

Four individual strains were established by transformation of strain BCT2 with pFF+20*Lux or with one of the plasmids encoding production of one of the three immunomodulator proteins. Tumor-burdened mice were treated with VDA two days prior to administration of bacteria to generate necrotic space for bacterial colonization. VDA also was administered one hour after the bacteria, which resulted in increased tumor colonization as shown in previous experiments (data not shown). On day −2, 4 mg/kg VDA was injected IP at a dose equal, by body surface area conversion, to the maximum tolerated dose (MTD) determined in human clinical trials [22]. On day 0, the mice were dosed with bacteria by two tail-vein injections separated by three hours. This “pulsed” administration of bacteria had been demonstrated to increase bacterial colonization of tumors, relative to injection of a single bolus dose [23], and this strategy was confirmed by experiments (data not shown). The VDA injected on day 0 was reduced to 50% MTD (2 mg/kg) due to toxicity observed when MTD VDA was administered after the bacteria (see below). To temporarily inhibit the acute angiogenesis that follows VDA-induced necrosis [24] and suppress any initial systemic inflammatory response to the bacteria, CBD was included in all VDA injections. The 50 mg/kg administered CBD dose is equivalent, by body surface area conversion, to the MTD determined in human clinical trials [25]. Bioluminescent imaging on days 4 or 5 revealed that in 10 of 15 BCT2pLux-injected and 9 of 14 BCT2pQuad-injected mice at least one tumor was colonized by bacteria (data not shown).

Growth of tumors in mice treated with VDA+CBD without bacteria, although trending toward slower growth, was not significantly different from tumor growth observed in nontreated mice (FIG. 8, Day 28, A vs B, P=0.1). When strain BCT2pLux was added to the treatment, growth of tumors by day 28 was significantly decreased by 40% relative to tumor growth in nontreated mice (FIG. 8, Day 28, A vs C, P=0.02). This suggests that BCT2 without immunomodulator expression may add a modicum of efficacy to this therapy, although a significant difference between VDA+CBD with and without BCT2pLux was not observed (FIG. 8, Day 28, B vs C, P=0.2). A significant decrease in tumor growth of 64%, relative to untreated animals, was observed in mice treated with BCT2pQuad (FIG. 8, A vs D, P=0.0004). The 41% and 51% additional slowing of tumor growth in mice injected with BCT2pQuad relative to that observed in mice injected with VDA+CBD with or without BCT2pLux, respectively (FIG. 8, Day 28, C vs D, P=0.004 or B vs D, P=0.0006), suggests that tumor-specific expression of the immunomodulators induces an antitumor immune response in addition to any antitumor efficacy provided by the bacteria alone.

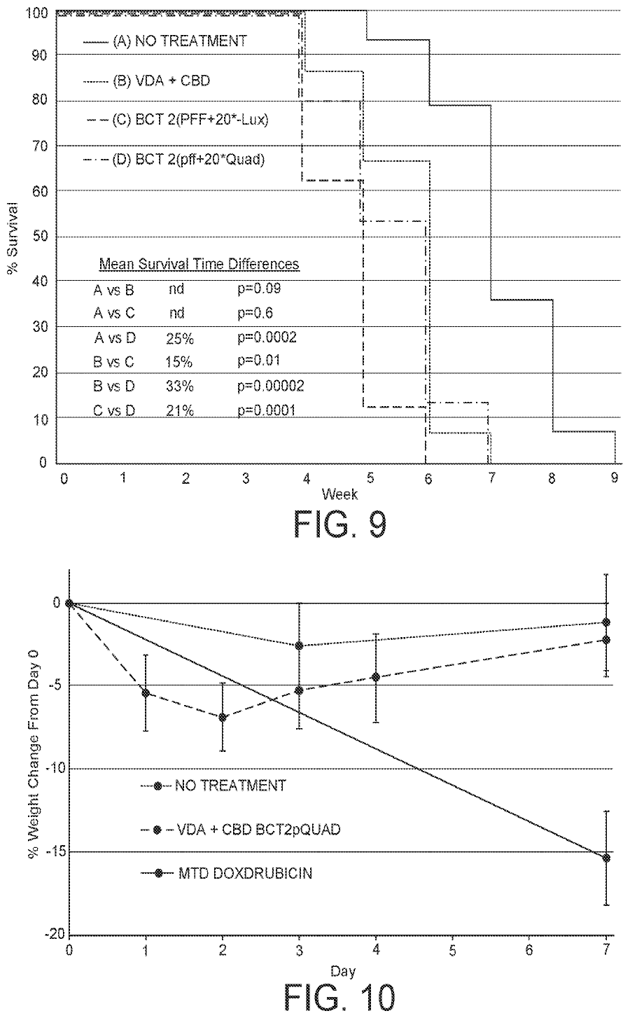

Mouse survival was followed for nine weeks following bacterial treatment (FIG. 9). Treatment with VDA+CBD alone trended toward a 12% reduction in survival time relative to untreated mice, albeit below 95% significance (FIG. 9, A vs B, P=0.09). This suggests a possible inhibition of anticancer immunity due to the anti-inflammatory property of CBD [8]. Adding BCT2pLux did not significantly affect survival relative to no treatment (FIG. 9, A vs C, P=0.6); however, survival relative to VDA+CBD alone increased by 15% (FIG. 9, B vs C, P=0.01). When BCT2pQuad was included in the treatment strategy, the mice survived 25% longer than nontreated mice (FIG. 9, A vs D, P=0.0002), 33% longer that mice treated only with VDA+CBD (FIG. 9, B vs D, P=0.00002) and 21% longer than when BCT2pLux is the only strain included in the treatment strategy (FIG. 9, C vs D, P=0.0001). This data further support the conclusion that the bacteria-delivered immunomodulators causes an efficacious anticancer immune response in the tumor microenvironment.

### Toxicity.

Adverse effects of treatment with VDA+CBD+BCT2pQuad were minimal. Toxicity, determined empirically by weight loss, reached a maximum of −6.9%+/−2.0% two days after BCT2pQuad administration (FIG. 10). Day 3 weights indicated the mice had resumed normal weight gain, relative to untreated animals. This result is well within the 7-day 10% weight loss considered to be the upper limit for translation of rodent-tested pharmaceuticals to human clinical trials [26]. In addition, this empirically determined benign toxicity was far less than that observed for the maximum tolerated dose chemotherapy (FIG. 10, MTD DOXORUBICIN). Mice injected with MTD doxorubicin continued weight loss to over 25% by day 14 and did not resume weight gain until day 21 (previously published data) [27]. Toxicity also was determined subjectively by observation of activity and coat smoothness on a scale of 0 to 3 as follows: 0) no difference in activity from untreated animals and a smooth shiny unruffled coat, 1) slightly less movement and a slightly ruffled coat, 2) obviously slowed movement and ruffled coat, and 3) no movement without coercion and a dull extremely ruffled coat. On this mobility/coat scale all of the BCT2pQuad injected mice were observed to be either 0/0, 1/0 or 0/1 on days 1 through 4, and all were scored as 0/0 on day 7. Therefore, although some level of toxicity was evident with this therapeutic strategy, it is of a benign nature and well within limits suitable for human clinical translation.

### DISCUSSION

Provided herein is a method to target delivery of multiple immunomodulator proteins to the tumor microenvironment, without the toxicity that has limited the potential efficacy of cancer immunotherapy. Previously reported studies using intravenous administration of bacteria have shown significant toxicities presumably due to the over-activation of the many inflammatory cascades elicited by Gram-negative bacteria (28, 29), which has subsequently limited the adoption of this treatment strategy. To address the toxicity problem, we have successfully developed a strain of S. Typhimurium that allows for the nontoxic intravenous administration of these bacteria (18) and have also engineered the bacteria for stealth and tumor-specific expression of immunomodulators. Importantly, these bacteria have demonstrated nontoxic anticancer efficacy in a mouse model of autochthonous breast cancer, which is more clinically relevant than models using transplant tumors (30).

Tumor colonization was enhanced by establishing necrotic space with VDA before and after administering the bacteria. Small molecule VDAs bind to tubulin and interfere with the cytoskeleton in immature vascular endothelial cells causing disruption of blood flow in tumors. This results in areas of hypoxia and ischemia, leading to the death of surrounding tumor cells and the formation of necrotic spaces (31). We believe that these created necrotic spaces provide the culture environment for these bacteria to thrive and subsequently release immunomodulators. Having demonstrated enhancement of S. Typhimurium colonization of autochthonous tumors using the VDA combretastatin [6], we used a more stable VDA, CKD-516 (32), to optimize dosing in the current study. We found that a single pretreatment with MTD VDA two days prior to administration of bacteria resulted in as much bacterial colonization of tumors as did dosing with VDA once a day for up to four days prior to administration of bacteria. This suggests that a single MTD of VDA may destroy most of the available premature intertumoral vasculature (33). Furthermore, inclusion of a VDA dose on day −1 reduced the amount of colonization (unpublished results), suggesting that VDA may interfere with the passage of bacteria from blood vessels into tumors if administered too close in time to bacterial treatment. The half-life of circulating CKD-516 is about 5 hours (34), so allowing two days for clearance of the VDA may facilitate bacterial colonization by allowing time for growth of nascent vasculature. After treating with the bacteria, it may be beneficial to wait two hours before dosing with VDA, rather than the one-hour interval used in the treatment strategy reported in this study, to increase tumor colonization by destroying the vasculature once again to potentially trap the bacteria in the tumor.

The anti-angiogenic and anti-inflammation properties of CBD were taken advantage of to temporarily inhibit angiogenesis, maintain necrotic space, and reduce acute toxicity due to bacterially stimulated systemic inflammation. Because VDA treatment is known to destroy vasculature and thereby stimulate an angiogenic response in tumors (35), it is possible that CBD, with its 24-hour half-life in circulation (36) and anti-angiogenic property (7) may extend the time of VDA induced necrosis, thus providing increased necrotic space for bacterial colonization of tumors.

The tumor microenvironment is a complex array of interacting cells and molecules that result in suppression of anticancer immunity [38]. While a number of monotherapies with single immunomodulators have demonstrated anticancer efficacy, combining immunomodulators in a single therapy is much more effective. For example, administering a combination of anti-PD-1 and anti-CTLA4 monoclonal antibodies resulted in patients surviving more than twice as long as when either treatment was administered alone [39]. Enhancing the therapy with additional immunomodulators to affect more cellular functions could further overcome the immunosuppressive nature of the tumor microenvironment and induce an increasingly activated antitumor immune response [40]. However, as is the case with all systemically delivered immunomodulators, toxicity due to immune-related adverse events limit the achievable efficacy of current anticancer immunotherapies, especially when immunomodulators are combined [41]. Overcoming the efficacy-limiting adverse effects of systemically delivered anticancer immunotherapy should allow delivery of additional combinations of immunomodulators beyond the IL-15 cytokine and anti-PD-L1 and anti-CTLA4 immune checkpoint inhibitors combined in this study. Our system of immunogenically stealth S. Typhimurium (BCT2) and tumor-specific expression of immunomodulators avoids systemic toxicity while maintaining significant efficacy in a mouse model of autochthonous breast cancer and increases the likelihood of successful clinical translation for treating cancer in humans.

### BIBLIOGRAPHY

1. Kennedy L B, Salama A K S. A review of cancer immunotherapy toxicity. CA Cancer J Clin. 2020; 70: 86-104.
2. Nguyen K B, Spranger S. Modulation of the immune microenvironment by tumor-intrinsic oncogenic signaling. J Cell Biol. 2020; 219.
3. Mortier E, Quemener A, Vusio P, et al. Soluble interleukin-15 receptor alpha (IL-15R alpha)-sushi as a selective and potent agonist of IL-15 action through IL-15R beta/gamma. Hyperagonist IL-15×IL-15R alpha fusion proteins. The Journal of biological chemistry. 2006; 281: 1612-1619.
4. Guo Y, Luan L, Rabacal W, et al. IL-15 Superagonist-Mediated Immunotoxicity: Role of NK Cells and IFN-gamma. J Immunol. 2015; 195: 2353-2364.
5. Martins F, Sofiya L, Sykiotis G P, et al. Adverse effects of immune-checkpoint inhibitors: epidemiology, management and surveillance. Nat Rev Clin Oncol. 2019; 16: 563-580.
6. Drees J J, Mertensotto M J, Augustin L B, Schottel J L, Saltzman D A. Vasculature Disruption Enhances Bacterial Targeting of Autochthonous Tumors. J Cancer. 2015; 6: 843-848.
7. Solinas M, Massi P, Cantelmo A R, et al. Cannabidiol inhibits angiogenesis by multiple mechanisms. British journal of pharmacology. 2012; 167: 1218-1231.
8. Atalay S, Jarocka-Karpowicz I, Skrzydlewska E. Antioxidative and Anti-Inflammatory Properties of Cannabidiol. Antioxidants (Basel). 2019; 9.
9. Galan J E, Nakayama K, Curtiss R, 3rd. Cloning and characterization of the asd gene of Salmonella typhimurium: use in stable maintenance of recombinant plasmids in Salmonella vaccine strains. Gene. 1990; 94: 29-35.
10. Ryan R M, Green J, Williams P J, et al. Bacterial delivery of a novel cytolysin to hypoxic areas of solid tumors. Gene therapy. 2009; 16: 329-339.
11. Hess J, Gentschev I, Goebel W, Jarchau T. Analysis of the haemolysin secretion system by PhoA-HlyA fusion proteins. Mol Gen Genet. 1990; 224: 201-208.
12. Finlay W J, Bloom L, Cunningham O. Optimized generation of high-affinity, high-specificity single-chain Fv antibodies from multiantigen immunized chickens. Methods in molecular biology. 2011; 681: 383-401.
13. Drees J J, Augustin L B, Mertensotto M J, Schottel J L, Leonard A S, Saltzman D A. Soluble production of a biologically active single-chain antibody against murine P D-L1 in Escherichia coli. Protein expression and purification. 2014; 94: 60-66.
14. Osorio M, Wu Y, Singh S, et al. Anthrax protective antigen delivered by Salmonella enterica serovar Typhi Ty21a protects mice from a lethal anthrax spore challenge. Infection and immunity. 2009; 77: 1475-1482.
15. Gilbert W. Starting and stopping sequences for the RNA polymerase. In: Losick R, Chamberlin M, editors. RNA Polymerase. Cold Spring Harbor, New York: Cold Spring Harbor Laboratory Press, 1976:193-205.
16. Brosius J, Erfle M, Storella J. Spacing of the −10 and −35 regions in the tac promoter. Effect on its in vivo activity. J Biol Chem. 1985; 260: 3539-3541.
17. Schodel F, Kelly S M, Peterson D L, Milich D R, Curtiss R, 3rd. Hybrid hepatitis B virus core-pre-S proteins synthesized in avirulent Salmonella typhimurium and Salmonella typhi for oral vaccination. Infect Immun. 1994; 62: 1669-1676.
18. Curtiss R, 3rd. Functional balanced-lethal host-vector systems. United States of America: University of Washington, St. Louis, MO, 2005.
19. Augustin L, Milbauer L, Hastings S, Leonard A, Saltzman D, Schottel J. Salmonella enterica Typhimurium Engineered for Nontoxic Systemic Colonization of Autochthonous Tumors. Manuscript submitted for publication. 2020.
20. Drees J, Mertensotto M, Liu G, et al. Attenuated Salmonella enterica Typhimurium Reduces Tumor Burden in an Autochthonous Breast Cancer Model. Anticancer Res. 2015; 35: 843-849.
21. Gutierrez C, McEvoy C, Munshi L, et al. Critical Care Management of Toxicities Associated With Targeted Agents and Immunotherapies for Cancer. Crit Care Med. 2020; 48: 10-21.
22. Oh D Y, Kim T M, Han S W, et al. Phase I Study of CKD-516, a Novel Vascular Disrupting Agent, in Patients with Advanced Solid Tumors. Cancer research and treatment: official journal of Korean Cancer Association. 2016; 48: 28-36.
23. Tome Y, Zhang Y, Momiyama M, et al. Primer dosing of S. typhimurium A1-R potentiates tumor-targeting and efficacy in immunocompetent mice. Anticancer Res. 2013; 33: 97-102.
24. Siemann D W, Chaplin D J, Horsman M R. Realizing the Potential of Vascular Targeted Therapy: The Rationale for Combining Vascular Disrupting Agents and Anti-Angiogenic Agents to Treat Cancer. Cancer Invest. 2017; 35: 519-534.
25. Bergamaschi M M, Queiroz R H, Zuardi A W, Crippa J A. Safety and side effects of cannabidiol, a Cannabis sativa constituent. Curr Drug Saf. 2011; 6: 237-249.
26. Chapman K, Sewell F, Allais L, et al. A global pharmaceutical company initiative: an evidence-based approach to define the upper limit of body weight loss in short term toxicity studies. Regul Toxicol Pharmacol. 2013; 67: 27-38.
27. Saltzman D, Augustin L, Leonard A, Mertensotto M, Schottel J. Low dose chemotherapy combined with attenuated Salmonella decreases tumor burden and is less toxic than high dose chemotherapy in an autochthonous murine model of breast cancer. Surgery. 2018; 163: 509-514.
28. Toso J F, Gill V J, Hwu P, et al. Phase I study of the intravenous administration of attenuated Salmonella typhimurium to patients with metastatic melanoma. Journal of clinical oncology: official journal of the American Society of Clinical Oncology. 2002; 20: 142-152.
29. Leschner S, Westphal K, Dietrich N, et al. Tumor invasion of Salmonella enterica serovar Typhimurium is accompanied by strong hemorrhage promoted by TNF-alpha. PLoS One. 2009; 4: e6692.
30. Guerin M V, Finisguerra V, Van den Eynde B J, Bercovici N, Trautmann A. Preclinical murine tumor models: a structural and functional perspective. Elife. 2020; 9.
31. Tozer G M, Kanthou C, Baguley B C. Disrupting tumour blood vessels. Nat Rev Cancer. 2005; 5: 423-435.
32. Lee J, Kim S J, Choi H, et al. Identification of CKD-516: a potent tubulin polymerization inhibitor with marked antitumor activity against murine and human solid tumors. J Med Chem. 2010; 53: 6337-6354.
33. Siemann D W. The unique characteristics of tumor vasculature and preclinical evidence for its selective disruption by Tumor-Vascular Disrupting Agents. Cancer Treat Rev. 2011; 37: 63-74.
34. Kim H K, Kang J W, Park Y W, et al. Phase I and pharmacokinetic study of the vascular-disrupting agent CKD-516 (NOV120401) in patients with refractory solid tumors. Pharmacol Res Perspect. 2020; 8: e00568.
35. Liang W, Ni Y, Chen F. Tumor resistance to vascular disrupting agents: mechanisms, imaging, and solutions. Oncotarget. 2016; 7: 15444-15459.
36. Millar S A, Stone N L, Yates A S, O'Sullivan S E. A Systematic Review on the Pharmacokinetics of Cannabidiol in Humans. Front Pharmacol. 2018; 9: 1365.
37. Kovalchuk O, Kovalchuk I. Cannabinoids as anticancer therapeutic agents. Cell Cycle. 2020; 19: 961-989.
38. Zou W. Immunosuppressive networks in the tumour environment and their therapeutic relevance. Nat Rev Cancer. 2005; 5: 263-274.
39. Larkin J, Chiarion-Sileni V, Gonzalez R, et al. Combined Nivolumab and Ipilimumab or Monotherapy in Untreated Melanoma. N Engl J Med. 2015; 373: 23-34.
40. Parayath N, Padmakumar S, Nair S V, Menon D, Amiji M M. Strategies for Targeting Cancer Immunotherapy Through Modulation of the Tumor Microenvironment. Regenerative Engineering and Translational Medicine. 2020; 6: 29-49.
41. Marshall H T, Djamgoz M B A. Immuno-Oncology: Emerging Targets and Combination Therapies. Front Oncol. 2018; 8: 315.

### Example III

### Plasmid Construction

### Hemolysin Secretion System

Plasmids were constructed by replacing the Trc promoter and LacZalpha sequence in plasmid pYA292 (1) with the FF+20* promoter sequence, which was used for tumor-specific gene expression (2). An operon consisted of the FF+20* promoter, an immunomodulator cDNA sequence in frame with a C-terminal 60 amino acid E. coli HlyA secretion signal (3), which was followed by cDNA sequences coding for the E. coli hemolysin secretion proteins HlyB and HlyD. The immunomodulatory cDNA sequences used for these constructs include: IL-15, αCTLA-4 scFv, and αPD-L1 scFv. The construction of plasmids containing these immunomodulator genes was described previously (4).

Using similar approaches (4), the cDNAs for the following immunomodulators were cloned:

*pFF+20*-CXCL9-10.

To construct pFF+20*-CXCL9-10, pFF+20*-IL15 and the CXCL9-10 gBlock were cleaved with BsrGI and Esp3I. The digested plasmid was treated with alkaline phosphatase and ligated to the digested gBlock. The ligation was used to transform strain χ6212, isolate plasmid DNA, and the plasmids were sequenced to verify the FF+20*-CXCL9-10 construct. The correct plasmid was used to transform strain χ3730A; plasmid was isolated from the transformants and used to transform strain BCT2.

CXCL9-10 gBlock sequence (1126 bp):
GATCCTTGTACATCAAATGGATCCAGATCGATGTTTAAAGATCCCCCCTC
ACTCCTGCCATCATTCTGATATTGAAATAAGAGAGGAGGAACATAAATGA
AGTCCGCTGTTCTTTTCCTCTTGGGCATCATCTTCCTGGAGCAGTGTGGA
GTTCGAGGAACCCTAGTGATAAGGAATGCACGATGCTCCTGCATCAGCAC
CAGCCGAGGCACGATCCACTACAAATCCCTCAAAGACCTCAAACAGTTTG
CCCCAAGCCCCAATTGCAACAAAACTGAAATCATTGCTACACTGAAGAAC
GGAGATCAAACCTGCCTAGATCCGGACTCGGCAAATGTGAAGAAGCTGAT
GAAAGAATGGGAAAAGAAGATCAGCCAAAAGAAAAAGCAAAAGAGGGGGA
AAAAACATCAAAAGAACATGAAAAACAGAAAACCCAAAACACCCCAAAGT
CGTCGTCGTTCAAGGAAGACTACAGTTCCCGGGGTAGGAGTTCCAGGTGT
TGGTGGGATCCCTCTCGCAAGGACGGTCCGCTGCAACTGCATCCATATCG
ATGACGGGCCAGTGAGAATGAGGGCCATAGGGAAGCTTGAAATCATCCCT
GCGAGCCTATCCTGCCCACGTGTTGAGATCATTGCCACGATGAAAAAGAA
TGATGAGCAGAGATGTCTGAATCCGGAATCTAAGACCATCAAGAATTTAA
TGAAAGCGTTTAGCCAAAAAAGGTCTAAAAGGGCTCCTGGTCAGTCCTCT
AGATCTTCCGGCGGTGGTGGCAGCTCCGGTGGTGGCGGTTCCTTAGCCTA
TGGAAGTCAGGGTAATCTTAATCCATTAATTAATGAAATCAGCAAAATCA
TTTCAGCTGCAGGTAATTTTGATGTTAAAGAGGAAAGAGCTGCAGCTTCT
TTATTGCAGTTGTCCGGTAATGCCAGTGATTTTTCATATGGACGGAACTC
AATAACTTTGACAGCATCAGCATAATTTATTAATTTAAATAATAGCAATC
TTACTGGGCTGTGCCACATAAGATTGCTATTTTTTTGGAGTCATAATGGA
TTCTTGTCATAAAATTGATTATGGGTTATACGCCCTGGAGATTTTAGCCC
AATACCATAACGTCTCTGTTAACCCG

*pFF+20*-αPD-L1N (N=nanobody)

To construct pFF+20*-αPD-L1N, pFF+20*-IL15 and the PDL1N gBlock were cleaved with BamHI and PacI. The digested plasmid was treated with alkaline phosphatase and ligated to the digested gBlock. The ligation was used to transform strain χ6212, isolate plasmid DNA, and the plasmids were sequenced to verify the FF+20*-αPD-L1N construct. The correct plasmid was used to transform strain χ3730A; plasmid was isolated from the transformants and used to transform strain BCT2.

PDLI1 gBlock sequence (557 bp):
TCAAATGGATCCAGATCGATGTTTAAAGATCCCCCCTCACTCCTGCCATC
ATTCTGATATTGAAATAAGAGAGGAGGAACATAAATGGCTCAAGTTCAAT
TAGTAGAAACAGGGGGAGGCTTAGTTCAGCCTGGAGGATCTCTTCGCTTA
TCTTGCACTGCTTCAGGCTTCACATTCTCGATGCACGCGATGACCTGGTA
CCGTCAAGCGCCGGGGAAGCAGCGTGAATTGGTGGCAGTTATTACGAGTC
ATGGGGACCGGGCGAACTACACTGATAGCGTCCGTGGGCGTTTCACGATT
AGCCGCGACAATACCAAAAACATGGTATACTTACAGATGAATAGCTTAAA
ACCGGAAGATACGGCTGTGTATTACTGTAACGTTCCACGGTACGATTCAT
GGGGGCAGGGTACTCAAGTCACTGTCAGCAGTGGCGGCTTACCGGAAACA
GGGGGTCACCACCACCATCATCACGGTGCGTACCCCTATGATGTGCCTGA
CTATGCGAGCTTAGCCTATGGAAGTCAGGGTAATCTTAATCCATTAATTA
ACCTCTC

### Flagellar Secretion System

To utilize the flagellar secretion system (5), plasmids were constructed to contain the FliC promoter sequence and a FliC secretion signal fused to immunomodulatory DNA sequences. The activity of the plasmid FliC promoter is controlled by the chromosomal flagellar locus, which is driven by the tumor-specific FF+20* promoter (2) in order to confine immunomodulator expression and secretion to the tumor microenvironment. See the description of strain BCT14.

The immunomodulatory cDNA sequences used for these constructs include:

*pFliC-IL-15

To make pFliC-IL15, the FLIC-IL15 gBlock-2 and plasmid pLacUV5-mIL15Ra-mIL15 (4) were digested with DraIII and SphI. The digested plasmid was treated with alkaline phosphatase and ligated to the digested gBlock. The ligation was used to transform strain χ6212, isolate plasmid DNA, and the plasmids were sequenced to verify the FliC-IL15 construct. The correct plasmid was used to transform strain χ3730A; plasmid was isolated from the transformants and used to transform strain BCT13.

FLIC-IL15 gBlock-2 sequence (380 bp):
CCAGTCCACAGTGTGCGGAATAATGATGCATAAAGCGGCTATTTCGCCGC
CTAAGAAAAAGATCGGGGGAAGTGAAAAATTTTCTAAAGTTCGAAATTCA
GGTGCCGATACAAGGGTTACGGTGAGAAACCGTGGGCAACAGCCCAATAA
CATCAAGTTGTAATTGATAAGGAAAAGATCATGGCACAAGTCATTAATAC
AAACAGCCTGTCGCTGTTGACCCAGAATAACCTGAACAAATCCCAGTCCG
CTCTGGGCACCGCTATCGAGCGTCTGTCTTCCGGTCTGCGTATCAACAGC
GCGAAAGACGATGCGGCAGGTCCGCTGGGCCTGGCGGGAACCACGTGTCC
ACCTCCCGTATCTATTGAGCATGCTTCCAG

The construction of the pFliC plasmids with the following immunomodulator genes followed a protocol similar to the construction of pFliC-IL15.

*pFliC-αPD-L1 scFv

To make pFliC-αPD-L1 scFv, the pLacUV5-OmpA-αPD-L1 plasmid and the αPD-L1 gBlock were digested with BstEII and AvrII. The digested plasmid was treated with alkaline phosphatase and ligated to the digested gBlock. The ligation was used to transform strain χ6212, isolate plasmid DNA, and the plasmids were sequenced to verify the FliC-αPD-L1 construct. The correct plasmid was used to transform strain χ3730A; plasmid was isolated from the transformants and used to transform strain BCT13.

FLIC-αPDL1 gBlock (1053 bp):
GACTGAGGTGACCGGCACGTTGACTACGCCGGTTGGTCGTCTGCGTAAGC
TGAACATGGGGCCAGAGTTCTTGTCGGCGTTTACCGTAGGCGACCAGTTG
TTATGGGGCGCCGCCGAGCCGCTGCGTCGAATGCTGCGCCAGTTGGCGTA
GTGGCTATTGCAGCGCTTATCGGGCCTGCGTGTGGTTCTGTAGGCCGGAT
AAGGCGCGTCAGCGCCGCCATCCGGCGGGGAAATTTGTGTTAAACCAGGG
GTGCATCGTCACCCTTTTTTTGCGTAATACAGGAGTAAACGCAGATGTTT
CATTTTTATCAGGAGTTAAGCAGAGCATTGGCTATTCTTTAAGGGTAGCT
TAATCCCACGGGTATTAAGCCTAACCTGAAGGTAGGACGACGCAGATAGG
ATGCACAGTGTGCGGAATAATGATGCATAAAGCGGCTATTTCGCCGCCTA
AGAAAAAGATCGGGGGAAGTGAAAAATTTTCTAAAGTTCGAAATTCAGGT
GCCGATACAAGGGTTACGGTGAGAAACCGTGGGCAACAGCCCAATAACAT
CAAGTTGTAATTGATAAGGAAAAGATCATGGCACAAGTCATTAATACAAA
CAGCCTGTCGCTGTTGACCCAGAATAACCTGAACAAATCCCAGTCCGCTC
TGGGCACCGCTATCGAGCGTCTGTCTTCCGGTCTGCGTATCAACAGCGCG
AAAGACGATGCGGCAGGTCCGCTGGGCCTGGCGGGACTGACTCAGCCGTC
CTCGGTGTCAGCAAACCTGGGAGGAACCGTCAAGATCACCTGCTCCGGGG
GTAGTGGCAGCTACGGCTGGTATCAGCAGAAGGCACCTGGCAGTGCCCCT
GTCAGTCTGATCTATGACAACACCAACAGACCCTCGGACATCCCTTCACG
ATTCTCCGGTGCCCTATCCGGCTCCACAGCCACATTAACCATCACTGGGG
TCCAAGCCGAGGACGAGGCTGTCTATTACTGTGGGAGCAGGGACAGCAGT
AATGCTGGTTCTGTATTTGGGGCCGGGACAACCCTGACCGTCCTAGGTCA
GTC

*pFliC-αCTLA-4 scFv

To make pFliC-αCTLA-4 scFv, the pFliC-αPD-L1 scFv plasmid and αCTLA4 gBlock were digested with NsiI and SpeI. The digested plasmid was treated with alkaline phosphatase and ligated to the digested gBlock. The ligation was used to transform strain χ6212, isolate plasmid DNA, and the plasmids were sequenced to verify the FliC-αCTLA4 construct. The correct plasmid was used to transform strain χ3730A; plasmid was isolated from the transformants and used to transform strain BCT13.

FLIC-αCTLA4 gBlock (1070 bp):
TGGTTGATGCATAAAGCGGCTATTTCGCCGCCTAAGAAAAAGATCGGGGG
AAGTGAAAAATTTTCTAAAGTTCGAAATTCAGGTGCCGATACAAGGGTTA
CGGTGAGAAACCGTGGGCAACAGCCCAATAACATCAAGTTGTAATTGATA
AGGAAAAGATCATGGCACAAGTCATTAATACAAACAGCCTGTCGCTGTTG
ACCCAGAATAACCTGAACAAATCCCAGTCCGCTCTGGGCACCGCTATCGA
GCGTCTGTCTTCCGGTCTGCGTATCAACAGCGCGAAAGACGATGCGGCAG
GTCCGCTGGGCCTGGCGGGACTGACTCAGCCGTCCTCGGTGTCAGCGAAC
CCGGGAGAAACCGTCGAGATCACCTGCTCCGGGGGTAGCAACTACTATGG
CTGGTACCAGCAGAAGTCACCTGGCAGTGCCCCTGTCACTCTGATCTATA
GCAGCACCAACAGACCCTCAAACATCCCTTCACGATTCTCCGGTTCCAAA
TCCGGCTCCACACACACATTAACCATCACTGGGGTCCAAGCCGACGACGA
GGCTGTCTATTACTGTGGGAGTGTAGACAGCAGCTATGTTGGTGTATTTG
GGGCCGGGACAACCCTGACCGTCCTAGGTCAGTCCTCTAGATCTTCCGGC
GGTGGTGGCAGCTCCGGTGGTGGCGGTTCCGCCCTGACGTTGGACGAGTC
CGGGGGCGGCCTCCAGACACCGAAAGGAGGGCTCAGCCTCGTCTGCAAGG
CCTCCGGGTTCACCTTCAGCAGCTTCAACATCTACTGGGTGCGACAGGCG
CCCGGCAAGGGGCTGGAATACGTCGCTGAAATTAGCGGCACTGGCAGTTA
CACAAACTACGGGCCGGCGGTGGATGGCCGTGCCACCATCTCGAGGGACA
ACGGGCAGAGCACAGTGAGGCTGCAGCTGAGCAATCTCAGGGCAGAGGAC
ACCGCCACCTACTACTGCGCCAAAGCTGCTGGCGGTTGTAGTGGTTATGG
TTGGTGTCCTGCTGGCATCGACGCATGGGGCCACGGGACCGAAGTCATCG
TCTCCTCCACTAGTTGCTAG

*pFliC-CXCL9-10

To make pFliC-CXCL9-10, the pFliC-IL15 plasmid and the CXCL9-10 pFlic gBlock were digested with HindIII and DraIII. The digested plasmid was treated with alkaline phosphatase and ligated to the digested gBlock. The ligation was used to transform strain χ6212, isolate plasmid DNA, and the plasmids were sequenced to verify the FliC-CXCL9-10 construct. The correct plasmid was used to transform strain χ3730A; plasmid was isolated from the transformants and used to transform strain BCT13.

CXCL9-10 pFlic gBlock (991 bp):
CCAGTCCACAGTGTGCGGAATAATGATGCATAAAGCGGCTATTTCGCCGC
CTAAGAAAAAGATCGGGGGAAGTGAAAAATTTTCTAAAGTTCGAAATTCA
GGTGCCGATACAAGGGTTACGGTGAGAAACCGTGGGCAACAGCCCAATAA
CATCAAGTTGTAATTGATAAGGAAAAGATCATGGCACAAGTCATTAATAC
AAACAGCCTGTCGCTGTTGACCCAGAATAACCTGAACAAATCCCAGTCCG
CTCTGGGCACCGCTATCGAGCGTCTGTCTTCCGGTCTGCGTATCAACAGC
GCGAAAGACGATGCGGCAGGTCCGTTAGGACTTGCAGGCAAATCCGCCGT
TTTATTTTTACTGGGGATTATCTTTCTGGAGCAATGCGGCGTACGCGGGA
CCCTGGTCATCCGCAACGCACGCTGCTCGTGCATCTCTACTTCCCGGGGA
ACAATTCATTATAAGTCCTTGAAGGACTTGAAGCAGTTTGCGCCGTCACC
TAACTGCAATAAGACTGAGATTATTGCAACCCTTAAAAATGGGGACCAAA
CCTGCTTGGACCCTGACAGCGCAAATGTCAAGAAACTGATGAAAGAATGG
GAAAAGAAAATTTCCCAGAAAAAGAAACAAAAACGGGGTAAGAAGCATCA
GAAAAATATGAAAAATCGTAAACCAAAGACCCCACAGAGCCGTCGGCGTA
GTCGTAAAACGACAGTGCCAGGCGTTGGTGTACCGGGAGTCGGGGGCATC
CCATTAGCCCGGACGGTTCGGTGCAACTGCATTCACATCGACGATGGCCC
TGTACGTATGCGGGCCATTGGAAAATTGGAAATCATCCCAGCCAGTCTTT
CCTGCCCACGGGTTGAAATCATTGCGACTATGAAGAAGAATGATGAGCAA
CGTTGCCTTAACCCGGAATCCAAAACTATCAAAAACCTGATGAAGGCTTT
CAGTCAAAAACGTTCTAAGCGGGCACCTTAAGCTTGACACG

*pFliC-αCTLA-4N (N=nanobody)

To make pFliC-αCTLA-4N, the pFliC-IL15 plasmid and the αCTLA4N pFliC gBlock were digested with HindIII and DraIII. The digested plasmid was treated with alkaline phosphatase and ligated to the digested gBlock. The ligation was used to transform strain χ6212, isolate plasmid DNA, and the plasmids were sequenced to verify the FliC-αCTLA-4N construct. The correct plasmid was used to transform strain χ3730A; plasmid was isolated from the transformants and used to transform strain BCT13.

αCTLA4N pFliC gBlock (727 bp):
CCAGTCCACAGTGTGCGGAATAATGATGCATAAAGCGGCTATTTCGCCGC
CTAAGAAAAAGATCGGGGGAAGTGAAAAATTTTCTAAAGTTCGAAATTCA
GGTGCCGATACAAGGGTTACGGTGAGAAACCGTGGGCAACAGCCCAATAA
CATCAAGTTGTAATTGATAAGGAAAAGATCATGGCACAAGTCATTAATAC
AAACAGCCTGTCGCTGTTGACCCAGAATAACCTGAACAAATCCCAGTCCG
CTCTGGGCACCGCTATCGAGCGTCTGTCTTCCGGTCTGCGTATCAACAGC
GCGAAAGACGATGCGGCAGGTCCGCTTGGTTTGGCTGGTCAGGTTCAACT
TGTTGAAAGCGGAGGCGGATTAGCTCAACCCGGCGGCTCTTTACGTCTTT
CCTGCGCTGCCTCTGGGTCCACTATTAGCTCCGTTGCCGTGGGTTGGTAC
CGTCAAACCCCAGGCAACCAACGTGAATGGGTTGCAACATCCAGCACCAG
TAGCACAACCGCCACCTATGCAGACTCGGTAAAGGGGCGCTTCACGATTA
GTCGTGACAACGCAAAAAACACCATCTACCTTCAAATGAACTCTCTGAAG
CCAGAGGACACAGCCGTGTACTATTGCAAGACGGGACTTACAAATTGGGG
GCGCGGAACGCAAGTTACCGTAAGCTCGGGTGGTGGCTACCCGTATGACG
TGCCTGACTACGCCTAAGCTTGACACG

*pFliC-αPD-L1N (N=nanobody)

To make pFliC-αPD-L1N, the pFliC-IL15 plasmid and the αPDL1N pFlic gBlock were digested with HindIII and DraIII. The digested plasmid was treated with alkaline phosphatase and ligated to the digested gBlock. The ligation was used to transform strain χ6212, isolate plasmid DNA, and the plasmids were sequenced to verify the FliC-αPDL1N construct. The correct plasmid was used to transform strain χ3730A; plasmid was isolated from the transformants and used to transform strain BCT13.

αPDL1N pFlic gBlock (730 bp):
CCAGTCCACAGTGTGCGGAATAATGATGCATAAAGCGGCTATTTCGCCGC
CTAAGAAAAAGATCGGGGGAAGTGAAAAATTTTCTAAAGTTCGAAATTCA
GGTGCCGATACAAGGGTTACGGTGAGAAACCGTGGGCAACAGCCCAATAA
CATCAAGTTGTAATTGATAAGGAAAAGATCATGGCACAAGTCATTAATAC
AAACAGCCTGTCGCTGTTGACCCAGAATAACCTGAACAAATCCCAGTCCG
CTCTGGGCACCGCTATCGAGCGTCTGTCTTCCGGTCTGCGTATCAACAGC
GCGAAAGACGATGCGGCAGGTCCATTAGGTTTAGCCGGCCAAGTCCAGCT
TGTCGAAACCGGAGGTGGACTGGTGCAGCCTGGAGGGTCACTGCGCCTTT
CCTGTACCGCATCTGGTTTCACTTTCAGTATGCACGCTATGACTTGGTAT
CGTCAGGCTCCTGGTAAGCAACGTGAACTTGTCGCGGTCATTACGAGCCA
TGGAGATCGCGCGAACTATACGGACTCAGTACGTGGTCGCTTTACAATCT
CCCGCGACAACACTAAGAATATGGTCTATTTGCAAATGAATAGCCTGAAG
CCTGAAGATACAGCGGTCTATTATTGTAACGTACCTCGTTACGACAGTTG
GGGCCAGGGGACTCAAGTAACGGTTTCATCTGGTGGAGGCTATCCCTATG
ATGTGCCAGATTACGCGTAAGCTTGACACG

*pFliC-αCD47N (N=nanobody)

To make pFliC-αCD47N, the pFliC-IL15 plasmid and the αCD47 pFliC gBlock were digested with HindIII and DraIII. The digested plasmid was treated with alkaline phosphatase and ligated to the digested gBlock. The ligation was used to transform strain χ6212, isolate plasmid DNA, and the plasmids were sequenced to verify the FliC-αCD47N construct. The correct plasmid was used to transform strain χ3730A; plasmid was isolated from the transformants and used to transform strain BCT13.

αCD47 pFliC gBlock (751 bp):
CCAGTCCACAGTGTGCGGAATAATGATGCATAAAGCGGCTATTTCGCCGC
CTAAGAAAAAGATCGGGGGAAGTGAAAAATTTTCTAAAGTTCGAAATTCA
GGTGCCGATACAAGGGTTACGGTGAGAAACCGTGGGCAACAGCCCAATAA
CATCAAGTTGTAATTGATAAGGAAAAGATCATGGCGCAGGTAATCAATAC
AAACTCCTTAAGTCTTTTAACTCAAAATAATCTTAACAAGAGTCAATCTG
CGCTTGGCACTGCTATCGAGCGGTTGTCTAGCGGGTTACGCATCAATTCT
GCCAAGGATGATGCGGCGGGTCCACTTGGCTTAGCGGGCCAAGTGCAGTT
GGTGGAATCTGGCGGCGGTTTGGTCGAGCCGGGTGGATCGTTACGGTTAT
CTTGTGCTGCATCGGGAATCATCTTCAAGATCAACGACATGGGGTGGTAC
CGGCAGGCACCAGGTAAACGTCGTGAATGGGTAGCAGCGTCGACTGGTGG
GGATGAAGCCATTTATCGCGATTCCGTTAAGGACCGTTTTACTATTAGTC
GCGACGCCAAGAATTCCGTTTTTTTACAGATGAATAGTTTGAAACCGGAG
GATACCGCCGTCTATTACTGTACTGCTGTAATCTCAACAGACCGGGATGG
TACAGAGTGGCGTCGCTATTGGGGCCAGGGGACGCAGGTTACAGTGAGTT
CTGGTGGTGGCTATCCGTACGATGTCCCGGATTACGCGTAAGCTTGACAC
G

Construction of plasmids that contain immunomodulatory gene combinations

1. pFliC-IL15-αPDL1N (N=Nanobody) Bicistronic Operon

To make the bicistronic plasmid, pFliC-IL15-αPDL1N, the IL15-αPDL1N gBlock and pFliC-IL15 plasmid (see above) were digested with DraIII and HindIII. The digested plasmid was treated with alkaline phosphatase and ligated to the digested gBlock. The ligation was used to transform strain χ6212, isolate plasmid DNA, and the plasmids were sequenced to verify the FliC-IL15-αPDL1N construct. The correct plasmid was used to transform strain χ3730A; plasmid was isolated from the transformants and used to transform strain BCT13.

IL15-αPDL1N gBlock (1555 bp):
CCAGTCCACAGTGTGCGGAATAATGATGCATAAAGCGGCTATTTCGCCGCCTAAGAAAAAGATCGGGGGAAGTGA
AAAATTTTCTAAAGTTCGAAATTCAGGTGCCGATACAAGGGTTACGGTGAGAAACCGTGGGCAACAGCCCAATAA
CATCAAGTTGTAATTGATAAGGAAAAGATCATGGCACAAGTCATTAATACAAACAGCCTGTCGCTGTTGACCCAG
AATAACCTGAACAAATCCCAGTCCGCTCTGGGCACCGCTATCGAGCGTCTGTCTTCCGGTCTGCGTATCAACAGC
GCGAAAGACGATGCGGCAGGTCCGTTAGGACTGGCTGGTACGACCTGTCCTCCCCCAGTTTCAATCGAGCACGCG
GATATCCGCGTTAAAAACTACTCCGTCAATAGTCGTGAACGTTATGTATGCAATTCCGGTTTCAAGCGTAAAGCT
GGGACATCTACCTTGATTGAGTGCGTAATCAACAAAAACACCAACGTGGCCCACTGGACGACGCCGTCCTTGAAG
TGCATTCGGGATCCCTCTTTAGCGCATTACAGTCCGGTGCCAACGTCAGGTGGTAGTGGCGGAGGCGGTTCAGGC
GGCGGGTCTGGAGGTGGTGGGAGCTTACAGAACTGGATCGATGTGCGGTATGATTTAGAGAAGATTGAATCCTTG
ATCCAATCTATTCACATTGACACAACCTTGTATACTGATTCAGATTTTCACCCCTCTTGCAAGGTTACGGCTATG
AATTGTTTTCTGCTGGAGTTACAAGTGATTCTTCACGAGTACTCTAATATGACTTTGAATGAAACAGTCCGCAAT
GTGTTGTATCTTGCTAACTCCACCCTGAGCAGTAACAAGAACGTTGCTGAAAGCGGTTGCAAAGAATGTGAGGAG
CTTGAGGAGAAGACCTTCACGGAGTTCCTGCAGTCATTCATTCGCATTGTTCAAATGTTTATTAACACTTCCTGA
TTGTTGGAATACGCCATAAACTGGGCGGGTATGGCGCAGGTCATTAACACTAATAGTCTGAGTCTGTTGACACAG
AACAACTTAAATAAGTCTCAGAGCGCTTTAGGCACCGCTATTGAACGTCTGAGTTCGGGGCTTCGCATCAATTCA
GCAAAAGATGACGCAGCGGGACCTTTAGGGTTGGCAGGTCAAGTCCAGCTTGTTGAAACCGGCGGCGGCTTAGTT
CAGCCCGGCGGTTCCCTGCGTTTAAGTTGCACTGCCTCGGGATTTACCTTCAGCATGCACGCAATGACTTGGTAT
CGGCAAGCTCCAGGAAAGCAGCGGGAATTAGTCGCAGTAATTACTAGCCATGGAGACCGGGCAAATTACACAGAC
TCCGTGCGCGGACGGTTCACAATTTCCCGCGACAATACTAAGAACATGGTTTATTTACAAATGAATTCATTAAAG
CCTGAGGACACCGCCGTCTACTACTGCAATGTCCCACGGTATGACAGTTGGGGGCAAGGCACCCAAGTAACGGTC
TCCTCGGGTGGCGGTTATCCTTACGATGTCCCCGATTATGCGTAAGCTTGACACC

2. pFliC-P Contains a Pentacistronic Operon with IL12, IL18, IL15, αPDL1N, and αCTLA4N (N=Nanobody)

To make the pFliC-P plasmid with a pentacistronic operon, two steps were used. The first step involved cloning the IL12 gene. The second step added the remaining four gene sequences to the pFliC-IL12 plasmid.

Step 1: The pFliC-IL12D gBlock and pFliC-IL15 plasmid (see above) were digested with DraIII and HindIII. The digested plasmid was treated with alkaline phosphatase and ligated to the digested gBlock. The ligation was used to transform strain χ6212, isolate plasmid DNA, and the plasmids were sequenced to verify the FliC-IL12D construct.

pFliC-IL12D gBlock (1960 bp):
CCAGTCCACAGTGTGCGGAATAATGATGCATAAAGCGGCTATTTCGCCGCCTAAGAAAAAGATCGGGGGAAGTGA
AAAATTTTCTAAAGTTCGAAATTCAGGTGCCGATACAAGGGTTACGGTGAGAAACCGTGGGCAACAGCCCAATAA
CATCAAGTTGTAATTGATAAGGAAAAGATCATGGCACAAGTCATTAATACAAACAGCCTGTCGCTGTTGACCCAG
AATAACCTGAACAAATCCCAGTCCGCTCTGGGCACCGCTATCGAGCGTCTGTCTTCCGGTCTGCGTATCAACAGC
GCGAAAGACGATGCGGCAGGTCCGCTCGGGCTGGCGGGGATGTGGGAACTCGAAAAGGATGTGTACGTTGTTGAA
GTTGACTGGACTCCCGACGCTCCAGGGGAAACAGTAAACTTAACTTGTGATACTCCCGAAGAGGACGATATCACG
TGGACTTCCGACCAACGGCACGGCGTAATCGGCTCCGGGAAGACCCTTACTATTACAGTTAAGGAATTCCTGGAC
GCTGGTCAGTATACTTGTCACAAAGGCGGTGAAACTCTGTCCCATAGCCATTTACTGCTGCACAAAAAGGAAAAC
GGCATTTGGAGCACTGAGATCCTTAAAAATTTCAAGAATAAAACCTTTCTGAAATGCGAAGCGCCTAACTATAGC
GGTCGGTTTACTTGTTCCTGGTTGGTACAGCGTAACATGGACTTGAAATTCAATATCAAGTCGTCTTCCTCGAGC
CCTGATTCGCGTGCAGTAACGTGCGGCATGGCGTCGTTATCCGCCGAGAAAGTCACATTAGATCAGCGGGACTAT
GAGAAGTATAGCGTTAGTTGTCAAGAGGACGTGACGTGCCCCACGGCTGAGGAAACGTTACCGATTGAATTAGCG
CTTGAAGCCCGCCAACAGAATAAATACGAAAACTATAGCACGTCTTTCTTTATCCGCGATATCATTAAACCTGAT
CCCCCGAAAAATTTACAGATGAAACCGTTGAAAAACTCACAGGTTGAGGTATCCTGGGAATATCCGGACTCTTGG
AGTACACCGCATTCGTACTTTTCGCTTAAGTTCTTTGTACGTATTCAACGGAAAAAGGAGAAGATGAAGGAGACC
GAGGAGGGATGCAATCAAAAGGGAGCTTTCTTAGTCGAGAAGACATCTACGGAAGTGCAGTGCAAGGGGGGCAAC
GTCTGTGTACAGGCACAGGATCGCTACTACAATTCGTCTTGCAGCAAGTGGGCGTGTGTTCCCTGCCGTGTACGG
AGTGGCGGAGGTGGCTCTGGCGGGGGGGGCAGCGGCGGGGGCGGCTCAAACCACCTTTCCTTAGCACGGGTGATC
CCGGTAAGCGGCCCAGCGCGCTGTCTGTCCCAAAGTCGGAATCTGTTAAAGACCACAGACGACATGGTCAAGACC
GCCCGTGAGAAGTTAAAGCACTATAGTTGTACAGCTGAAGACATTGACCACGAAGACATCACTCGCGACCAAACA
TCAACACTTAAAACCTGCTTACCGTTAGAACTTCATAAAAATGAGAGCTGCTTGGCGACCCGTGAAACGTCAAGC
ACTACTCGGGGAAGTTGCTTGCCCCCACAGAAGACTTCGTTAATGATGACCCTGTGCTTAGGTAGTATCTATGAG
GACCTTAAGATGTACCAAACTGAGTTCCAAGCAATCAACGCAGCGTTACAAAATCATAACCATCAACAAATCATT
CTTGATAAGGGGATGCTGGTGGCTATTGACGAACTTATGCAGTCACTTAATCATAATGGCGAAACCCTCCGACAA
AAGCCGCCGGTGGGAGAAGCGGATCCTTACCGTGTGAAGATGAAACTGTGCATTCTTCTTCATGCCTTTTCCACG
CGTGTGGTGACGATCAATCGGGTGATGGGATATTTGTCCAGTGCGGGTGGTGGCCATCATCACCATCACCATTAA
GCTTGACACG

Step 2: The “penta gBlock 2 M-H” gBlock and plasmid pFliC-IL12D were digested with MluI and HindIII. The digested plasmid was treated with alkaline phosphatase and ligated to the digested gBlock. The ligation was used to transform strain χ6212, isolate plasmid DNA, and the plasmids were sequenced to verify the FliC-P construct. The correct plasmid was used to transform strain χ3730A; plasmid was isolated from the transformants and used to transform strain BCT13.

“penta gBlock 2 M-H” gBlock (2663 bp):
GATAAGACGCGTGTGGTGACGATCAATCGGGTGATGGGATATTTGTCCAGTGCGTAATTTGCCAGTCTGAGGCGG
TAGATCTATGGCTCAAGTAATTAACACAAACTCGCTTTCCCTGTTGACGCAGAATAACCTGAATAAATCACAGTC
CGCATTGGGTACCGCCATTGAGCGGTTAAGCTCTGGTTTACGGATCAACTCTGCGAAAGATGATGCTGCGGGCCC
GCTCGGATTGGCGGGCCACTTTGGTCGTTTGCACTGTACGACTGCCGTTATACGGAACATCAATGACCAAGTCCT
ATTTGTGGACAAGCGCCAGCCAGTTTTTGAAGACATGACCGACATCGATCAGTCAGCGTCCGAACCTCAGACGCG
GCTTATTATCTATGCGTATGGTGACTCTCGAGCCCGCGGAAAAGCTGTGACGCTGAGTGTAAAGGACAGCAAGAT
GTCAACATTGTCATGTAAGAACAAGATCATCTCTTTCGAAGAAATGGACCCCCCTGAAAACATTGATGACATCCA
GAGCGATCTTATCTTCTTCCAGAAACGGGTGCCCGGTCATAATAAGATGGAATTTGAGTCGAGCCTGTATGAGGG
CCATTTCCTTGCTTGCCAAAAGGAGGACGATGCCTTCAAATTGATCCTGAAGAAAAAGGATGAAAACGGAGATAA
ATCAGTAATGTTTACCCTTACCAACTTACACCAGTCTTAACCCGTAAGGTAAGGAACTAACAAATATGGCACAGG
TCATTAACACGAATAGTCTGTCTCTGTTAACGCAAAATAACTTGAATAAGTCCCAGTCGGCTTTGGGGACCGCAA
TCGAGCGCTTGTCAAGTGGTCTTCGTATTAACTCTGCAAAAGACGATGCAGCAGGTCCGTTAGGACTGGCTGGTA
CGACCTGTCCCCCCCCAGTTTCAATCGAGCACGCGGATATCCGCGTTAAAAACTACTCCGTCAATAGTCGTGAAC
GTTATGTATGCAATTCCGGTTTCAAGCGTAAAGCTGGGACATCTACCTTGATTGAGTGCGTTATCAACAAAAACA
CCAACGTGGCCCACTGGACGACGCCGTCCTTGAAGTGCATTCGGGATCCCTCTTTAGCGCATTACAGTCCGGTGC
CAACGTCAGGGGGCAGTGGAGGCGGCGGTTCGGGTGGAGGCTCTGGAGGTGGTGGGAGCTTACAGAACTGGATAG
ATGTGCGGTATGATTTAGAGAAGATTGAATCCTTGATCCAATCTATTCACATTGACACAACCTTGTATACTGATT
CAGATTTTCACCCCTCTTGCAAGGTTACGGCTATGAATTGTTTTCTGCTGGAGTTACAAGTGATTCTTCACGAGT
ACTCTAACATGACTCTGAATGAAACGGTTCGCAACGTGTTGTATCTTGCTAACTCCACCCTGAGCAGTAACAAGA
ACGTTGCTGAGAGCGGTTGCAAAGAATGTGAGGAGCTTGAGGAGAAGACCTTCACGGAGTTCCTGCAGTCATTCA
TTCGCATCGTTCAAATGTTTATTAATACTTCATGATTGTAACGTTTAAGAAAGCCTCCCAATGGCGCAAGTGATT
AACACTAATAGTCTGAGTCTGTTGACACAGAACAACTTAAATAAGTCTCAGAGCGCTTTAGGCACCGCTATTGAA
CGTCTGAGTTCGGGGCTTCGCATCAATTCAGCAAAAGATGACGCAGCGGGACCTTTAGGGTTGGCAGGTCAGGTC
CAGCTTGTAGAAACAGGCGGCGGCTTAGTTCAGCCTGGCGGTTCCCTGCGTTTAAGTTGCACTGCCTCGGGATTT
ACCTTCAGCATGCACGCAATGACTTGGTATCGGCAAGCTCCAGGAAAGCAGCGGGAATTAGTCGCAGTAATTACT
TCGCATGGAGACCGGGCGAATTACACAGACTCCGTGCGCGGACGCTTCACAATTTCCCGCGACAATACCAAAAAC
ATGGTTTATCTGCAAATGAATTCCCTGAAACCAGAGGATACCGCTGTTTACTACTGCAATGTCCCACGGTATGAC
AGTTGGGGGCAGGGCACCCAAGTAACGGTCTCCTCGGGTGGCGGTTATCCTTACGATGTCCCCGATTATGCGTAA
ATCGTAAGATTAAGCCGAGACTCTCATGGCGCAGGTAATCAACACCAATTCCCTTAGCCTTCTGACGCAAAACAA
TCTTAACAAATCTCAGTCTGCCTTGGGCACGGCTATTGAGCGCCTGTCATCTGGCTTAAGAATCAATTCTGCGAA
GGATGATGCGGCGGGTCCGCTTGGTTTGGCTGGTCAGGTTCAACTTGTTGAAAGCGGAGGCGGATTAGCTCAACC
CGGCGGCTCTTTACGTCTTTCCTGCGCTGCCTCTGGGTCCACTATTAGCTCCGTTGCCGTGGGTTGGTACCGTCA
AACCCCAGGCAACCAACGTGAATGGGTTGCGACATCCAGCACCAGTAGCACAACCGCCACGTATGCAGACTCGGT
AAAGGGGCGCTTCACGATTAGTCGTGACAACGCAAAGAACACGATCTACCTTCAGATGAACTCTCTGAAGCCAGA
GGACACAGCCGTGTACTATTGTAAGACGGGACTTACAAATTGGGGGCGCGGAACGCAGGTTACCGTAAGCTCGGG
TGGTGGCCATCATCACCATCACCATTAAGCTTGACACC

3. pFliC-P-Lux contains a pentacistronic operon with IL12, IL18, IL15, αPDL1N, and
αCTLA4N and the lux operon (N=nanobody)

The lux operon was inserted into the pFliC-P plasmid in order to track tumor colonization by bioluminescence.

pGRG36-LacUV5-Lux was used as the source of the LacUV5-Lux operon.

The LacUV5-Lux operon was amplified from pLux (6) using the following primers:

Lux PacI Fwd 60.2:
5′ ACT GTT AAT TAA GAA GAC CTT CCA TTC TG 3′
Lux XhoI Rev 60.4:
5′-AGG ATT CTC GAG TTA TCA ACT ATC AAA CGC TTC
GGT TAA G-3′

The PCR product was digested with PacI and XhoI and ligated to pGRG36 (7) cleaved with the same enzymes. The recombinant pGRG36-LacUV5-Lux plasmid was confirmed by restriction digest of the plasmid and bioluminescence of the bacteria containing this plasmid. The pGRG36-LacUV5-Lux plasmid was cut with PacI and XhoI and the ends blunted. pFliC-P was cut with HindIII, ends blunted, and treated with alkaline phosphatase. These two cut plasmids were ligated and used to transform strain χ6212. The resulting colonies were screened for bioluminescence; plasmid was isolated from the positive clones, and the construct was verified by PCR and sequencing. The plasmid was used to transform strain χ3730A, and plasmid was isolated from the transformants. The plasmid was used to transform strain BCT14, and Lux expression was again verified by colony bioluminescence.

### BIBLIOGRAPHY

1. Galan J E, Nakayama K, Curtiss III R. 1990. Cloning and characterization of the asd gene of Salmonella typhimurium: use in stable maintenance of recombinant plasmids in Salmonella vaccine strains. Gene 94: 29-35.
2. Ryan R M, Green J, Williams P J, Tazzyman S, Hunt S, Harmey J H, Kehoe S C, Lewis C E. 2009. Bacterial delivery of a novel cytolysin to hypoxic areas of solid tumors. Gene Therapy 16:329-339.
3. Hess J, Gentschev I, Goebel W, Jarchau T. 1990. Analysis of the haemolysin secretion system by PhoA-HlyA fusion proteins. Mol Gen Genet. 224: 201-208.
4. Augustin L B, Milbauer L, Hastings S E, Leonard A S, Saltzman D A, Schottel J L. 2021. Virulence-attenuated Salmonella engineered to secrete immunomodulators reduce tumour growth and increase survival in an autochthonous mouse model of breast cancer. J. Drug Target. 29:430-438.
5. Green C A, Kamble N S, Court E K, Bryant O J, Hicks M G, Lennon C, Fraser G M, Wright P C, Stafford G P. 2019. Engineering the flagellar type III secretion system: improving capacity for secretion of recombinant protein. Microb Cell Fact 12:10-27.
6. Augustin L B, Milbauer L, Hastings S E, Leonard A S, Saltzman D A, Schottel J L. 2021. Salmonella enterica Typhimurium engineered for nontoxic systemic colonization of autochthonous tumors. J. Drug Target. 29:294-299. DOI: 10.1080/1061186X.2020.1818759 Published online 10 Sep. 2020.
7. McKenzie G J, Craig N L. 2006. Fast, easy and efficient: site-specific insertion of transgenes into Enterobacterial chromosomes using Tn7 without need for selection of the insertion event. BMC Microbiol. 6:39-45.

### Example IV

### Strain Construction

BCT2 (χ11091 plus fliC-fliB-fimH-rfaL-pgtE*)

Salmonella enterica Typhimurium strain χ11091 was used as the starting point for making strain BCT2, which had the following changes made to reduced toxicity: Delete the fliC gene; Delete the fljB gene; Delete the fimH gene; Delete the rfaL gene; and Single nucleotide change in the pgtE promoter to increase expression

All of these changes were made using the DIRex protocol (1). The details of these experiments were described previously (2).

BCT2E (BCT2 plus eca-)

The enterobacterial common antigen (3) locus (eca) was deleted in BCT2 to reduce toxicity using the DIRex protocol (1) and the following primers:

eca FP1
AAGTGTAATGTTCTAACAGGTCTCTTCTAGGAGCTGGCGTTCTCCCTTC
TGATGAGTGTAGGCTGGAGCTGCTTC
eca RP1
CCATAAGCACAATCCGCTGCTCAAGTCATCAGAAGGGAGAACGCCAGCT
CCTAGAGTGTAGGCTGGAGCTGCTTC

TABLE 1
Toxicity of strains BCT2 and BCT2E. Based on the data from two cohorts of nine mice each, strain BCT2E injected into the tail vein of non-tumor burdened Balb/C mice results in less than half the toxicity observed with strain BCT2. All mice received 100 microliter injections as described previously (2,11) and modified as follows. Day 2: (4 mg/kg VDA + 50 mg/kg CBD) IP. Day 0: 4 × 10⁶ cfu BCT2(pLux) or BCT2E(pLux) - 3 hr - 4 × 10⁶ cfu BCT2(pLux) or BCT2E(pLux) - 2 hr - (1 mg/kg VDA + 50 mg/kg CBD). Coat/mobility was observed on a scale of 0-3 = normal − poor. One mouse in the BCT2 cohort that developed extreme weight loss and became moribund and one mouse in the BCT2E cohort found dead on Day 1 were considered outliers and not included in the analysis.
BCT2pLux vs. BCT2EpLux Toxicity in Non-Tumor-Burdened Mice

| | Day 1 | | Day 2 | | Day 3 | | Day 4 | | Day 7 | |
|---|---|---|---|---|---|---|---|---|---|---|
| | | Coat/ | | Coat/ | | Coat/ | | Coat | | Coat/ |
| | % wt chg | mobility | % wt chg | mobility | % wt chg | mobility | % wt chg | mobility | % wt chg | mobility |
| | from Day 0 | (0-3) | from Day 0 | (0-3) | from Day 0 | (0-3) | from Day 0 | (0-3) | from Day 0 | (0-3) |
| BCT2 | −5.7 | 1/1 | −10.3 | 1/1 | −11.7 | 2/1 | −10.0 | 1/1 | −8.7 | 0/0 |
| | −5.2 | 2/1 | −10.0 | 2/1 | −11.4 | 2/ | −11.1 | 2/1 | −8.5 | 0/0 |
| | −10.3 | 2/1 | −13.9 | 2/1 | −15.7 | 2/1 | −13.5 | 2/1 | −4.5 | 0/0 |
| | −7.7 | 2/1 | −15.0 | 2/1 | −14.1 | 2/ | −13.2 | 2/1 | −9.4 | 1/0 |
| | −3.8 | 1/1 | −14.5 | 2/1 | −14.0 | 2/1 | −11.5 | 2/1 | −8.9 | 1/0 |
| | −3.6 | 1/1 | −9.9 | 2/1 | −10.3 | 2/— | −8.3 | 2/1 | −7.5 | 1/0 |
| | −4.9 | 1/1 | −9.0 | 1/1 | −12.6 | 2/ | −13.9 | 2/1 | −9.4 | 1/0 |
| | −3.8 | 1/1 | −6.4 | 2/1 | −10.6 | 2/— | −8.5 | 1/1 | −5.5 | 0/0 |
| | −7.4 | 2/1 | −13.7 | 2/1 | −16.0 | 2/1 | −14.8 | 2/1 | −13.3 | 1/0 |
| AVG | 5.8 | | −11.4 | | −12.9 | | −11.6 | | −8.4 | |
| BCT2E | −5.6 | 1/1 | −4.9 | 1/1 | −5.6 | 1/1 | −5.2 | 0/1 | −6.3 | 0/0 |
| | −7.9 | 2/1 | −10.4 | 2/1 | −10.4 | 2/— | −10.8 | 1/1 | −9.1 | 0/0 |
| | −7.3 | 2/1 | −6.1 | 1/1 | −3.1 | 0/ | −3.8 | 0/1 | −4.2 | 0/0 |
| | −9.2 | 2/1 | −10.9 | 2/1 | −9.2 | 2/1 | −8.7 | 0/1 | −1.3 | 0/0 |
| | −6.9 | 2/1 | −3.7 | 2/1 | −2.3 | 1/1 | −3.2 | 0/1 | −1.4 | 0/0 |
| | −7.3 | 1/1 | −5.3 | 1/1 | −3.7 | 1/1 | −4.9 | 0/1 | −4.1 | 0/0 |
| | −7.7 | 2/1 | −5.1 | 1/1 | −3.4 | 1/1 | −3.4 | 0/1 | −0.9 | 0/0 |
| | −6.5 | 1/1 | −4.6 | 1/1 | −4.6 | 1/1 | −4.6 | 0/1 | −3.5 | 0/0 |
| | −8.2 | 2/ | −6.3 | 1/1 | −6.0 | 1/1 | −7.8 | 0/1 | −7.1 | 0/0 |

| | | | | | |
|---|---|---|---|---|---|
| AVG | −7.4 | −6.4 | −5.3 | −5.8 | −4.2 |

BCT5 (BCT2 Plus rpoS-Plus viaB)

The rpoS gene codes for an RNA polymerase sigma factor (4). This gene was deleted using the DIRex protocol (1) and the following primers.

rpoS FP1
TTGCTAGTTCCGTCAAGGGATCACGGGTAGGAGCCACCTTGTACCCTTG
TCAAAAGTGTAGGCTGGAGCTGCTTC
rpoS RP1
AAGGCCAGTCGACAGACTGGCCTTTTTTTGACAAGGGTACAAGGTGGCT
CCTACCGTGTAGGCTGGAGCTGCTTC

The locus coding for the ViaB capsule was PCR-amplified from Salmonella Typhi Ty2 (5,6) and inserted into the BCT2 chromosome at the attTn7 site using lambda red recombination (7).

viaB PCR forward primer:
5′-CGGGGTACCAGTATGACGTTCTGACGGTT-3′
viaB PCR reverse primer:
5′-CGGGGTACCATTTTCAGCTCTGAAGTACA-3

BCT14 (BCT2 Plus fliA-rflP-flgKL-motAB-; flhDp→FF+20*; eca-)

Several deletions were made to the BCT2 strain to utilize the flagellar type III secretion system (8) for secretion of expressed immunomodulators from the bacteria into the tumor microenvironment and to further reduce toxicity. All the changes were made using the DIRex protocol (1) and the indicated primers.

Delete the fljA gene
fljA FP1
ATTCGGGGCTTTTTCATTTAGCATAGATGAATATATATTTCGCCTACGG
TAATAAGTGTAGGCTGGAGCTGCTTC
fljA RP1
AGTTTTACTTTTCTCACGGAATTTTTTATTACCGTAGGCGAAATATATA
TTCATCGTGTAGGCTGGAGCTGCTTC
Delete the rflP gene
rflP FP1
ACTGGATGGCGAATAGCGCCCTAACCATGGGACTGGCGTACCTTTCTGT
TTGCCGGTGTAGGCTGGAGCTGCTTC
rflP RP1
AGACGGTTAATCACCGGTTAAACACCGGCAAACAGAAAGGTACGCCAGT
CCCATGGTGTAGGCTGGAGCTGCTTC
Delete the flgKL genes
flgKL FP1
GCCGATAACAACGAGTATTGAAGGATTAAAAGGAACCATCCACCTCTTT
TTGAAAGTGTAGGCTGGAGCTGCTTC
flgKL RP1
AAAACATATCCAGTTTCGTGATATGTTTCAAAAAGAGGTGGATGGTTCC
TTTTAAGTGTAGGCTGGAGCTGCTTC
Delete the motAB genes
motAB FP1
CTGCGCATCCTGTCATAGTCAACAGCGGAAGGATGATGTCTAGCGTGAG
CATGGAGTGTAGGCTGGAGCTGCTTC
motAB RP1
AAATGTCTGATAAAAATCGCTAATATCCATGCTCACGCTAGACATCATC
CTTCCGGTGTAGGCTGGAGCTGCTTC

The flhDp promoter sequence for expression of the flagellar genes was replaced with the tumor-specific FF+20* promoter (9) to confine expression of these genes to the tumor microenvironment.

Promoter replacement: flhDp→FF+20*

flhDp → FF + 20* FP1-2
GGATGCTTCATTTAAATGGGTGAACAAGGAAAGCTAAAAGGGATCAGGT
AAATTTGATGTACATCAAATGGATCCAGATCGATGTTTAAAGATCCCCC
CTCACTCCTGCCATCATTCTGATATTGAAATAAGAGAGGAGGAACATAA
GTGTAGGCTGGAGCTGCTTC
flhDp → FF + 20* RP1
AAATGTGTTTTAGCAACTCGGATGTATGCATTGTTCCCATTTATGTTCC
TCCTCTCTTATTTCAATATCAGAATGATGGCAGGAGTGAGGGGGGATCT
TTAAACATCGATCTGGATCCATTTGATGTACATCAAATTTACCTGATCC
GTGTAGGCTGGAGCTGCTTC

Delete the eca locus (3) to decrease toxicity as for the BCT2E strain

eca FP1
AAGTGTAATGTTCTAACAGGTCTCTTCTAGGAGCTGGCGTTCTCCCTTC
TGATGAGTGTAGGCTGGAGCTGCTTC
eca RP1
CCATAAGCACAATCCGCTGCTCAAGTCATCAGAAGGGAGAACGCCAGCT
CCTAGAGTGTAGGCTGGAGCTGCTTC

### BCT14-PL-Lux

The PL-Lux operon was inserted into the chromosome at the attTn7 site using lambda red recombination (7). In this construct, the lux operon is driven by the lambda PL promoter (10). The lux operon was amplified from pLux (2) using the following primers, which contain the PL promoter sequence and relevant restriction enzyme sites:

PL-Lux PacI Fwd:
5′-ACT GTT AAT TAA TTA TCT CTG GCG GTG TTG ACA
TAA ATA CCA CTG GCG GTG ATA CTG AGC ACA TCA GCA
GGA CGC ACT GAC CCA CAA TAG GAG GAA CAT AAA TGA
CTA AA-3′
Lux XhoI Rev 60.4:
5′-AGG ATT CTC GAG TTA TCA ACT ATC AAA CGC TTC
GGT TAA G-3′

The PCR product was digested with PacI and XhoI and ligated to pGRG36 (7) cleaved with the same enzymes. The recombinant pGRG36-PL-Lux plasmid was confirmed by restriction digest of the plasmid and bioluminescence of the bacteria containing this plasmid. The PL-Lux operon from pGRG36-PL-Lux was then inserted into the BCT13 chromosome at the attTn7 site using lambda red recombination (7) followed by deletion of the eca locus (3) as described for BCT2E.

### BIBLIOGRAPHY

1. Nasvall J. Direct and inverted repeat stimulated excision (DIRex): simple, single-step, and scar-free mutagenesis of bacterial genes. PLoS One. 2017; 12(8):e0184126.
2. Augustin L B, Milbauer L, Hastings S E, Leonard A S, Saltzman D A, Schottel J L. 2021. Salmonella enterica Typhimurium engineered for nontoxic systemic colonization of autochthonous tumors. J. Drug Target. 29:294-299. DOI: 10.1080/1061186X.2020.1818759 Published online 10 Sep. 2020.
3. Gilbreath J. J., Dodds J C, Rick P D, Solosk, M J, Merrell D S, Metcalf E S. 2012. Enterobacterial common antigen mutants of Salmonella enterica serovar Typhimurium establish a persistent infection and provide protection against subsequent lethal challenge. Infect. Immun. 80:441-450.
4. Santander J, Wanda S-Y, Nickerson C A, Curtiss III R. 2007. Role of RpoS in fine-tuning the synthesis of Vi capsular polysaccharide in Salmonella enterica serotype Typhi. Infect. Immun. 75:1382-1392.
5. Haneda T, Winter S E, Butler B P, Wilson R P, Tukel C, Winter M G, Godinez I, Tsolis R M, Baumler A J. 2009. The capsule-encoding viaB locus reduces intestinal inflammation by a Salmonella pathogenicity island 1-independent mechanism. Infect. Immun. 77:2932-2942.
6. Xiong K, Zhu C, Chen Z, Zheng C, Tan Y, Rao X, Cong Y. 2017. Vi capsular polysaccharide produced by recombinant Salmonella enterica serovar Paratyphi A confers immunoprotection against infection by Salmonella enterica serovar Typhi. Front. Cell. Infect. Microbiol. 7:1-10.
7. McKenzie G J, Craig N L. 2006. Fast, easy and efficient: site-specific insertion of transgenes into Enterobacterial chromosomes using Tn7 without need for selection of the insertion event. BMC Microbiol. 6:39-45.
8. Green C A, Kamble N S, Court E K, Bryant O J, Hicks M G, Lennon C, Fraser G M, Wright P C, Stafford G P. 2019. Engineering the flagellar type III secretion system: improving capacity for secretion of recombinant protein. Microb Cell Fact 12:10-27.
9. Ryan R M, Green J, Williams P J, Tazzyman S, Hunt S, Harmey J H, Kehoe S C, Lewis C E. 2009. Bacterial delivery of a novel cytolysin to hypoxic areas of solid tumors. Gene Therapy 16:329-339.
10. Cheng X, Patterson T A. 1992. Construction and use of 1 P_{L} promoter vectors for direct cloning and high-level expression of PCR amplified DNA coding sequences. Nucl. Acid Res. 20:4591-4598.
11. Augustin L B, Milbauer L, Hastings S E, Leonard A S, Saltzman D A, Schottel J L. 2021. Virulence-attenuated Salmonella engineered to secrete immunomodulators reduce tumour growth and increase survival in an autochthonous mouse model of breast cancer. J. Drug Target. 29:430-438. Published online 21 Dec. 2020.

### Example V

### Strains:

BCT15 (BCT14 plus viaB locus)
   The locus coding for the ViaB capsule was PCR-amplified from Salmonella Typhi Ty2 (χ8073) (1,2) and inserted into the BCT14 chromosome at the attTn7 site using lambda red recombination (3).

viaB PCR forward primer:
5′-CGGGGTACCAGTATGACGTTCTGACGGTT-3′
viaB PCR reverse primer:
5′-CGGGGTACCATTTTCAGCTCTGAAGTACA-3′

BCT16 (BCT14 plus glmS-)

The glmS gene (4) was deleted in strains χ6212, χ3730A, and BCT14 to provide another balanced lethal selection in addition to the asd deletion. The DIRex protocol (5) was used with the following primers for the deletion in BCT14:

glmS FP1
GACAACGCATGGCGTAATGCCATAATCAGGAAATAAAACTGTACAAATG
TAGTACGTGTAGGCTGGAGCTGCTTC
glmS RP1
CCGTCTGTCGACGGCCTTCTGCCTGGTACTACATTTGTACAGTTTTATT
TCCTGAGTGTAGGCTGGAGCTGCTTC

### Plasmids:

pGlmS

pGlmS contains the glmS gene to complement the glmS chromosomal deletion in BCT16. To construct pGlmS, the pNG.1 plasmid was digested with BspH1 and BspE1 and ligated to pGlmS gBlock1 that had been cut with BspH1 and EcoR1-HF and to pGlmS gBlock2 that had been cut with EcoR1-HF and BspE1. The ligation was used to transform strain χ6212glmS-, isolate plasmid DNA, and the plasmids were sequenced to verify the pGlmS construct. The correct plasmid was used to transform strain χ3730A glmS- and plasmid was isolated from the transformants.

pGlmS gBlock1 (1058 bp)
5′-GCA ACG TCA TGA GAC AAT AAC CCT GAT AAA TGC TTC AAT AAT ATT GAA AAA GGA AGA GTA TGT GTG GAA
TTG TTG GCG CTA TCG CGC AGC GTG ATG TAG CTG AAA TCC TTC TCG AAG GTT TAC GTC GTC TGG AAT ACC GCG
GAT ATG ACT CTG CCG GTC TGG CCG TGG TGG ATG CAG AAG GTC ATA TGA CCC GCC TGC GTC GCC TCG GTA AAG
TCC AGA TGC TGG CTC AGG CAG CGG AAG AAC ATC CTC TGC ATG GCG GCA CTG GTA TTG CTC ACA CTC GCT GGG
CGA CGC ACG GTG AAC CTT CAG AAG CTA ACG CGC ATC CGC ATG TTT CTG AAC ACA TTG TGG TGG TGC ATA ACG
GCA TCA TCG AAA ACC ATG AAC CGC TGC GTG AAG CGT TAA AAG CGC GTG GCT ATA CCT TCG TTT CTG AAA CCG
ACA CCG AAG TGA TTG CAC ACC TGG TAA ACT GGG AAC TGA AAC AGG GCG GTA CTC TGC GTG AGG CTG TTC TGC
GTG CTA TCC CGC AAC TGC GTG GCG CAT ATG GTA CGG TAA TTA TGG ATA CCC GTC ACC CGG ATA CCC TAC TGG
CGG CAC GTT CTG GTA GCC CGC TGG TGA TTG GCC TGG GTA TGG GCG AAA ACT TTA TCG CTT CTG ACC AGC TGG
CGC TGT TGC CAG TGA CTC GTC GCT TTA TCT TCC TTG AAG AGG GCG ATA TCG CTG AAA TCA CTC GCC GTT CGG
TAA ATA TCT TCG ATA ACA CCG GCG CGG AAG TAA AAC GTC AGG ATA TCG AAT CCA ATC TGC AAT ATG ACG CGG
GCG ATA AAG GTA TTT ACC GCC ACT ACA TGC AGA AAG AGA TCT ACG AAC AGC CGA ACG CGA TCA AAA ACA CCC
TTA CCG GGC GCA TCA GCC ACG GTC AGG TGG ATT TAA GCG AGC TGG GAC CGA ACG CCG ACG AAC TGC TGT CGA
AGG TTG AGC ATA TTC AGA TCC TCG CCT GCG GTA CGT CTT ACA ACT CCG GTA TGG TTT CCC GTT ACT GGT TTG
AAT CGC TGG CAG GTA TTC CGT GCG ACG TTG AAA TCG CCT CTG AAT TCG GTA GT-3′
pGlmS gBlock2 (2404 bp)
5′-CCT ACC GAA TTC CGT TAT CGC AAA TCT GCC GTG CGC CGT AAC AGC CTG ATG ATC ACC CTG TCA CAG TCT
GGC GAA ACC GCA GAT ACC CTG GCT GGC CTG CGT CTG TCG AAA GAG CTG GGA TAT TTG GGC TCT CTG GCA ATT
TGT AAC GTG CCG GGC TCT TCG CTG GTG CGT GAA TCC GAT CTG GCG CTG ATG ACC AAC GCG GGT ACG GAA ATT
GGC GTA GCA TCT ACC AAA GCC TTC ACC ACC CAG TTA ACC GTG CTG TTG ATG CTG GTG GCG AAA CTG TCT CGC
CTG AAA GGT CTG GAT GCG TCC ATT GAG CAT GAT ATC GTT CAT GGC CTA CAG GCG CTG CCG AGC CGT ATT GAG
CAG ATG CTG TCG CAG GAC AAA CGC ATC GAG CTG CTT GCA GAA GAT TTC TCC GAC AAG CAT CAC GCG CTA TTC
CTG GGG CGT GGC GAT CAA TAT CCG ATT GCG CTC GAA GGC GCG CTG AAG CTG AAA GAG ATC TCT TAC ATT CAC
GCG GAA GCC TAT GCT GCA GGC GAG CTG AAA CAC GGG CCG TTG GCG CTG ATT GAC GCC GAT ATG CCG GTG ATT
GTG GTG GCG CCG AAC AAC GAA CTG CTG GAG AAA CTG AAA TCC AAC ATC GAA GAA GTT CGC GCA CGT GGC GGT
CAG TTG CAC GTC TTC GCC GAT CAG GAT GCG GGT TTC GTC AGC AAC GAC AAC ATG CAC ATC ATC GAG ATG CCG
CAT GTG GAA GAG GTC ATT GCG CCT ATC TTC TAC ACC GTT CCG CTG CAA CTG CTG GCG TAT CAC GTG GCG CTG
ATT AAA GGC ACC GAC GTT GAC CAG CCG CGT AAC CTG GCG AAA TCG GTT ACC GTA GAG TAA CTG TCA GAC CAA
GTT TAC TCA TAT ATA CTT TAG ATT GAT TTA AAA CTT CAT TTT TAA TTT AAA AGG ATC TAG GTG AAG ATC CTT
TTT GAT AAT CTG ATG ACC AAA ATC CCT TAA CGT GAG TTT TCG TTC CAC TGA GCG TCA GAC CCC GTA GAA AAG
ATC AAA GGA TCT TCT TGA GAT CCT TTT TTT CTG CGC GTA ATC TGC TGC TTG CAA ACA AAA AAA CCA CCG CTA
CCA GCG GTG GTT TGT TTG CCG GAT CAA GAG CTA CCA ACT CTT TTT CCG AAG GTA ACT GGC TTC AGC AGA GCG
CAG ATA CCA AAT ACT GTC CTT CTA GTG TAG CCG TAG TTA GGC CAC CAC TTC...-3′

pGlmS-CXCL9-10

To make pGlmS-CXCL9-10, the pGlmS plasmid was digested with NheI, blunted, and treated with alkaline phosphatase. pFliC-CXCL9-10 was digested with HindIII and DraIII, blunted and ligated to the digested pGlmS plasmid. The ligation was used to transform strain χ6212glmS-, isolate plasmid DNA, and the plasmids constructs were verified by sequencing. The correct plasmid was used to transform strain χ3730AglmS; plasmid was isolated from the transformants and used to transform strain BCT16.

pGlmS-αCD47N (N=Nanobody)

To make pGlmS-αCD47, the pGlmS plasmid was digested with NheI, blunted, and treated with alkaline phosphatase. pFliC-αCD47 was digested with HindIII and DraIII, blunted and ligated to the digested pGlmS plasmid. The ligation was used to transform strain χ6212glmS-, isolate plasmid DNA, and the plasmids constructs were verified by sequencing. The correct plasmid was used to transform strain χ3730AglmS-; plasmid was isolated from the transformants and used to transform strain BCT16.

pGlmS-INF (Interferon Alpha+Lambda Biscistronic Operon)

To make pGlmS-INF, the pGlmS plasmid and pGmsS-INF gBlock were digested with NheI and BsaI. The digested pGlmS plasmid was treated with alkaline phosphatase and ligated to the digested gBlock. The ligation was used to transform strain χ6212glmS-, isolate plasmid DNA, and the plasmids constructs were verified by sequencing. The correct plasmid was used to transform strain χ3730AglmS-; plasmid was isolated from the transformants and used to transform strain BCT16.

pGlmS-INF gBlock (1710 bp)
5′-AAG GTG GCT AGC GTG CGG AAT AAT GAT GCA TAA AGC GGC TAT TTC GCC GCC TAA GAA AAA GAT CGG GGG
AAG TGA AAA ATT TTC TAA AGT TCG AAA TTC AGG TGC CGA TAC AAG GGT TAC GGT GAG AAA CCG TGG GCA ACA
GCC CAA TAA CAT CAA GTT GTA ATT GAT AAG GAA AAG ATC ATG GCA CAA GTC ATT AAT ACA AAC AGC CTG TCG
CTG TTG ACC CAG AAT AAC CTG AAC AAA TCC CAG TCC GCT CTG GGC ACC GCT ATC GAG CGT CTG TCT TCC GGT
CTG CGT ATC AAC AGC GCG AAA GAC GAT GCG GCA GGT CCC TTA GGT TTA GCG GGG TGT GAC TTA CCG CAC ACG
TAC AAC TTG CGG AAC AAG CGG GCG TTG AAA GTG CTG GCT CAG ATG CGG CGC TTA CCA TTC TTA TCC TGC TTG
AAA GAT CGG CAA GAC TTC GGG TTC CCA TTA GAG AAG GTG GAT AAC CAA CAA ATC CAG AAA GCG CAA GCT ATC
CCA GTT CTT CGA GAC TTG ACC CAG CAA ACG CTG AAT TTG TTT ACT TCT AAA GCC TCC TCC GCG GCA TGG AAT
ACG ACT TTG TTG GAT TCT TTC TGC AAC GAC CTC CAC CAA CAG TTG AAC GAT TTA CAG ACG TGC CTG ATG CAG
CAA GTT GGA GTC CAA GAA CCA CCC TTA ACA CAG GAA GAC GCG CTG TTA GCT GTC CGT AAA TAC TTT CAC CGT
ATC ACT GTC TAT TTA CGG GAA AAG AAA CAT TCA CCG TGT GCT TGG GAA GTC GTG CGC GCC GAG GTT TGG CGT
GCC CTT TCT TCT TCC GTG AAC TTG TTG CCT CGG TTG TCA GAA GAG AAA GAA TAA GAA ACG CGC CTA AGG AGG
TCC TCA CAT GGC GCA GGT TAT CAA CAC GAA CTC CTT AAG CCT GTT GAC TCA GAA CAA CTT GAA CAA AAG CCA
ATC AGC ACT CGG AAC AGC GAT AGA ACG CTT ATC GTC GGG ATT GCG GAT CAA TTC TGC CAA AGA CGA TGC GGC
CGG ACC CTT GGG TCT GGC TGG GGA TCC CGT GCC ACG TGC CAC ACG TCT GCC GGT TGA AGC CAA AGA CTG CCA
TAT TGC TCA GTT TAA GTC CTT ATC ACC TAA GGA ATT GCA AGC GTT TAA GAA GGC TAA GGA CGC GAT AGA GAA
ACG ATT GTT AGA AAA GGA TCT TCG GTG TAG CTC TCA TTT GTT TCC TCG TGC...-3′

### BIBLIOGRAPHY

1. Haneda T, Winter S E, Butler B P, Wilson R P, Tukel C, Winter M G, Godinez I, Tsolis R M, Baumler A J. 2009. The capsule-encoding viaB locus reduces intestinal inflammation by a Salmonella pathogenicity island 1-independent mechanism. Infect. Immun. 77:2932-2942.
2. Xiong K, Zhu C, Chen Z, Zheng C, Tan Y, Rao X, Cong Y. 2017. Vi capsular polysaccharide produced by recombinant Salmonella enterica serovar Paratyphi A confers immunoprotection against infection by Salmonella enterica serovar Typhi. Front. Cell. Infect. Microbiol. 7:1-10.
3. McKenzie G J, Craig N L. 2006. Fast, easy and efficient: site-specific insertion of transgenes into Enterobacterial chromosomes using Tn7 without need for selection of the insertion event. BMC Microbiol. 6:39-45.
4. Kim, K, Jeong J H, Lim D, Hong Y, Yun M, Min J-J, Kwak S-J, Choy H E. 2013. A novel balanced-lethal host-vector system based on glmS. PLOS One. 8:1-11.
5. Nasvall J. Direct and inverted repeat stimulated excision (DIRex): simple, single-step, and scar-free mutagenesis of bacterial genes. PLoS One. 2017; 12(8):e0184126.

### SEVERAL EMBODIMENTS

1. An attenuated Salmonella cell comprising:
a) a mutation or deletion in one or more of the Salmonella genes coding for cell surface proteins that induce IL-6 secretion, so as to result in reduced or no expression of said one or more cell surface proteins; and optionally
b) an increase in expression, as compared to a control cell, of one or more outer membrane proteases that inhibit complement activation and/or a decrease in expression of cell surface lipopolysaccharide protein (LPS).
   2. The cell of embodiment 1, where the cell is a S. Typhimurium cell.
   3. The cell of embodiment 1 or 2, wherein the one or more Salmonella genes code for flagellin, fimbriae, O-antigen and/or lipopolysaccharide protein (LPS).
   4. The cell of any one of embodiments 1 to 3, wherein the one or more Salmonella genes are fliC, fljB, fimH and/or rfaL.
   5. The cell of any one of embodiments 1 to 4, wherein the one or more outer membrane proteases is PgtE.
   6. The cell of any one of embodiment 1 to 5, further comprising a deletion of the enterobacterial common antigen locus (eca).
   7. The cell of any one of embodiments 1 to 6, further comprising a deletion of the rpoS gene, deletion of gmlS and/or the addition of a viaB locus.
   8. The cell of any one of embodiments 1 to 7, further comprising a deletion of one or more of fljA, rflP, flgKL and/or motAB genes.
   9. The cell of any one of embodiments 1 to 8, wherein the flhDP promoter was replaced with a tumor-specific expression promoter.
   10. The cell of embodiment 9, wherein the tumor-specific expression promoter is FF+20* promoter.
   11. The cell of any one of embodiments 1 to 10, wherein the cell comprises one or more exogenous immunomodulator genes which express an exogenous immunomodulator protein.
   12. The cell of embodiment 11, wherein the immunomodulator genes express IL-12, IL-18, IL-15, CXCL9-10, αCTLA-4 single-chain fragment variable (scFv), αPD-L1 scFv, αCTLA-4 single-domain antibody (sdAb), αPD-L1 sdAb and/or αCD47 sdAb.
   13. The cell of any one of embodiments 11 to 12, wherein the immunomodulator protein is secreted from the cell.
   14. The cell of any one of embodiments 11 to 13, wherein the one or more exogenous immunomodulator genes are under the control of a tumor-specific expression promoter.
   15. The cell of embodiment 14, wherein the tumor-specific expression promoter is FF+20*.
   16. A composition comprising a population of cells of any one of embodiments 1 to 15 or a combination thereof and a pharmaceutically acceptable carrier.
   17. A method to treat cancer comprising administering to subject in need thereof an effective amount of a population of the cells of any one of embodiments 1 to 15, a combination thereof or the composition of embodiment 16 so as to treat said cancer.
   18. A method of inhibiting tumor growth/proliferation or reducing the volume/size of a tumor comprising administering to subject in need thereof an effective amount of a population of the cells of any one of embodiments 1 to 15, a combination thereof or the composition of embodiment 16 so as to suppress tumor growth or reduce the volume of the tumor.
   19. A method to treat, reduce formation/number or inhibit spread of metastases comprising administering to subject in need thereof an effective amount of a population of the cells of any one of embodiments 1 to 15, a combination thereof or the composition of embodiment 16, so as to treat, reduce formation/number or inhibit spread of metastases.
   20. The method of any one of embodiments 17 to 19, wherein the tumor, cancer, or metastases are a lung, liver, kidney, breast, prostate, pancreatic, colon, head and neck, ovarian and/or gastroenterological tumor, tumor associated cells, cancer or metastases.
   21. The method of any one of embodiments 17 to 20, wherein the cells or composition is administered systemically, such as intravenously.
   22. The method of any one of embodiments 17 to 21, wherein the cells are administered more than once.
   23. The method of any one of embodiment 17 to 22, further comprising administering a vascular disrupting agent (VDA) and/or cannabidiol (CBD).
   24. The method of embodiment 24, wherein the VDA and/or CBD are administered prior to and/or during treatment (after at least one administration of the cells).
   25. The method embodiments 23 or 24, wherein the VDA and/or CBD are administered more than once.
   26. The method of any one of embodiments 23 to 25, wherein the VDA is VDA combretastatin A4 phosphate and or VDA CKD-516.
   27. The method of any one of embodiments 17 to 26, wherein anti-interleukin-6 (IL-6) is administered.
   28. A method to reduce toxicity of Salmonella comprising
a) deleting one or more of the Salmonella genes coding for cell surface proteins that induce IL-6 secretion, so as to result in reduced or no expression of said one or more cell surface proteins; and optionally
b) increasing expression, as compared to a control cell, of one or more outer membrane proteases that inhibit complement activation and/or decreasing expression of cell surface lipopolysaccharide protein (LPS).
   29. The method of embodiment 28, where the Salmonella is a S. Typhimurium cell.
   30. The method of embodiment 28 or 29, wherein the one or more Salmonella genes code for flagellin, fimbriae, O-antigen and/or lipopolysaccharide protein (LPS).
   31. The method of any one of embodiments 28 to 30, wherein the one or more Salmonella genes are fliC, fljB, fimH and/or rfaL.
   32. The method of any one of embodiments 28 to 31, wherein the one or more outer membrane proteases is PgtE.
   33. The method of any one of embodiments 28 to 32, further comprising a deletion of the enterobacterial common antigen locus (eca).
   34. The method of any one of embodiments 28 to 33, further comprising a deletion of the rpoS gene and/or the addition of a viaB locus.
   35. The method of any one of embodiments 28 to 34, further comprising a deletion of one or more of fljA, rflP, flgKL and/or motAB genes.
   36. The method of any one of embodiments 28 to 35, wherein the flhDP promoter is replaced with a tumor-specific expression promoter.
   37. The method of claim 36, wherein the tumor-specific expression promoter is FF+20* promoter.

All publications, patents, and patent applications mentioned in this specification are herein incorporated by reference to the same extent as if each individual publication, patent, or patent application was specifically and individually indicated to be incorporated by reference. In the event that the definition of a term incorporated by reference conflicts with a term defined herein, this specification shall control.

1. An attenuated Salmonella cell comprising:
a) a mutation or deletion in one or more of the Salmonella genes coding for cell surface proteins that induce IL-6 secretion, so as to result in reduced or no expression of said one or more cell surface proteins; and optionally
b) an increase in expression, as compared to a control cell, of one or more outer membrane proteases that inhibit complement activation and/or a decrease in expression of cell surface lipopolysaccharide protein (LPS).

2. The cell of claim 1, where the cell is a S. Typhimurium cell.

3. The cell of claim 1, wherein the one or more Salmonella genes code for flagellin, fimbriae, O-antigen and/or lipopolysaccharide protein (LPS).

4. The cell of claim 1, wherein the one or more Salmonella genes are fliC, fljB, fimH and/or rfaL.

5. The cell of claim 1, wherein the one or more outer membrane proteases is PgtE.

6. The cell of claim 1, further comprising a deletion of the enterobacterial common antigen locus (eca).

7. The cell of claim 1, further comprising a deletion of the rpoS gene, deletion of glmS and/or the addition of a viaB locus.

8. The cell of claim 1, further comprising a deletion of one or more of fljA, rflP, flgKL and/or motAB genes.

9. The cell of claim 1, wherein the flhDP promoter was replaced with a tumor-specific expression promoter.

10. (canceled)

11. The cell of claim 1, wherein the cell comprises one or more exogenous immunomodulator genes which express an exogenous immunomodulator protein.

12. The cell of claim 11, wherein the immunomodulator genes express IL-12, IL-18, IL-15, CXCL9-10, αCTLA-4 single-chain fragment variable (scFv), αPD-L1 scFv, αCTLA-4 single-domain antibody (sdAb), αPD-L1 sdAb and/or αCD47 sdAb.

13. (canceled)

14. The cell of claim 11, wherein the one or more exogenous immunomodulator genes are under the control of a tumor-specific expression promoter.

15. (canceled)

16. A composition comprising a population of cells of claim 1 or a combination thereof and a pharmaceutically acceptable carrier.

17. A method to treat cancer comprising administering to subject in need thereof an effective amount of a population of the cells of claim 1 so as to treat said cancer.

18. A method of inhibiting tumor growth/proliferation or reducing the volume/size of a tumor comprising administering to subject in need thereof an effective amount of a population of the cells of claim 1 so as to suppress tumor growth or reduce the volume of the tumor.

19. A method to treat, reduce formation/number or inhibit spread of metastases comprising administering to subject in need thereof an effective amount of a population of the cells of claim 1 so as to treat, reduce formation/number or inhibit spread of metastases.

20. The method of claim 17, wherein the tumor, cancer, or metastases are a lung, liver, kidney, breast, prostate, pancreatic, colon, head and neck, ovarian and/or gastroenterological tumor, tumor associated cells, cancer or metastases.

21-22. (canceled)

23. The method of claim 17, further comprising administering a vascular disrupting agent (VDA) and/or cannabidiol (CBD).

24-25. (canceled)

26. The method of claim 23 wherein the VDA is VDA combretastatin A4 phosphate and or VDA CKD-516.

27. The method of claim 17, wherein anti-interleukin-6 (IL-6) is administered.

28-37. (canceled)


SEQUENCE LISTING (77 sequences)

SEQ ID NO: 1 - protein, 18 aa, synthetic construct
GQSSRSSGGGGSSGGGGS

SEQ ID NO: 2 - DNA, 25 bp, synthetic construct
  source 1..25: mol_type = other DNA
ttagcctatggaagtcagggtaatc

SEQ ID NO: 3 - DNA, 27 bp, synthetic construct
  source 1..27: mol_type = other DNA
ttaacgctcatgtaaactttctgttac

SEQ ID NO: 4 - DNA, 101 bp, synthetic construct
  source 1..101: mol_type = other DNA
agatcttccggaagaccttccattctgaaatgagctgtttacactttatgcttccggctcgtataatgtgtggaattgtgagcggataacaatttcacaat

SEQ ID NO: 5 - DNA, 100 bp, synthetic construct
  source 1..100: mol_type = other DNA
agatcttccggaagaccttccattctgaaatgagctgttgacaattaatcatccggctcgtataatgtgtggaattgtgagcggataacaatttcacaat

SEQ ID NO: 6 - DNA, 1126 bp, synthetic construct
  source 1..1126: mol_type = other DNA
gatccttgtacatcaaatggatccagatcgatgtttaaagatcccccctcactcctgccatcattctgatattgaaataagagaggaggaacataaatgaagtccgctgttcttttcctcttgggcatcatcttcctggagcagtgtggagttcgaggaaccctagtgataaggaatgcacgatgctcctgcatcagcaccagccgaggcacgatccactacaaatccctcaaagacctcaaacagtttgccccaagccccaattgcaacaaaactgaaatcattgctacactgaagaacggagatcaaacctgcctagatccggactcggcaaatgtgaagaagctgatgaaagaatgggaaaagaagatcagccaaaagaaaaagcaaaagagggggaaaaaacatcaaaagaacatgaaaaacagaaaacccaaaacaccccaaagtcgtcgtcgttcaaggaagactacagttcccggggtaggagttccaggtgttggtgggatccctctcgcaaggacggtccgctgcaactgcatccatatcgatgacgggccagtgagaatgagggccatagggaagcttgaaatcatccctgcgagcctatcctgcccacgtgttgagatcattgccacgatgaaaaagaatgatgagcagagatgtctgaatccggaatctaagaccatcaagaatttaatgaaagcgtttagccaaaaaaggtctaaaagggctcctggtcagtcctctagatcttccggcggtggtggcagctccggtggtggcggttccttagcctatggaagtcagggtaatcttaatccattaattaatgaaatcagcaaaatcatttcagctgcaggtaattttgatgttaaagaggaaagagctgcagcttctttattgcagttgtccggtaatgccagtgatttttcatatggacggaactcaataactttgacagcatcagcataatttattaatttaaataatagcaatcttactgggctgtgccacataagattgctatttttttggagtcataatggattcttgtcataaaattgattatgggttatacgccctggagattttagcccaataccataacgtctctgttaacccg

SEQ ID NO: 7 - DNA, 557 bp, synthetic construct
  source 1..557: mol_type = other DNA
tcaaatggatccagatcgatgtttaaagatcccccctcactcctgccatcattctgatattgaaataagagaggaggaacataaatggctcaagttcaattagtagaaacagggggaggcttagttcagcctggaggatctcttcgcttatcttgcactgcttcaggcttcacattctcgatgcacgcgatgacctggtaccgtcaagcgccggggaagcagcgtgaattggtggcagttattacgagtcatggggaccgggcgaactacactgatagcgtccgtgggcgtttcacgattagccgcgacaataccaaaaacatggtatacttacagatgaatagcttaaaaccggaagatacggctgtgtattactgtaacgttccacggtacgattcatgggggcagggtactcaagtcactgtcagcagtggcggcttaccggaaacagggggtcaccaccaccatcatcacggtgcgtacccctatgatgtgcctgactatgcgagcttagcctatggaagtcagggtaatcttaatccattaattaacctctc

SEQ ID NO: 8 - DNA, 380 bp, synthetic construct
  source 1..380: mol_type = other DNA
ccagtccacagtgtgcggaataatgatgcataaagcggctatttcgccgcctaagaaaaagatcgggggaagtgaaaaattttctaaagttcgaaattcaggtgccgatacaagggttacggtgagaaaccgtgggcaacagcccaataacatcaagttgtaattgataaggaaaagatcatggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtccgctgggcctggcgggaaccacgtgtccacctcccgtatctattgagcatgcttccag

SEQ ID NO: 9 - DNA, 1053 bp, synthetic construct
  source 1..1053: mol_type = other DNA
gactgaggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcgtagtggctattgcagcgcttatcgggcctgcgtgtggttctgtaggccggataaggcgcgtcagcgccgccatccggcggggaaatttgtgttaaaccaggggtgcatcgtcaccctttttttgcgtaatacaggagtaaacgcagatgtttcatttttatcaggagttaagcagagcattggctattctttaagggtagcttaatcccacgggtattaagcctaacctgaaggtaggacgacgcagataggatgcacagtgtgcggaataatgatgcataaagcggctatttcgccgcctaagaaaaagatcgggggaagtgaaaaattttctaaagttcgaaattcaggtgccgatacaagggttacggtgagaaaccgtgggcaacagcccaataacatcaagttgtaattgataaggaaaagatcatggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtccgctgggcctggcgggactgactcagccgtcctcggtgtcagcaaacctgggaggaaccgtcaagatcacctgctccgggggtagtggcagctacggctggtatcagcagaaggcacctggcagtgcccctgtcagtctgatctatgacaacaccaacagaccctcggacatcccttcacgattctccggtgccctatccggctccacagccacattaaccatcactggggtccaagccgaggacgaggctgtctattactgtgggagcagggacagcagtaatgctggttctgtatttggggccgggacaaccctgaccgtcctaggtcagtc

SEQ ID NO: 10 - DNA, 1070 bp, synthetic construct
  source 1..1070: mol_type = other DNA
tggttgatgcataaagcggctatttcgccgcctaagaaaaagatcgggggaagtgaaaaattttctaaagttcgaaattcaggtgccgatacaagggttacggtgagaaaccgtgggcaacagcccaataacatcaagttgtaattgataaggaaaagatcatggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtccgctgggcctggcgggactgactcagccgtcctcggtgtcagcgaacccgggagaaaccgtcgagatcacctgctccgggggtagcaactactatggctggtaccagcagaagtcacctggcagtgcccctgtcactctgatctatagcagcaccaacagaccctcaaacatcccttcacgattctccggttccaaatccggctccacacacacattaaccatcactggggtccaagccgacgacgaggctgtctattactgtgggagtgtagacagcagctatgttggtgtatttggggccgggacaaccctgaccgtcctaggtcagtcctctagatcttccggcggtggtggcagctccggtggtggcggttccgccctgacgttggacgagtccgggggcggcctccagacaccgaaaggagggctcagcctcgtctgcaaggcctccgggttcaccttcagcagcttcaacatctactgggtgcgacaggcgcccggcaaggggctggaatacgtcgctgaaattagcggcactggcagttacacaaactacgggccggcggtggatggccgtgccaccatctcgagggacaacgggcagagcacagtgaggctgcagctgagcaatctcagggcagaggacaccgccacctactactgcgccaaagctgctggcggttgtagtggttatggttggtgtcctgctggcatcgacgcatggggccacgggaccgaagtcatcgtctcctccactagttgctag

SEQ ID NO: 11 - DNA, 991 bp, synthetic construct
  source 1..991: mol_type = other DNA
ccagtccacagtgtgcggaataatgatgcataaagcggctatttcgccgcctaagaaaaagatcgggggaagtgaaaaattttctaaagttcgaaattcaggtgccgatacaagggttacggtgagaaaccgtgggcaacagcccaataacatcaagttgtaattgataaggaaaagatcatggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtccgttaggacttgcaggcaaatccgccgttttatttttactggggattatctttctggagcaatgcggcgtacgcgggaccctggtcatccgcaacgcacgctgctcgtgcatctctacttcccggggaacaattcattataagtccttgaaggacttgaagcagtttgcgccgtcacctaactgcaataagactgagattattgcaacccttaaaaatggggaccaaacctgcttggaccctgacagcgcaaatgtcaagaaactgatgaaagaatgggaaaagaaaatttcccagaaaaagaaacaaaaacggggtaagaagcatcagaaaaatatgaaaaatcgtaaaccaaagaccccacagagccgtcggcgtagtcgtaaaacgacagtgccaggcgttggtgtaccgggagtcgggggcatcccattagcccggacggttcggtgcaactgcattcacatcgacgatggccctgtacgtatgcgggccattggaaaattggaaatcatcccagccagtctttcctgcccacgggttgaaatcattgcgactatgaagaagaatgatgagcaacgttgccttaacccggaatccaaaactatcaaaaacctgatgaaggctttcagtcaaaaacgttctaagcgggcaccttaagcttgacacg

SEQ ID NO: 12 - DNA, 727 bp, synthetic construct
  source 1..727: mol_type = other DNA
ccagtccacagtgtgcggaataatgatgcataaagcggctatttcgccgcctaagaaaaagatcgggggaagtgaaaaattttctaaagttcgaaattcaggtgccgatacaagggttacggtgagaaaccgtgggcaacagcccaataacatcaagttgtaattgataaggaaaagatcatggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtccgcttggtttggctggtcaggttcaacttgttgaaagcggaggcggattagctcaacccggcggctctttacgtctttcctgcgctgcctctgggtccactattagctccgttgccgtgggttggtaccgtcaaaccccaggcaaccaacgtgaatgggttgcaacatccagcaccagtagcacaaccgccacctatgcagactcggtaaaggggcgcttcacgattagtcgtgacaacgcaaaaaacaccatctaccttcaaatgaactctctgaagccagaggacacagccgtgtactattgcaagacgggacttacaaattgggggcgcggaacgcaagttaccgtaagctcgggtggtggctacccgtatgacgtgcctgactacgcctaagcttgacacg

SEQ ID NO: 13 - DNA, 730 bp, synthetic construct
  source 1..730: mol_type = other DNA
ccagtccacagtgtgcggaataatgatgcataaagcggctatttcgccgcctaagaaaaagatcgggggaagtgaaaaattttctaaagttcgaaattcaggtgccgatacaagggttacggtgagaaaccgtgggcaacagcccaataacatcaagttgtaattgataaggaaaagatcatggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtccattaggtttagccggccaagtccagcttgtcgaaaccggaggtggactggtgcagcctggagggtcactgcgcctttcctgtaccgcatctggtttcactttcagtatgcacgctatgacttggtatcgtcaggctcctggtaagcaacgtgaacttgtcgcggtcattacgagccatggagatcgcgcgaactatacggactcagtacgtggtcgctttacaatctcccgcgacaacactaagaatatggtctatttgcaaatgaatagcctgaagcctgaagatacagcggtctattattgtaacgtacctcgttacgacagttggggccaggggactcaagtaacggtttcatctggtggaggctatccctatgatgtgccagattacgcgtaagcttgacacg

SEQ ID NO: 14 - DNA, 751 bp, synthetic construct
  source 1..751: mol_type = other DNA
ccagtccacagtgtgcggaataatgatgcataaagcggctatttcgccgcctaagaaaaagatcgggggaagtgaaaaattttctaaagttcgaaattcaggtgccgatacaagggttacggtgagaaaccgtgggcaacagcccaataacatcaagttgtaattgataaggaaaagatcatggcgcaggtaatcaatacaaactccttaagtcttttaactcaaaataatcttaacaagagtcaatctgcgcttggcactgctatcgagcggttgtctagcgggttacgcatcaattctgccaaggatgatgcggcgggtccacttggcttagcgggccaagtgcagttggtggaatctggcggcggtttggtcgagccgggtggatcgttacggttatcttgtgctgcatcgggaatcatcttcaagatcaacgacatggggtggtaccggcaggcaccaggtaaacgtcgtgaatgggtagcagcgtcgactggtggggatgaagccatttatcgcgattccgttaaggaccgttttactattagtcgcgacgccaagaattccgtttttttacagatgaatagtttgaaaccggaggataccgccgtctattactgtactgctgtaatctcaacagaccgggatggtacagagtggcgtcgctattggggccaggggacgcaggttacagtgagttctggtggtggctatccgtacgatgtcccggattacgcgtaagcttgacacg

SEQ ID NO: 15 - DNA, 1555 bp, synthetic construct
  source 1..1555: mol_type = other DNA
ccagtccacagtgtgcggaataatgatgcataaagcggctatttcgccgcctaagaaaaagatcgggggaagtgaaaaattttctaaagttcgaaattcaggtgccgatacaagggttacggtgagaaaccgtgggcaacagcccaataacatcaagttgtaattgataaggaaaagatcatggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtccgttaggactggctggtacgacctgtcctcccccagtttcaatcgagcacgcggatatccgcgttaaaaactactccgtcaatagtcgtgaacgttatgtatgcaattccggtttcaagcgtaaagctgggacatctaccttgattgagtgcgtaatcaacaaaaacaccaacgtggcccactggacgacgccgtccttgaagtgcattcgggatccctctttagcgcattacagtccggtgccaacgtcaggtggtagtggcggaggcggttcaggcggcgggtctggaggtggtgggagcttacagaactggatcgatgtgcggtatgatttagagaagattgaatccttgatccaatctattcacattgacacaaccttgtatactgattcagattttcacccctcttgcaaggttacggctatgaattgttttctgctggagttacaagtgattcttcacgagtactctaatatgactttgaatgaaacagtccgcaatgtgttgtatcttgctaactccaccctgagcagtaacaagaacgttgctgaaagcggttgcaaagaatgtgaggagcttgaggagaagaccttcacggagttcctgcagtcattcattcgcattgttcaaatgtttattaacacttcctgattgttggaatacgccataaactgggcgggtatggcgcaggtcattaacactaatagtctgagtctgttgacacagaacaacttaaataagtctcagagcgctttaggcaccgctattgaacgtctgagttcggggcttcgcatcaattcagcaaaagatgacgcagcgggacctttagggttggcaggtcaagtccagcttgttgaaaccggcggcggcttagttcagcccggcggttccctgcgtttaagttgcactgcctcgggatttaccttcagcatgcacgcaatgacttggtatcggcaagctccaggaaagcagcgggaattagtcgcagtaattactagccatggagaccgggcaaattacacagactccgtgcgcggacggttcacaatttcccgcgacaatactaagaacatggtttatttacaaatgaattcattaaagcctgaggacaccgccgtctactactgcaatgtcccacggtatgacagttgggggcaaggcacccaagtaacggtctcctcgggtggcggttatccttacgatgtccccgattatgcgtaagcttgacacc

SEQ ID NO: 16 - DNA, 1960 bp, synthetic construct
  source 1..1960: mol_type = other DNA
ccagtccacagtgtgcggaataatgatgcataaagcggctatttcgccgcctaagaaaaagatcgggggaagtgaaaaattttctaaagttcgaaattcaggtgccgatacaagggttacggtgagaaaccgtgggcaacagcccaataacatcaagttgtaattgataaggaaaagatcatggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtccgctcgggctggcggggatgtgggaactcgaaaaggatgtgtacgttgttgaagttgactggactcccgacgctccaggggaaacagtaaacttaacttgtgatactcccgaagaggacgatatcacgtggacttccgaccaacggcacggcgtaatcggctccgggaagacccttactattacagttaaggaattcctggacgctggtcagtatacttgtcacaaaggcggtgaaactctgtcccatagccatttactgctgcacaaaaaggaaaacggcatttggagcactgagatccttaaaaatttcaagaataaaacctttctgaaatgcgaagcgcctaactatagcggtcggtttacttgttcctggttggtacagcgtaacatggacttgaaattcaatatcaagtcgtcttcctcgagccctgattcgcgtgcagtaacgtgcggcatggcgtcgttatccgccgagaaagtcacattagatcagcgggactatgagaagtatagcgttagttgtcaagaggacgtgacgtgccccacggctgaggaaacgttaccgattgaattagcgcttgaagcccgccaacagaataaatacgaaaactatagcacgtctttctttatccgcgatatcattaaacctgatcccccgaaaaatttacagatgaaaccgttgaaaaactcacaggttgaggtatcctgggaatatccggactcttggagtacaccgcattcgtacttttcgcttaagttctttgtacgtattcaacggaaaaaggagaagatgaaggagaccgaggagggatgcaatcaaaagggagctttcttagtcgagaagacatctacggaagtgcagtgcaaggggggcaacgtctgtgtacaggcacaggatcgctactacaattcgtcttgcagcaagtgggcgtgtgttccctgccgtgtacggagtggcggaggtggctctggcggggggggcagcggcgggggcggctcaaaccacctttccttagcacgggtgatcccggtaagcggcccagcgcgctgtctgtcccaaagtcggaatctgttaaagaccacagacgacatggtcaagaccgcccgtgagaagttaaagcactatagttgtacagctgaagacattgaccacgaagacatcactcgcgaccaaacatcaacacttaaaacctgcttaccgttagaacttcataaaaatgagagctgcttggcgacccgtgaaacgtcaagcactactcggggaagttgcttgcccccacagaagacttcgttaatgatgaccctgtgcttaggtagtatctatgaggaccttaagatgtaccaaactgagttccaagcaatcaacgcagcgttacaaaatcataaccatcaacaaatcattcttgataaggggatgctggtggctattgacgaacttatgcagtcacttaatcataatggcgaaaccctccgacaaaagccgccggtgggagaagcggatccttaccgtgtgaagatgaaactgtgcattcttcttcatgccttttccacgcgtgtggtgacgatcaatcgggtgatgggatatttgtccagtgcgggtggtggccatcatcaccatcaccattaagcttgacacg

SEQ ID NO: 17 - DNA, 2663 bp, synthetic construct
  source 1..2663: mol_type = other DNA
gataagacgcgtgtggtgacgatcaatcgggtgatgggatatttgtccagtgcgtaatttgccagtctgaggcggtagatctatggctcaagtaattaacacaaactcgctttccctgttgacgcagaataacctgaataaatcacagtccgcattgggtaccgccattgagcggttaagctctggtttacggatcaactctgcgaaagatgatgctgcgggcccgctcggattggcgggccactttggtcgtttgcactgtacgactgccgttatacggaacatcaatgaccaagtcctatttgtggacaagcgccagccagtttttgaagacatgaccgacatcgatcagtcagcgtccgaacctcagacgcggcttattatctatgcgtatggtgactctcgagcccgcggaaaagctgtgacgctgagtgtaaaggacagcaagatgtcaacattgtcatgtaagaacaagatcatctctttcgaagaaatggacccccctgaaaacattgatgacatccagagcgatcttatcttcttccagaaacgggtgcccggtcataataagatggaatttgagtcgagcctgtatgagggccatttccttgcttgccaaaaggaggacgatgccttcaaattgatcctgaagaaaaaggatgaaaacggagataaatcagtaatgtttacccttaccaacttacaccagtcttaacccgtaaggtaaggaactaacaaatatggcacaggtcattaacacgaatagtctgtctctgttaacgcaaaataacttgaataagtcccagtcggctttggggaccgcaatcgagcgcttgtcaagtggtcttcgtattaactctgcaaaagacgatgcagcaggtccgttaggactggctggtacgacctgtccccccccagtttcaatcgagcacgcggatatccgcgttaaaaactactccgtcaatagtcgtgaacgttatgtatgcaattccggtttcaagcgtaaagctgggacatctaccttgattgagtgcgttatcaacaaaaacaccaacgtggcccactggacgacgccgtccttgaagtgcattcgggatccctctttagcgcattacagtccggtgccaacgtcagggggcagtggaggcggcggttcgggtggaggctctggaggtggtgggagcttacagaactggatagatgtgcggtatgatttagagaagattgaatccttgatccaatctattcacattgacacaaccttgtatactgattcagattttcacccctcttgcaaggttacggctatgaattgttttctgctggagttacaagtgattcttcacgagtactctaacatgactctgaatgaaacggttcgcaacgtgttgtatcttgctaactccaccctgagcagtaacaagaacgttgctgagagcggttgcaaagaatgtgaggagcttgaggagaagaccttcacggagttcctgcagtcattcattcgcatcgttcaaatgtttattaatacttcatgattgtaacgtttaagaaagcctcccaatggcgcaagtgattaacactaatagtctgagtctgttgacacagaacaacttaaataagtctcagagcgctttaggcaccgctattgaacgtctgagttcggggcttcgcatcaattcagcaaaagatgacgcagcgggacctttagggttggcaggtcaggtccagcttgtagaaacaggcggcggcttagttcagcctggcggttccctgcgtttaagttgcactgcctcgggatttaccttcagcatgcacgcaatgacttggtatcggcaagctccaggaaagcagcgggaattagtcgcagtaattacttcgcatggagaccgggcgaattacacagactccgtgcgcggacgcttcacaatttcccgcgacaataccaaaaacatggtttatctgcaaatgaattccctgaaaccagaggataccgctgtttactactgcaatgtcccacggtatgacagttgggggcagggcacccaagtaacggtctcctcgggtggcggttatccttacgatgtccccgattatgcgtaaatcgtaagattaagccgagactctcatggcgcaggtaatcaacaccaattcccttagccttctgacgcaaaacaatcttaacaaatctcagtctgccttgggcacggctattgagcgcctgtcatctggcttaagaatcaattctgcgaaggatgatgcggcgggtccgcttggtttggctggtcaggttcaacttgttgaaagcggaggcggattagctcaacccggcggctctttacgtctttcctgcgctgcctctgggtccactattagctccgttgccgtgggttggtaccgtcaaaccccaggcaaccaacgtgaatgggttgcgacatccagcaccagtagcacaaccgccacgtatgcagactcggtaaaggggcgcttcacgattagtcgtgacaacgcaaagaacacgatctaccttcagatgaactctctgaagccagaggacacagccgtgtactattgtaagacgggacttacaaattgggggcgcggaacgcaggttaccgtaagctcgggtggtggccatcatcaccatcaccattaagcttgacacc

SEQ ID NO: 18 - DNA, 29 bp, synthetic construct
  source 1..29: mol_type = other DNA
actgttaattaagaagaccttccattctg

SEQ ID NO: 19 - DNA, 40 bp, synthetic construct
  source 1..40: mol_type = other DNA
aggattctcgagttatcaactatcaaacgcttcggttaag

SEQ ID NO: 20 - DNA, 75 bp, synthetic construct
  source 1..75: mol_type = other DNA
aagtgtaatgttctaacaggtctcttctaggagctggcgttctcccttctgatgagtgtaggctggagctgcttc

SEQ ID NO: 21 - DNA, 75 bp, synthetic construct
  source 1..75: mol_type = other DNA
ccataagcacaatccgctgctcaagtcatcagaagggagaacgccagctcctagagtgtaggctggagctgcttc

SEQ ID NO: 22 - DNA, 75 bp, synthetic construct
  source 1..75: mol_type = other DNA
ttgctagttccgtcaagggatcacgggtaggagccaccttgtacccttgtcaaaagtgtaggctggagctgcttc

SEQ ID NO: 23 - DNA, 75 bp, synthetic construct
  source 1..75: mol_type = other DNA
aaggccagtcgacagactggcctttttttgacaagggtacaaggtggctcctaccgtgtaggctggagctgcttc

SEQ ID NO: 24 - DNA, 29 bp, synthetic construct
  source 1..29: mol_type = other DNA
cggggtaccagtatgacgttctgacggtt

SEQ ID NO: 25 - DNA, 29 bp, synthetic construct
  source 1..29: mol_type = other DNA
cggggtaccattttcagctctgaagtaca

SEQ ID NO: 26 - DNA, 75 bp, synthetic construct
  source 1..75: mol_type = other DNA
attcggggctttttcatttagcatagatgaatatatatttcgcctacggtaataagtgtaggctggagctgcttc

SEQ ID NO: 27 - DNA, 75 bp, synthetic construct
  source 1..75: mol_type = other DNA
agttttacttttctcacggaattttttattaccgtaggcgaaatatatattcatcgtgtaggctggagctgcttc

SEQ ID NO: 28 - DNA, 75 bp, synthetic construct
  source 1..75: mol_type = other DNA
actggatggcgaatagcgccctaaccatgggactggcgtacctttctgtttgccggtgtaggctggagctgcttc

SEQ ID NO: 29 - DNA, 75 bp, synthetic construct
  source 1..75: mol_type = other DNA
agacggttaatcaccggttaaacaccggcaaacagaaaggtacgccagtcccatggtgtaggctggagctgcttc

SEQ ID NO: 30 - DNA, 75 bp, synthetic construct
  source 1..75: mol_type = other DNA
gccgataacaacgagtattgaaggattaaaaggaaccatccacctctttttgaaagtgtaggctggagctgcttc

SEQ ID NO: 31 - DNA, 75 bp, synthetic construct
  source 1..75: mol_type = other DNA
aaaacatatccagtttcgtgatatgtttcaaaaagaggtggatggttccttttaagtgtaggctggagctgcttc

SEQ ID NO: 32 - DNA, 75 bp, synthetic construct
  source 1..75: mol_type = other DNA
ctgcgcatcctgtcatagtcaacagcggaaggatgatgtctagcgtgagcatggagtgtaggctggagctgcttc

SEQ ID NO: 33 - DNA, 75 bp, synthetic construct
  source 1..75: mol_type = other DNA
aaatgtctgataaaaatcgctaatatccatgctcacgctagacatcatccttccggtgtaggctggagctgcttc

SEQ ID NO: 34 - DNA, 167 bp, synthetic construct
  source 1..167: mol_type = other DNA
ggatgcttcatttaaatgggtgaacaaggaaagctaaaagggatcaggtaaatttgatgtacatcaaatggatccagatcgatgtttaaagatcccccctcactcctgccatcattctgatattgaaataagagaggaggaacataagtgtaggctggagctgcttc

SEQ ID NO: 35 - DNA, 167 bp, synthetic construct
  source 1..167: mol_type = other DNA
aaatgtgttttagcaactcggatgtatgcattgttcccatttatgttcctcctctcttatttcaatatcagaatgatggcaggagtgaggggggatctttaaacatcgatctggatccatttgatgtacatcaaatttacctgatccgtgtaggctggagctgcttc

SEQ ID NO: 36 - DNA, 75 bp, synthetic construct; identical to SEQ ID NO: 20
  source 1..75: mol_type = other DNA

SEQ ID NO: 37 - DNA, 75 bp, synthetic construct; identical to SEQ ID NO: 21
  source 1..75: mol_type = other DNA

SEQ ID NO: 38 - DNA, 110 bp, synthetic construct
  source 1..110: mol_type = other DNA
actgttaattaattatctctggcggtgttgacataaataccactggcggtgatactgagcacatcagcaggacgcactgacccacaataggaggaacataaatgactaaa

SEQ ID NO: 39 - DNA, 40 bp, synthetic construct; identical to SEQ ID NO: 19
  source 1..40: mol_type = other DNA

SEQ ID NO: 40 - DNA, 29 bp, synthetic construct
  source 1..29: mol_type = other DNA
cggggtaccagtatgacgttctgacggtt

SEQ ID NO: 41 - DNA, 29 bp, synthetic construct
  source 1..29: mol_type = other DNA
cggggtaccattttcagctctgaagtaca

SEQ ID NO: 42 - DNA, 75 bp, synthetic construct
  source 1..75: mol_type = other DNA
gacaacgcatggcgtaatgccataatcaggaaataaaactgtacaaatgtagtacgtgtaggctggagctgcttc

SEQ ID NO: 43 - DNA, 75 bp, synthetic construct
  source 1..75: mol_type = other DNA
ccgtctgtcgacggccttctgcctggtactacatttgtacagttttatttcctgagtgtaggctggagctgcttc

SEQ ID NO: 44 - DNA, 1059 bp, synthetic construct
  source 1..1059: mol_type = other DNA
gcaacgtcatgagacaataaccctgataaatgcttcaataatattggaaaaggaagagtatgtgtggaattgttggcgctatcgcgcagcgtgatgtagctgaaatccttctcgaaggtttacgtcgtctggaataccgcggatatgactctgccggtctggccgtggtggatgcagaaggtcatatgacccgcctgcgtcgcctcggtaaagtccagatgctggctcaggcagcggaagaacatcctctgcatggcggcactggtattgctcacactcgctgggcgacgcacggtgaaccttcagaagctaacgcgcatccgcatgtttctgaacacattgtggtggtgcataacggcatcatcgaaaaccatgaaccgctgcgtgaagcgttaaaagcgcgtggctataccttcgtttctgaaaccgacaccgaagtgattgcacacctggtaaactgggaactgaaacagggcggtactctgcgtgaggctgttctgcgtgctatcccgcaactgcgtggcgcatatggtacggtaattatggatacccgtcacccggataccctactggcggcacgttgctggtagcccgctggtgattggcctgggtatgggcgaaaactttatcgcttctgaccagctggcgctgttgccagtgactcgtcgctttatcttccttgaagagggcgatatcgctgaaatcactcgccgttcggtaaatatcttcgataacaccggcgcggaagtaaaacgtcaggatatcgaatccaatctgcaatatgacgcgggcgataaaggtatttaccgccactacatgcagaaagagatctacgaacagccgaacgcgatcaaaaacacccttaccgggcgcatcagccacggtcaggtggatttaagcgagctgggaccgaacgccgacgaactgctgtcgaaggttgagcatattcagatcctcgcctgcggtacgtcttacaactccggtatggtttcccgttactggtttgaatcgctggcaggtattccgtgcgacgttgaaatcgcctctgaattcggtagt

SEQ ID NO: 45 - DNA, 1197 bp, synthetic construct
  source 1..1197: mol_type = other DNA
cctaccgaattccgttatcgcaaatctgccgtgcgccgtaacagcctgatgatcaccctgtcacagtctggcgaaaccgcagataccctggctggcctgcgtctgtcgaaagagctgggatatttgggctctctggcaatttgtaacgtgccgtcttcgctggtgcgtgaatccgatctggcgctgatgaccaacgcgggtacggaaattggcgtagcatctaccaaagccttcaccacccagttaaccgtgctgttgatgctggtggcgaaactgtctcgcctgaaaggtctggatgcgtccattgagcatgatatcgttcatggcctacaggcgctgccgagccgtattgagcagatgctgtcgcaggacaaacgcatcgagctgcttgcagaagatttctccgacaagcatcacgcgctattcctggggcgtggcgatcaatatccgattgcgctcgaaggcgcgctgaagctgaaagagatctcttacattcacgcggaagcctatgctgcaggcgagctgaaacacgggccgttggcgctgattgacgccgatatgccggtgattgtggtggcgccgaacaacgaactgctggagaaactgaaatccaacacgaagaagttcgcgcacgtggcggtcagttgtacgtcttcgccgatcaggatgcgggtttcgtcagcaacgacaacatgcacatcatcgagatgccgcatgtggaagaggtcattgcgcctatcttctacaccgttccgctgcaactgctggcgtatcacctggcgctgattaaaggcaccgacgttgaccagccgcgtaacctggcgaaatcggttaccgtagagtaactgtcagaccaagtttactcatatatactttagattgatttaaaacttcatttttaatttaaaaggatctaggtgaagatcctttttgataatctgatgaccaaaatcccttaacgtgagttttcgttccactgagcgtcagaccccgtragaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttc

SEQ ID NO: 46 - DNA, 1129 bp, synthetic construct
  source 1..1129: mol_type = other DNA
aaggtggctagcgtgcggaataatgatgcataaagcggctatttcgccgcctaagaaaaagatcgggggaagtgaaaaattttctaaagttcgaaattcaggtgccgatacaagggttacggtgagaaaccgtgggcaacagcccaataacatcaagttgtaattgataaggaaaagatcatggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtcccttaggtttagcggggtgtgacttaccgcacacgtacaacttgcggaacaagcgggcgttgaaagtgctggctcagatgcggctgcttaccattcttatcctgcttgaaagatcggcaagacttcgggttcccattagagaaggtggataaccaacaaatccagaaagcgcaagctatcccagttcttcgagacttgacccagcaaacgctgaatttgtttacttctaaagcctcctccgcggcatggaatacgactttgttggattctttctgcaacgacctccaccaacagttgaacgatttacagacgtgcctgatgcagcaagttggagtccaagaaccacccttaacacaggaagacgcgctgttagctgtccgtaaatactttcaccgtgccctttcttcttccgtgaacttgttgcctcggttgtcagaagagaaagaataagaaacgcgcctaaggaggtcctcacatggcgcaggttatcaacacgaactccttaagcctgttgactcagaacaacttgaacaaaagccaatcagcactcggaacagcgatagaacgcttatcgtcgggattgcggatcaattctgccaaagacgatgcggccggacccttgggtctggctggggatcccgtgccacgtgccacacgtctgccggttgaagccaaagactgccatattgctcagtttaagtccttatcacctaaggaattgcaagcgtttaagaaggctaaggacgcgatagagaaacgattgttagaaaaggatcttcggtgtagctctcatttgtttcctcgtgc

SEQ ID NO: 47 - DNA, 33 bp, synthetic construct
  source 1..33: mol_type = other DNA
atgactaaaaaaatttcattcattattaacggc

SEQ ID NO: 48 - DNA, 28 bp, synthetic construct
  source 1..28: mol_type = other DNA
ttatcaactatcaaacgcttcggttaag

SEQ ID NO: 49 - DNA, 21 bp, synthetic construct
  source 1..21: mol_type = other DNA
ttatgggctgttttggcggat

SEQ ID NO: 50 - DNA, 31 bp, synthetic construct
  source 1..31: mol_type = other DNA
ttatgttcctcctattgtgaaattgttatcc

SEQ ID NO: 51 - DNA, 55 bp, synthetic construct
  source 1..55: mol_type = other DNA
caacctgtatccgtccggcgtcataaaaggaaaaatagagggaggcataatgatc

SEQ ID NO: 52 - DNA, 55 bp, synthetic construct
  source 1..55: mol_type = other DNA
cgatagcgatgaaaacgcgccgaaggatcattatgcctccctctatttttccttt

SEQ ID NO: 53 - DNA, 55 bp, synthetic construct
  source 1..55: mol_type = other DNA
cgccgcctaagaaaaagatcgggggaagtgaaaaattttctccggcgattgattc

SEQ ID NO: 54 - DNA, 55 bp, synthetic construct
  source 1..55: mol_type = other DNA
ccttgattgtgtaccacgtgtcggtgaatcaatcgccggagaaaatttttcactt

SEQ ID NO: 55 - DNA, 55 bp, synthetic construct
  source 1..55: mol_type = other DNA
gaggattgctttatcaaaaaccttccaaaaggaaaatttttttatttcgttttat

SEQ ID NO: 56 - DNA, 55 bp, synthetic construct
  source 1..55: mol_type = other DNA
aaaagccccgaattcacggggctgaataaaacgaaataaaaaaattttccttttg

SEQ ID NO: 57 - DNA, 55 bp, synthetic construct
  source 1..55: mol_type = other DNA
aagagactctgtctcatcccaaacctattgtggagaaaagaaaaacgcgctgata

SEQ ID NO: 58 - DNA, 55 bp, synthetic construct
  source 1..55: mol_type = other DNA
gaaaacgcgctgataccgtaataagtatcagcgcgtttttcttttctccacaata

SEQ ID NO: 59 - DNA, 40 bp, synthetic construct
  source 1..40: mol_type = other DNA
attcagatgtaatctggagaggtgatgaagttttcatctt

SEQ ID NO: 60 - DNA, 66 bp, synthetic construct
  source 1..66: mol_type = other DNA
ttctctggcgtaataaatgtactcttgtccgacgatttgataagatgaaaacttcatcacctctcc

SEQ ID NO: 61 - DNA, 59 bp, synthetic construct
  source 1..59: mol_type = other DNA
cggcctacagaaccacacgcaggcccgataagcgctgcaaattgtgtaggctggagctg

SEQ ID NO: 62 - DNA, 66 bp, synthetic construct
  source 1..66: mol_type = other DNA
cacaggatactggcgcgcatacacagcacatctctttgcagtccatatgaatatcctccttagttc

SEQ ID NO: 63 - DNA, 68 bp, synthetic construct
  source 1..68: mol_type = other DNA
ggacgacgcagataggatgcacagtgtgctgcgccgttcaggtcaaagaagtgtcactacctgatgtt

SEQ ID NO: 64 - DNA, 69 bp, synthetic construct
  source 1..69: mol_type = other DNA
gaaggatcaggtaaatttgatgtacatcaaatggatccagatcgatgtttaaagatcccccctcactcc

SEQ ID NO: 65 - DNA, 91 bp, synthetic construct
  source 1..91: mol_type = other DNA
tgccatcattctgatattgaaataagagaggaggaacataaatgactaaaaaaatttcattcattattaacggccaggttgaaatctttcc

SEQ ID NO: 66 - DNA, 35 bp, synthetic construct
  source 1..35: mol_type = other DNA
cgaaagtgatgatttagtgcaatccattaattttg

SEQ ID NO: 67 - DNA, 66 bp, synthetic construct
  source 1..66: mol_type = other DNA
gtgataatagtgtttacctgccaatattgaatgactctcatgtaaaaaacattattgattgtaatg

SEQ ID NO: 68 - DNA, 39 bp, synthetic construct
  source 1..39: mol_type = other DNA
gaaataacgaattacggttgcataacattgtcaattttc

SEQ ID NO: 69 - DNA, 68 bp, synthetic construct
  source 1..68: mol_type = other DNA
tctatacggtagggcaaagatggaaaaatgaagaatactcaagacgcaggacatacattcgtgactta

SEQ ID NO: 70 - DNA, 57 bp, synthetic construct
  source 1..57: mol_type = other DNA
aaaaaatatatgggatattcagaagaaatggctaagctagaggccaattggatatct

SEQ ID NO: 71 - DNA, 68 bp, synthetic construct; identical to SEQ ID NO: 63
  source 1..68: mol_type = other DNA

SEQ ID NO: 72 - DNA, 70 bp, synthetic construct
  source 1..70: mol_type = other DNA
gaattggataacgcctctttgtcagaacctgtccattcgttgacgcacatcaagatagctttcattcgaa

SEQ ID NO: 73 - DNA, 70 bp, synthetic construct
  source 1..70: mol_type = other DNA
agtaatttaatctttatatgaaataagagaggaggaacataaatgactaaaaaaatttcattcattatta

SEQ ID NO: 74 - DNA, 69 bp, synthetic construct
  source 1..69: mol_type = other DNA
acggccaggttgaaatctttcccgaaagtgatgatttagtgcaatccattaattttggtgataatagtg

SEQ ID NO: 75 - DNA, 70 bp, synthetic construct
  source 1..70: mol_type = other DNA
tttacctgccaatattgaatgactctcatgtaaaaaacattattgattgtaatggaaataacgaattacg

SEQ ID NO: 76 - DNA, 69 bp, synthetic construct
  source 1..69: mol_type = other DNA
gttgcataacattgtcaattttctctatacggtagggcaaagatggaaaaatgaagaatactcaagacg

SEQ ID NO: 77 - DNA, 78 bp, synthetic construct
  source 1..78: mol_type = other DNA
caggacatacattcgtgacttaaaaaatatatgggatattcagaagaaatggctaagctagaggccaattggatatct